(19) United States
(12) Patent Application Publication
(10) Pub. No.: US 2009/0226956 A1
(43) Pub. Date: Sep. 10, 2009

(54) RUNX-MEDIATED ACTION OF NUCLEAR RECEPTORS AND UTILITIES THEREOF

(75) Inventors: Baruch Frenkel, Glendale, CA (US); Gerhard A. Coetzee, La Canada, CA (US); Omar Khalid, Anaheim, CA (US); Sanjeev K. Baniwal, Alhambra, CA (US)

Correspondence Address: HOGAN & HARTSON L.L.P., 1999 AVENUE OF THE STARS, SUITE 1400, LOS ANGELES, CA, 90067, US

(73) Assignee: University of Southern California, Los Angeles, CA (US)

(21) Appl. No.: 12/367,493

(22) Filed: Feb. 6, 2009

Related U.S. Application Data
(60) Provisional application No. 61/026,927, filed on Feb. 7, 2008.
(60) Provisional application No. 61/042,685, filed on Apr. 4, 2008.

(51) Int. Cl. C12Q 1/02; C12N 5/06

(52) U.S. Cl. 435/29; 435/375

(57) ABSTRACT

The present invention discloses methods of identifying candidate compounds for modulating the activity of Runx2 or Runx1 through inhibition by ERα or AR in a cell, and using such compounds for treating bone diseases and cancer. Also disclosed are isolated polypeptides having fragments of a Runx2 protein or an AR protein involved in Runx2-ERα, Runx2-AR, or Runx1-AR interactions, their coding nucleic acids, and utilities of these polypeptides in modulating the activity of Runx2 or Runx1 through inhibition by ERα or AR in a cell.

### RELATED APPLICATIONS

This application claims priority to U.S. Provisional Application Ser. Nos. 61/026,927 and 61/042,685, filed on Feb. 7, 2008 and Apr. 4, 2008, respectively, the contents of which are incorporated herein by reference in their entirety.

### FUNDING

This invention was made with support in part by grants from the National Institutes of Health (DK071122, AR047052, and CA109147), the Department of Defense (W81XWH-05-1-0025, W81XWH-04-1-0823, and W81XWH-07-1-0067), the NIH/NCl (P30 CA 014089-30), and the NIH/NCRR (Research Facilities Improvement Program Grant Number C06 RR10600-01, CA62528-01, and RR14514-01). Therefore, the U.S. government has certain rights.

### FIELD OF THE INVENTION

The present invention relates in general to Runx-mediated action of nuclear receptors. More specifically, the invention provides drug screening, treatment, and diagnostic methods involving detection or modulation of the interaction between Runx1 (Runt-related transcription factor 1) or Runx2 (Runt-related transcription factor 2) and ERα (estrogen receptor a) or AR (androgen receptor).

### BACKGROUND OF THE INVENTION

Osteoporosis is one of the most spread degenerative diseases in Western Society. In 2004, the first-ever report on osteoporosis by the Surgeon General was released, stating that 10 million Americans over the age of 50 had osteoporosis and 34 million were at risk. The report predicted that by 2020, one of two Americans over the age of 50 would be at risk for fracture from osteoporosis or low bone mass. These statistics are alarming because of the high mortality and large economic burden associated with osteoporotic fractures.

It is well established that postmenopausal women are susceptible to bone fractures. We do not have a good understanding as to why bone mass decreases when women stop producing estrogens, the female sex hormones. We do know that the protective effect of estrogens on the skeleton is mediated by an estrogen receptor called ERα. We also know that activated ERα decreases the rate of bone turnover, a process occurring throughout life, in which osteoclasts degrade bone and osteoblasts compensate by depositing new bone material. Because bone turnover during adult life is imbalanced, with bone degradation exceeding bone formation, it is important to keep this process in check, a function carried out by estrogens. However, how they do it at the molecular level is unknown.

In men, bone turnover is kept in check by androgens, the male sex steroids. Androgens can be converted to estrogens, which then activate ERα, just like in females. In addition, androgens can activate the androgen receptor (AR), resulting in an extra protection to the skeleton not endowed by estrogens. As with ERα, the molecular mechanisms by which the AR slows bone turnover are not known.

### SUMMARY OF THE INVENTION

The present invention is based, at least in part, upon the unexpected discovery that the activity of a Runx protein can be inhibited by a steroid hormone receptor (SHR).

Accordingly, in one aspect, the invention features a method of identifying a candidate compound for reducing the activity of a Runx2 protein in a cell. The method comprises providing a system containing a Runx2 protein and an ERα protein, contacting the system with a test compound, and determining the inhibition of the activity of the Runx2 protein by the ERα protein in the system. If the inhibition of the activity of the Runx2 protein by the ERα protein in the system is increased compared to that before the contacting step, it indicates that the test compound is a candidate for reducing the activity of the Runx2 protein in a cell.

In some embodiments, a system of the invention is a cell. The cell may be osteoblastic.

In some embodiments, a compound of the invention activates the ERα protein; in other embodiments, a compound of the invention does not activate the ERα protein. The ERα protein may be transcriptionally incompetent. In some embodiments, the ERα protein contains a DBD (DNA-binding domain). In some embodiments, the Runx2 protein contains a PST (proline-serine-threonine) domain. A test compound of the invention may be a SERM (selective estrogen receptor modulator).

In another aspect, the invention features a method of reducing the activity of a Runx2 protein in a cell. The method comprises providing a system containing a Runx2 protein and an ERα protein, and contacting the system with a compound identified according to the method described above.

More specifically, a method of reducing the activity of a Runx2 protein in a cell comprises contacting an osteoblast with E2 (estradiol), OHT (hydroxytamoxifen), ICI 182780, or raloxifene, thereby reducing the activity of the Runx2 protein in the osteoblast. The inhibition of the activity of the Runx2 protein by an ERα protein in the osteoblast is increased by E2, OHT, ICI 182780, or raloxifene.

The invention also features a method of identifying a candidate compound for modulating the activity of a Runx2 protein in a cancer cell. The method comprises providing a cancer cell containing a Runx2 protein and an ERα protein, contacting the cell with a test compound, and determining the inhibition of the activity of the Runx2 protein by the ERα protein in the cell. If the inhibition of the activity of the Runx2 protein by the ERα protein in the cell is different from that before the contacting step, it indicates that the test compound is a candidate for modulating the activity of the Runx2 protein in the cancer cell.

A cancer of the invention may be breast cancer, prostate cancer, or leukemia. A cancer cell may be an early stage cancer cell or a late stage cancer cell.

The invention further features a method of modulating the activity of a Runx2 protein in a cancer cell. The method comprises providing a cancer cell containing a Runx2 protein and an ERα protein, and contacting the cell with a compound identified according to the method described above.

More specifically, a method of modulating the activity of a Runx2 protein in a cancer cell comprises contacting a cancer cell with E2, OHT, ICI 182780, or raloxifene, thereby modulating the activity of the Runx2 protein in the cell. The inhibition of the activity of the Runx2 protein by an ERα protein in the cell is modulated by E2, OHT, ICI 182780, or raloxifene.

In addition, the invention provides a method of detecting the interaction between a Runx2 protein and an ERα protein. The method comprises providing a system containing an ERα protein and a Runx2 protein, the Runx2 protein including a PST domain; and detecting the interaction between the ERα protein and the PST domain of the Runx2 protein.

Moreover, the invention provides an isolated polypeptide comprising an amino acid sequence at least 95% identical to amino acids 88-507, 88-425, 319-507, or 328-425 of a human Runx2 protein. The polypeptide has 98-506 amino acids in length. In some embodiments, the polypeptide consists of an amino acid sequence at least 95% identical to amino acids 88-507, 88-425, 319-507, or 328-425 of the human Runx2 protein.

The invention also provides an isolated polypeptide comprising an amino acid sequence at least 95% identical to amino acids 177-596, 177-514, 408-596, or 417-514 of a mouse Runx2 protein. The polypeptide has 98-595 amino acids in length. In some embodiments, the polypeptide consists of an amino acid sequence at least 95% identical to amino acids 177-596, 177-514, 408-596, or 417-514 of the mouse Runx2 protein.

The invention further provides an isolated nucleic acid comprising a nucleotide sequence encoding a polypeptide of the invention.

Also within the invention is a method of modulating the interaction between a Runx2 protein and an ERα protein. The method comprises providing a system containing a Runx2 protein and an ERα protein, and contacting the system with a polypeptide of the invention, thereby modulating the interaction between the Runx2 protein and the ERα protein. The inhibition of the activity of the Runx2 protein by the ERα protein is modulated by the polypeptide.

Furthermore, the invention provides a method of determining whether a subject is suffering from or at risk for developing a bone disorder or cancer. The method comprises detecting the inhibition of the activity of the Runx2 protein by the ERα protein in a subject. If the inhibition of the activity of the Runx2 protein by the ERα protein in the subject is decreased compared to a control level, it indicates that the subject is likely to be suffering from or at risk for developing a bone disorder or cancer.

In another aspect, the invention features a method of identifying a candidate compound for reducing the activity of a Runx2 protein in a cell. The method comprises providing a system containing a Runx2 protein and an AR protein, contacting the system with a test compound, and determining the inhibition of the activity of the Runx2 protein by the AR protein in the system. If the inhibition of the activity of the Runx2 protein by the AR protein in the system is increased compared to that before the contacting step, it indicates that the test compound is a candidate for reducing the activity of the Runx2 protein in a cell.

In some embodiments, a compound of the invention activates the AR protein; in other embodiments, a compound of the invention does not activate the AR protein. The AR protein may be transcriptionally incompetent. In some embodiments, the AR protein contains a DBD. In some embodiments, the Runx2 protein contains a Runt domain and a PST domain. A test compound of the invention may be a SARM (selective androgen receptor modulator).

In still another aspect, the invention features a method of reducing the activity of a Runx2 protein in a cell. The method comprises providing a system containing a Runx2 protein and an AR protein, and contacting the system with a compound identified according to the method described above.

More specifically, a method of reducing the activity of a Runx2 protein in a cell comprises contacting an osteoblast with DHT (dihydrotestosterone), thereby reducing the activity of the Runx2 protein in the osteoblast. The inhibition of the activity of the Runx2 protein by an AR protein in the osteoblast is increased by DHT.

The invention also features a method of identifying a candidate compound for modulating the activity of a Runx2 protein in a cancer cell. The method comprises providing a cancer cell containing a Runx2 protein and an AR protein, contacting the cell with a test compound, and determining the inhibition of the activity of the Runx2 protein by the AR protein in the cell. If the inhibition of the activity of the Runx2 protein by the AR protein in the cell is different from that before the contacting step, it indicates that the test compound is a candidate for modulating the activity of the Runx2 protein in the cancer cell.

The invention further features a method of modulating the activity of a Runx2 protein in a cancer cell. The method comprises providing a cancer cell containing a Runx2 protein and an AR protein, and contacting the cell with a compound identified according to the method described above.

More specifically, a method of modulating the activity of a Runx2 protein in a cancer cell comprises contacting a cancer cell with DHT, thereby modulating the activity of the Runx2 protein in the cell. The inhibition of the activity of the Runx2 protein by an AR protein in the cell is modulated by DHT.

In addition, the invention provides a method of detecting the interaction between a Runx2 protein and an AR protein. The method comprises providing a system containing an AR protein and a Runx2 protein, the AR protein including a DBD and the Runx2 protein including a Runt domain and a PST domain; and detecting the interaction between the DBD of the AR protein and the Runx2 protein, or the interaction between the AR protein and the Runt or PST domain of the Runx2 protein.

Moreover, the invention provides an isolated polypeptide comprising an amino acid sequence at least 95% identical to amino acids 87-507, 1-353 and 376-507, 1-242 and 376-507, or 87-233 and 375-507 of a human Runx2 protein. The polypeptide has 280-506 amino acids in length. In some embodiments, the polypeptide consists of an amino acid sequence at least 95% identical to amino acids 87-507, 1-353 and 376-507, 1-242 and 376-507, or 87-233 and 375-507 of the human Runx2 protein.

The invention also provides an isolated polypeptide comprising an amino acid sequence at least 95% identical to amino acids 176-596, 1-442 and 465-596, 1-331 and 465-596, or 176-322 and 464-596 of a mouse Runx2 protein. The polypeptide has 280-595 amino acids in length. In some embodiments, the polypeptide consists of an amino acid sequence at least 95% identical to amino acids 176-596, 1-442 and 465-596, 1-331 and 465-596, or 176-322 and 464-596 of the mouse Runx2 protein.

The invention further provides a method of modulating the interaction between a Runx2 protein and an AR protein. The method comprises providing a system containing a Runx2 protein and an AR protein and contacting the system with a polypeptide of the invention, thereby modulating the interaction between the Runx2 protein and the AR protein. The inhibition of the activity of the Runx2 protein by the AR protein is modulated by the polypeptide.

Also within the invention is a method of determining whether a subject is suffering from or at risk for developing a bone disorder or cancer. The method comprises detecting the inhibition of the activity of the Runx2 protein by the AR protein in a subject. If the inhibition of the activity of the Runx2 protein by the AR protein in the subject is decreased compared to a control level, it indicates that the subject is likely to be suffering from or at risk for developing a bone disorder or cancer.

In another aspect, the invention features a method of identifying a candidate compound for reducing the activity of a Runx1 protein in a cell. The method comprises providing a system containing a Runx1 protein and an AR protein, contacting the system with a test compound, and determining the inhibition of the activity of the Runx1 protein by the AR protein in the system. If the inhibition of the activity of the Runx1 protein by the AR protein in the system is increased compared to that before the contacting step, it indicates that the test compound is a candidate for reducing the activity of the Runx1 protein in a cell.

In some embodiments, the AR protein contains an NTD (N-terminal domain) and a LBD (ligand-binding domain).

In yet another aspect, the invention features a method of reducing the activity of a Runx1 protein in a cell. The method comprises providing a system containing a Runx1 protein and an AR protein, and contacting the system with a compound identified according to the method described above.

More specifically, a method of reducing the activity of a Runx1 protein in a cell comprises contacting an osteoblast with DHT, thereby reducing the activity of the Runx1 protein in the osteoblast. The inhibition of the activity of the Runx1 protein by an AR protein in the osteoblast is increased by DHT.

The invention also features a method of identifying a candidate compound for modulating the activity of a Runx1 protein in a cancer cell. The method comprises providing a cancer cell containing a Runx1 protein and an AR protein, contacting the cell with a test compound, and determining the inhibition of the activity of the Runx1 protein by the AR protein in the cell. If the inhibition of the activity of the Runx1 protein by the AR protein in the cell is different from that before the contacting step, it indicates that the test compound is a candidate for modulating the activity of the Runx1 protein in the cancer cell.

The invention further features a method of modulating the activity of a Runx1 protein in a cancer cell. The method comprises providing a cancer cell containing a Runx1 protein and an AR protein, and contacting the cell with a compound identified according to the method described above.

More specifically, a method of modulating the activity of a Runx1 protein in a cancer cell comprises contacting a cancer cell with DHT, thereby modulating the activity of the Runx1 protein in the cell. The inhibition of the activity of the Runx1 protein by an AR protein in the cell is modulated by DHT.

In addition, the invention provides a method of detecting the interaction between a Runx1 protein and an AR protein. The method comprises providing a system containing an AR protein and a Runx1 protein, the AR protein including an NTD and a LBD; and detecting the interaction between the NTD or the LBD of the AR protein and the Runx1 protein.

Moreover, the invention provides an isolated polypeptide comprising an amino acid sequence at least 95% identical to amino acids 1-539 and 648-917 of a human AR protein. The polypeptide has 809-916 amino acids in length. In some embodiments, the polypeptide consists of an amino acid sequence at least 95% identical to amino acids 1-539 and 648-917 of the human AR protein.

The invention further provides a method of modulating the interaction between a Runx1 protein and an AR protein. The method comprises providing a system containing a Runx1 protein and an AR protein, and contacting the system with a polypeptide of the invention, thereby modulating the interaction between the Runx1 protein and the AR protein. The inhibition of the activity of the Runx1 protein by the AR protein is modulated by the polypeptide.

Also within the invention is a method of determining whether a subject is suffering from or at risk for developing cancer, a bone disorder, or a disorder of the immune system (e.g., an autoimmune disorder). The method comprises detecting the inhibition of the activity of the Runx1 protein by the AR protein in a subject. If the inhibition of the activity of the Runx1 protein by the AR protein in the subject is decreased compared to a control level, it indicates that the subject is likely to be suffering from or at risk for developing cancer, a bone disorder, or a disorder of the immune system (e.g., an autoimmune disorder).

The above-mentioned and other features of this invention and the manner of obtaining and using them will become more apparent, and will be best understood, by reference to the following description, taken in conjunction with the accompanying drawings. The drawings depict only typical embodiments of the invention and do not therefore limit its scope.

### BRIEF DESCRIPTION OF THE FIGURES

FIG. 1. ERα inhibits Runx2. (A,B) COS7 cells were transiently transfected with the Runx reporter 6XOSE2-luc (firefly) along with expression vectors encoding human Runx2 (A), Runx1 (B), ERα, and/or ERβ as indicated. Cells were treated with DHT, E2, Dex (10 nM each), or ethanol vehicle (0.01%) for 24 h and subjected to luciferase assay as describe in the Materials and Methods section in Example I. (C) COS7 cells were transiently transfected with an ER reporter (ERE-luc) along with an expression vector encoding either ERα or ERβ and treated for 24-hr with E2, followed by the luciferase assay. In each experiment, the firefly luciferase results were corrected for the expression of a co-transfected CMV-renilla luciferase construct (internal control), except for Panel A, where the renilla luciferase values are shown in the inset graph. The immunoblot in A shows that Runx2 expression was not inhibited in the presence of ERα and its ligand. All data are presented as mean values±SEM, with n=4 dish replicates of a representative experiment, repeated at least 3 times.

FIG. 2. Functional mapping of ERα substructures and the dissociation between ERα-mediated transcriptional activation and Runx2 repression. (A) Schematic illustration showing full-length and ERα fragments that were transiently expressed in COS7 cells. The NTD, DBD and LBD fragments were FLAG-tagged to facilitate immunoblot detection shown in the inset of F. (B) Immunoblot analysis of cells transfected with the indicated constructs using antibodies against ERα NTD (sc-7207, left blot) or ERα LBD (sc-787, right blot). (C-F) COS7 cells were transiently transfected with either the 6XOSE2-luc (C,E) or the ERE-luc reporter (D,F), along with the indicated expression plasmids. Luciferase activity was measured 24 h after treatment with either ethanol (0.01%) or E2 (10 nM). All data are presented as mean values±SEM, with n=4 dish replicates of a representative experiment, repeated at least 3 times.

FIG. 3. Interaction between ERα and Runx2 domain in Co-IP and GST pull-down assays. (A) COS7 cells were transiently transfected with plasmids encoding each of the specified ERα fragments and Runx2, followed by 24-hr treatment with either ethanol or 10 nM E2. The ERα and its fragments were detected in either whole cell extracts as input, or IgG or Runx2 immunoprecipitates. (B) Schematic diagram of full-length (FL) Runx2 and fragments transcribed and translated in vitro. The scheme at the top depicts the three Runx2 domains. Boxes with 1, 2, 3 or N mark the positions of the respective activation domains and the NMTS. The thick line above the PST domain represents the surface interacting with ERα. (C) Coomasie stained SDS-PAGE of the bacterially expressed and purified GST or GST fusion proteins used as baits in the pull-down assays. (D, E) A mixture of the indicated radiolabeled Runx2 fragments was incubated with the depicted GST-fusion proteins used as baits. The positive control GST-CBFβ was used as a bait for the Runt domain. The autoradiograph shows the fragments pulled down by the indicated baits.

FIG. 4. Immunofluorescence of ERα and Runx2. COS7 cells were transiently transfected with ERα and Runx2, and treated for 24 h with ethanol or E2 (10 nM). (A-C) ERα (red) and Runx2 (green) were visualized using confocal microscopy as described in Materials and Methods in Example I. Co-localization (yellow in A) is demonstrated by surface plots (B) and by Red/Green profiles (C). (D) Ten cells were randomly selected from each of a set of four untreated and four treated cultures, and co-localization was quantified and plotted as Mean±SEM. *p=8.3×10⁻⁸.

FIG. 5. Developmental stage-specific inhibition of Runx2 by E2 in osteoblasts. (A) MC3T3-E1 cells were treated with ethanol or E2 (10 nM) and the presence of ERα in Runx2 immuno-complexes was examined as described in FIG. 3A. (B-F) MC3T3-E1 cells stably transfected with the 6XOSE2-luc Runx2 reporter were subjected to differentiation conditions and treated with ethanol (white bars) or E2 (10 nM; black bars) commencing at confluence. Levels of the indicated mRNAs were measured on days 4, 11 and 18 by RT-qPCR as described in Material and Methods in Example I. Data were corrected for the expression of ribosomal protein L10A, which itself did not significantly change during culture progression or in response to E2. RANKL was not expressed on day 4, and the low expression of osteocalcin and luciferase on this day are shown in the respective insets (Mean±SD; n=3).

FIG. 6. Various effects of SERM-bound ERα on Runx2. (A) Co-IP assays were performed as in FIG. 3A after treatment of COS7 cells with 100 nM OHT or 100 nM ICI 182780. (B, C) COS7 cells were transfected with Runx2 and its 6XOSE2-luc reporter (B), or with ERE-luc (C) along with expression vectors coding for the indicated ER isoforms or fragments. Cells were treated for 24 h with ethanol, E2 (10 nM), OHT (100 nM), ICI 182780 (100 nM), or combinations thereof, and then subjected to luciferase assays. (D-F) Three breast cancer cell lines, MDA-MB-231 (D), T47D (E) and MCF7 (F) were transfected with the 6XOSE2-luc (left) or the ERE-luc (right) reporter, along with expression vectors for ERα (D), Runx2 (E), or empty vector control, and treated with 10 nM E2, 100 nM OHT or 100 nM ICI 182780 as indicated. All data are presented as mean values±SEM, with n=4 dish replicates of a representative experiment, repeated at least 3 times.

FIG. 7. Meta-analysis of the correlation between expression of ERα and Runx2 target genes in breast cancer biopsies. (A, B) Scatter plots of the expression of MCM5 (A) or pS2 (B) versus ERα in 286 beast cancer biopsies analyzed previously (39). (C) Correlation coefficients between the expression of ERα and each of 41 Runx2 target genes based on meta-analysis of 779 breast cancer biopsies described in three published databases (42-44). Each of the correlation coefficients was significant with p<10⁻⁴. (D) Expression levels of the Runx2 target genes in one cohort of 286 breast cancer biopsies (39) was subjected to an unsupervised cluster analysis, resulting in two major branches of tumor samples designated in the heatmap as 1 and 2. The expression levels of ERα and 4 ERα target genes in each of the 286 biopsies are represented as a heatmap on the right.

FIG. 8. ERα inhibits mouse Runx2. The effects of ERα and ERβ on 6XOSE2-luc were determined in the presence of mouse Runx2. The experimental design is the same as for human Runx2 (FIG. 1A).

FIG. 9. Runx2 does not influence ERα's transcriptional activation activity. The classical transcriptional activation activity of ERα was assessed as in FIG. 1C in the presence and absence of Runx2, as indicated.

FIG. 10. Direct interaction of ERα-LBD with Runx2 in superphysiological conditions. (A) The indicated domains of ERα were produced in BL21DE-3 E. Coli as GST fusion proteins and used to pull down ³⁵S-labeled Runx2 transcribed and translated in reticulocyte lysates. The GST-LBD was also tested in the presence of 1 μM E2 as indicated. (B) SDS-PAGE and Coomassie blue staining of the GST and GST fusion proteins used in Panel B.

FIG. 11. Correlation between expression of ERα and Runx2 target genes in breast cancer biopsies. (A, B) Scatter plots of the expression of MCM5 (A) or pS2 (B) versus ERα in 295 beast cancer biopsies analyzed (1). (C) Expression levels of the Runx2 target genes in the 295 breast cancer biopsies was subjected to an unsupervised cluster analysis, resulting in two major branches designated in the heatmap as 1 and 2. The expression levels of ERα and 4 ERα target genes in each of the 295 biopsies are presented on the right.

FIG. 12. Correlation between expression of ERα and Runx2 target genes in breast cancer biopsies. (A, B) Scatter plots of the expression of MCM5 (A) or pS2 (B) versus ERα in 198 beast cancer biopsies analyzed (2). (C) Expression levels of the Runx2 target genes in the 198 breast cancer biopsies was subjected to an unsupervised cluster analysis, resulting in two major branches designated in the heatmap as 1 and 2. The expression levels of ERα and 4 ERα target genes in each of the 198 biopsies are presented on the right.

FIG. 13. AR represses Runx2 independently of its transcriptional activation property. As COS7 cells were transiently transfected in 96 well format with the Runx2 reporter 6XOSE2-Luc (firefly luciferase) along with expression vectors encoding human Runx2, AR, and GR as indicated. Cells were treated for 24 hrs with DHT, DEX, or vehicle and subjected to luciferase assay. The dotted line indicates the basal level of luciferase activity in the absence of Runx2, which was not affected by any of the receptors or ligands when present individually. B. Parallel sets of COS7 cell cultures were transfected with either the AR reporter MMTV-Luc or the Runx2 reporter 6XOSE-Luc, along with 1 ng each of AR and Runx2 expression plasmids. Cells were treated with the indicated DHT concentrations for 24 hrs, followed by luciferase assay. Concentrations of DHT required for 50% Runx2 repression and 50% AR activation were interpolated from the curves as shown by the dotted lines. C. Block diagram at the top depicts the AR-NTD, -DBD, and -LBD, as well as the AF1 sequence within the NTD and the position of the A573D mutation in the DBD. The two zinc finger motifs constituting the AR-DBD are indicated by Z1 and Z2. Bar graphs represent luciferase assays, in which the reporters OSE2-Luc (left panel) and MMTV-Luc (right panel), were transfected with expression plasmids encoding wild type AR (WT) or a mutant AR lacking the AF1 domain (ΔAF1) as indicated. Insert in the right panel represents immunoblot using the anti-AR antibody. Results were corrected for expression of the internal control CMV-Renilla luciferase and presented as mean relative light units (RLU)±SEM, with n=4 dish replicates of a representative experiment, repeated at least 3 times.

FIG. 14. DNA-binding domain of AR mediates its interaction with Runx2. A. Whole cell extracts from COS7 cells co-expressing AR and Flag-Runx2 were immunoprecipitated using anti-Flag or non-specific (IgG) antibodies and the immunoprecipitates were analyzed by Western blotting with anti-AR and anti-Runx2 antibodies. B. GST and the indicated AR-derived GST-fusion proteins were produced in Escherichia coli and 10 μg of each was subjected to SDS-PAGE and Coomasie blue staining. C. Baits shown in B were used to pull-down ³⁵S-labeled murine Runx2 produced using reticulocyte lysates. SDS-PAGE and autoradiography show the exclusive interaction between GST-AR-DBD and Runx2. D. ³⁵S-Runx2 was pulled-down using GST and GST-AR-DBD as baits in the presence of the indicated mM concentrations of NaCl. E. Coomasie blue stained SDS-PAGE of intact or the indicated fragments of AR-DBD fused to GST. F. The baits shown in E were used to pull-down ³⁵S-Runx2. SDS-PAGE and autoradiography show that only the intact AR-DBD interacted with Runx2. G. Coomasie blue-stained SDS-PAGE showing WT and mutant AR-DBD used as GST-fusion baits to pull-down ³⁵S-Runx2. H. GST pull-down assay using the baits shown in G and ³⁵S-Runx2. AR-DBD^{A573D} did not interact with Runx2. Input lanes (In) represent 5% of the prey used for each pull-down assay. I. Luciferase assay of COS7 cells transiently transfected in a 96-well format with the 6XOSE-Luc reporter and expression vector for human Runx2 alone or along with those encoding WT AR, AR^{A573D}, Flag-AR-DBD, or Flag-AR-NTD as indicated. Below the bar diagram are the western blots showing comparable expression of AR^{A573D} and WT AR(N-20 antibody) and of AR-DBD and AR-NTD (α-Flag antibody). The dotted line indicates the background luciferase activity in the absence of Runx (Mean±SEM, with n=4 dish replicates of a representative experiment, repeated at least 3 times).

FIG. 15. Colocalization and evidence for interaction between AR and Runx2 in living cells. A. COS7 cells were transfected with the plasmid/s indicated at the top, immunostained and analyzed by confocal microscopy to visualize the AR (red) and/or Runx2 (green). B. Runx2-GFP fusion protein was expressed in COS7 cells alone or together with WT AR or AR^{A573D} in the presence of DHT or vehicle. Curves represent FRAP relative to the pre-photobleaching intensity.

FIG. 16. AR inhibits Runx2 in PCa cells. A. PC3 cells were transfected with AR-encoding plasmid or the empty vector as control, immunostained and then analyzed by confocal microscopy to visualize the AR (red) and/or the endogenous Runx2 (green). B. PC3 cell that had been stably transduced with lentiviruses encoding AR or GFP were transiently transfected with the MMTV-Luc (left) or 6XOSE2-Luc (middle) reportersk, followed by luciferase assay, or, they were subjected to RT-qPCR analysis of the OC gene (right). White and black bars represent 24 hrs treatments with DHT or vehicle, respectively. Mean±SEM, n=4. C. Primary prostate cancer tumors resected from 23 untreated patients (red diamonds) and 17 patients undergoing androgen ablation therapy (AAT; black squares) were previously subjected to comprehensive gene expression analysis (59). The negative correlation between OC and PSA mRNA levels in these tumors is demonstrated by best-fit linear regression of the respective values from the untreated patients (dashed line) and those undergoing AAT (solid line). The calculated R values and the levels of statistical significance assigned by the Wilcoxon signed-rank test are depicted in the inset.

FIG. 17. Differential intra-cellular localization of AR in osteoblastic cell lines correlates with the repression of Runx2. A. ROS 17/2.8 and SaOS-2 cells were transiently transfected with the 6XOSE2-Luc reporter and treated for 24 hrs with DHT or vehicle, followed by luciferase assay. B. Confocal microscopy images of immunostained ROS 17/2.8 and SaOS-2 cells showing the intracellular distribution of AR (red) and Runx2 (green) after 24 hrs of treatment with DHT or vehicle. C. Differentiating MC3T3E-1 cells stably transfected with the 6XOSE2-Luc reporter were treated with DHT or vehicle commencing at confluence (day 0), and levels of the indicated mRNAs were measured on day 1 and day 9. Data were corrected for ribosomal protein L10A mRNA, which itself did not significantly change in response to DHT (Mean±SEM; n=3). D. Day-1 and Day-9 MC3T3E-1 cultures were treated for 24 hrs with DHT or vehicle, and subjected to confocal microscopy for visualization of AR (red) and Runx2 (green) after immunostaining with the respective antibodies. Staining with 4′,6-diamidino-2-phenylindole (DAPI) demarcates the cell nucleus. E. Co-IP assay of day-9 MC3T3-E1 cells that were treated for 24 hrs with DHT or vehicle. Immunoprecipitates were obtained using Runx2 specific or non-specific IgG antibodies, and were subjected, along with 5% of the input, to Western blot analysis for the detection of AR and Runx2. Data are representative of at least three independent experiments.

FIG. 18. Mapping of Runx2 sequences required for binding AR-DBD. Shown at the top is a block diagram of Runx2 with the QA, Runt and PST domains. Depicted within the PST domain are the nuclear localization signal (NLS), activation domain (AD), and the overlapping nuclear matrix targeting signal (NMTS) and SMAD interacting domain (SMID). The black thick line just below the block diagram represents the minimal Runx2 sequences required for binding AR-DBD as defined in present study. Thick grey lines below represent a series of ³⁵S-Runx2 fragments used as preys in pull-down assays with GST or GST-AR-DBD as baits. The results of these assays are shown to the right of each fragment, with the interaction scored as strong (+++) weak (+), or absent (−).

FIG. 19. AR-DBD diminishes Runx2's interaction with its OSE2 target in vitro and in vivo. A. EMSA was performed using ³²P-labeled OSE2 probe and MC3T3-E1 whole cell extract as the source of Runx2. Where indicated, the binding reaction also contained purified GST, GST-AR-DBD, GST-AR-DBD^{A573D} (see FIG. 14G), or anti-Runx2 specific antibodies, either native (n) or denaturated by boiling (b). The relative amounts of the indicated proteins added to the binding reaction are depicted by + or +++ (see Materials and Methods in Example II). Arrow indicates Runx2/OSE2 complex. B. Differentiating Day-9 MC3T3E-1 cultures were treated for 4 hrs with DHT or vehicle and subjected to ChIP assay using anti-Runx2 or non-specific IgG antibodies. Occupancy was quantified by qPCR of an OC promoter fragment containing the OSE2 site or a fragment of the insulin promoter as control.

FIG. 20. AR inhibition by Runx Proteins. AR and increasing concentrations of Runx1 (A) and Runx2 (B) expression vectors were transiently transfected in Cos7 cells as indicated. AR activity was assessed on three different luciferase reporter constructs: Probasin, MMTV and PSA540.

FIG. 21. AR mutants and Runx protein inhibition. A, B. Transiently transfected COS7 cells show inhibition of Runx2 and Runx1 proteins using different AR mutant constructs as indicated and assessed using 6XOSE2-luciferase reporter. C. Activity of AR mutants constructs themselves as assessed in transiently transfected COS7 cells with a PSA 540-luciferase reporter.

FIG. 22. AR activation versus Runx2 inhibition. The percent activation of AR was compared with the percent inhibition of Runx2 activity as a function of DHT concentration and using luciferase reporter constructs of AR (MMTV-luciferase) and Runx2 (6XOSE2-luciferase) in transiently transfected COS7 cells. The ED₅₀ for AR activation was 3.19 nM and for Runx2 inhibition was 0.27 nM.

FIG. 23. Runx proteins and AR interaction in vivo and in vitro. A. Immunoprecipitation of indicated Runx proteins and immunoblot against indicated AR constructs all transfected in COS7 cells. B. GST-pull down of in vitro translated ^{s}35-labeled Runx1 and Runx2 with BCL21 translated GST-NTD, DBD, or LBD. C. Confocal analyses of transfected AR and Runx proteins under the indicated conditions.

FIG. 24. Interaction between AR and Runx proteins in PC3 cells that express endogenous Runx proteins. A. EMSA assessing Runx protein activity using an OSE2 ³²P-labeled probe. Whole cell extracts from PC3 cells were shifted and supershifted with Runx1 and Runx2 antibodies respectively and successfully competed with WT 10× and 100×OSE2 unlabeled probe. B. Endogenous Runx luciferase activity was assayed using a 6XOSE2-luciferase reporter under the above conditions, and was inhibited with transfected AR after 10 nM DHT treatment. C. Confocal of endogenous Runx (fluorescein) proteins and AR (rhodamine) under transfected or untransfected AR conditions.

FIG. 25. Androgens, but not Estrogens inhibit Runx1. The ability of sex steroids and their receptors to repress Runx1 was determined by transient transfection assays of COS7 cells as described earlier for Runx2. Inset: Western analysis shows that AR+DHT do not inhibit Runx1 protein levels.

FIG. 26. Effect of raloxifene on Runx2 activity.

FIG. 27. Comparison of human and mouse Runx2.

### DETAILED DESCRIPTION OF THE INVENTION

Runx2 is a master osteoblast transcription factor playing pivotal roles in skeletal development and homeostasis. In humans, Runx2 haplotypes contribute to variations in bone mass. Runx1, which is expressed in osteoblasts and shares similar DNA-binding properties with Runx2, has been implicated in bone metabolism as well. Sex steroid hormones and their receptors (SHRs) also play critical roles in bone health and disease, and are targets for existing and developing drugs that affect bone mass and fragility either positively or negatively. The proskeletal effects of sex steroids are mediated by anabolic effects in osteoblasts, but more importantly by attenuating bone resorption. The anti-resorptive effects of sex steroids are attributable to both direct pro-apoptotic action in osteoclasts and indirect inhibition of bone turnover via poorly understood mechanisms in osteoblasts and other mesenchymal cells. We found that the activated estrogen receptor α (ERα) and the androgen receptor (AR) each inhibits Runx2, and that AR, but not ERα, inhibits Runx1 as well. These inhibitory activities are important in light of recent data from a1(I) collagen-Runx2 transgenic mice, indicating that Runx2 must be restrained in order to keep bone turnover in check and prevent osteoporosis.

Therefore, one object is to investigate in depth the physical interactions between Runx proteins and SHRs, the mechanisms mediating the resulting inhibition of Runx2 and/or Runx1, and the in vivo physiological implications by analyses of recombinant and transiently expressed proteins, as well as the endogenous SHR and Runx proteins in osteoblasts, including their associations with each other, with co-regulators, and with genomic Runx targets. More specifically, one object of the invention is to dissect the functional and molecular interactions between ERα and Runx2. Another object of the invention is to dissect the functional and molecular interactions between AR and Runx2, as well as between AR and Runx1. Based on the data presented below, it is believed that both similar and unique features for each of these interactions exist. Still another object of the invention is to test the ability of estrogens and androgens to correct the hyper-osteoclastogenic phenotype of osteoblasts over-expressing Runx2 in vivo and when co-cultured with osteoclasts. Yet another object of the invention is to address novel mechanisms of action of selective estrogen receptor modulators (SERMs). Like estradiol, SERMs promote a physical interaction between ERα and Runx2. However, SERMs elicit different functional outcomes, possibly explaining the variable skeletal effects of these drugs. The present invention provides novel insights into the regulation of skeletal metabolism by sex hormones, and reveals commonalties and differences between the genders at the molecular level, which decipher cryptic mechanisms of action of existing SERMs and support the rationale for the development of novel ones, based on their influence on Runx proteins.

### Screening Assays

The invention provides methods (also referred to herein as “screening assays”) for identifying test compounds (e.g., proteins, peptides, peptidomimetics, peptoids, antibodies, small molecules, or other drugs) that modulate (i.e., induce, increase, or decrease) the inhibition of the Runx2 activity by ERα or AR, or the inhibition of the Runx1 activity by AR. Compounds thus identified can be used to treat conditions characterized by over-activity or under-activity of Runx2 or Runx1 as described below.

The test compounds of the present invention can be obtained using any of the numerous approaches in combinatorial library methods known in the art. Such libraries include: peptide libraries, peptoid libraries (libraries of molecules having the functionalities of peptides, but with a novel, non-peptide backbone that is resistant to enzymatic degradation; see, e.g., Zuckernann et al. (1994) J Med Chem 37:2678-85); spatially addressable parallel solid phase or solution phase libraries; synthetic libraries obtained by deconvolution or affinity chromatography selection; and the “one-bead one-compound” libraries Compounds in the last three libraries can be peptides, non-peptide oligomers, or small molecules (Lam (1997) Anticancer Drug Des 12:145). Exemplary test compounds include SERMs (see, e.g., Saji and Kuroi (2008) Breast Cancer 15:262-9; Jordan and O'Malley (2007) J Clin Oncol 25:5815-24) and SARMs (see, e.g., Gao and Dalton (2007) Drug Discov Today 12 (5-6):241-8).

Examples of methods for the synthesis of molecular libraries can be found in the art, for example, in: DeWitt et al. (1993) PNAS USA 90:6909; Erb et al, (1994) PNAS USA 91:11422; Zuckermann et al. (1994) J Med Chem 37:2678; Cho et al. (1993) Science 261:1303; Carrell et al. (1994) Angew Chem Int Ed Engl 33:2059; Carell et al. (1994) Angew Chem Int Ed Engl 33:2061; and Gallop et al. (1994) J Med Chem 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. 5,223,409), plasmids (Cull et al. (1992) PNAS USA 89:1865-1869), or phages (Scott and Smith (1990) Science 249:386-390; Devlin (1990) Science 249:404-406; Cwirla et al. (1990) PNAS USA 87:6378-6382; Felici (1991) J Mol Biol 222:301-310; and Ladner supra.).

### Runx2 and ERα

One screening method of the invention involves providing a system containing a Runx2 protein and an ERα protein, contacting the system with a test compound, and determining the inhibition of the activity of the Runx2 protein by the ERα protein in the system. If the inhibition of the activity of the Runx2 protein by the ERα protein in the system is increased (including induced) compared to that before the contacting step, it indicates that the test compound is a candidate for reducing the activity of the Runx2 protein in a cell.

To identify compounds that increases the inhibition of the activity of the Runx2 protein by the ERα protein, a reaction mixture containing Runx2 (or an ERα-binding portion or variant of it) and ERα (or a Runx2-binding portion or variant of it) is prepared under conditions and for a time sufficient to allow the interaction between Runx2 and ERα.

Runx2 and ERα are well known in the art. For example, human Runx2 and ERα can be found in GenBank accession numbers NG_{—}008020 and NP_{—}000116, respectively. Mouse Runx2 can be found in GenBank accession number NM_{—}009820.2. The ERα-binding portion of the human Runx2 is located in the PST domain (amino acids 215-507) including amino acids 328-425. The ERα-binding portion of the mouse Runx2 is located in the PST domain (amino acids 304-596) including amino acids 417-514. The Runx2-binding portion of human ERα is located in the DBD (amino acids 181-265).

A “system” of the invention may be a cell system or a cell-free system, where cell extracts or purified or synthetic components (e.g., proteins) may also be used. For example, an osteoblastic cell expressing both Runx2 and ERα may be employed. Osteoblastic is a characteristic of a cell that resembles that of an osteoblast. A cell is considered osteoblastic when it expresses gene/s that typify the osteoblast phenotype, such as osteocalcin, bone sialoprotein, and alkaline phosphatase, and/or if it lays down a collagenous extracellular matrix and induces mineralization of the extracellular matrix. Osteoblastic cells include primary osteoblasts isolated from bone, cell lines isolated from normal bone tissue or osteosarcoma tissue, and mesenchymal cells that become similar to osteoblasts after being contacted with reagents such as a Runx2 expression vector or bone morphogenetic proteins. Other cells that express endogenous or exogenous Runx2 and ERα may also be employed.

The test compound can be initially included in the reaction mixture, or can be added at a time subsequent to the addition of Runx2 and ERα. Control reaction mixtures are incubated without the test compound.

The test compound may or may not activate the ERα protein, i.e., induce any structural or functional change in the protein, including increasing the transcription of ERα target genes. The ERα protein may also be transcriptionally incompetent, i.e., incapable of increasing the transcription of ERα target genes.

The inhibition of the activity of the Runx2 protein by the ERα protein is then determined. “Runx2 activity” refers to the ability of the Runx2 protein to increase the transcription of a target gene, either an endogenous gene in the cell or a foreign gene introduced into the cell to report on the ability of Runx2 to increase transcription.

The activity of Runx2 can be determined by measuring the product of an endogenous Runx2 target gene or the foreign Runx2 reporter gene. For example, if the endogenous gene is osteocalcin, then the activity of Runx2 can be determined based on measuring osteoclacin mRNA levels. In this case, to attribute a change in osteocalcin mRNA level to a change in Runx2 activity, it helps to show that the change no longer occurs after Runx2 has been depleted. In another example, if the foreign gene is a fusion gene containing Runx2 binding sites and a luciferase reporter gene, then the activity of Runx2 can be determined by measuring the light emitted by the luciferase protein.

The inhibition of the activity of the Runx2 protein by the ERα protein can be determined by comparing the activity levels of the Runx2 protein in the presence and absence of a compound that induces the inhibition of the activity of the Runx2 protein by the ERα protein, or the activity levels of the Runx2 protein in the presence and absence of the ERα protein. If the inhibition of the activity of the Runx2 protein by the ERα protein in the system is increased compared to that before the contacting step, it indicates that the test compound is a candidate for reducing the activity of the Runx2 protein in a cell.

Another screening method of the invention involves providing a cancer cell containing a Runx2 protein and an ERα protein, contacting the cell with a test compound, and determining the inhibition of the activity of the Runx2 protein by the ERα protein in the cell. If the inhibition of the activity of the Runx2 protein by the ERα protein in the cell is different from that before the contacting step, it indicates that the test compound is a candidate for modulating the activity of the Runx2 protein in the cancer cell. This method may be carried out similarly to that described above.

Cancer is a class of diseases in which a group of cells display uncontrolled growth (division beyond the normal limits), invasion (intrusion on and destruction of adjacent tissues), and sometimes metastasis (spread to other locations in the body via lymph or blood). An early stage cancer is when the tumor is confined to the organ of origin; a late stage cancer is when the tumor has invaded an adjacent tissue or metastasized to other organ sites. Any cancer cell that expresses endogenous or exogenous Runx2 and ERα may be employed in this method. Preferably, the cancer cell is a breast, leukemia, colorectal, or ovarian cancer cell.

### Runx2 and AR

One screening method of the invention involves providing a system containing a Runx2 protein and an AR protein, contacting the system with a test compound, and determining the inhibition of the activity of the Runx2 protein by the AR protein in the system. If the inhibition of the activity of the Runx2 protein by the AR protein in the system is increased (including induced) compared to that before the contacting step, it indicates that the test compound is a candidate for reducing the activity of the Runx2 protein in a cell.

Another screening method of the invention involves providing a cancer cell containing a Runx2 protein and an AR protein, contacting the cell with a test compound, and determining the inhibition of the activity of the Runx2 protein by the AR protein in the cell. If the inhibition of the activity of the Runx2 protein by the AR protein in the cell is different from that before the contacting step, it indicates that the test compound is a candidate for modulating the activity of the Runx2 protein in the cancer cell. Any cancer cell that expresses endogenous or exogenous Runx2 and AR may be employed in this method. Preferably, the cancer cell is a prostate, leukemia, lung, or testicular cancer cell.

These methods may be practiced similarly to those described for Runx2 and ERα above with modifications specific for Runx2 and AR. In general, the AR protein should be used instead of the ERα protein.

AR is well known in the art. For example, human AR can be found in GenBank accession number NM_{—}000044. The AR-binding portion of the human Runx2 is located in the Runt domain (amino acids 88-214) and the PST domain (amino acids 215-507). The AR-binding portion of the mouse Runx2 is located in the Runt domain (amino acids 177-303) and the PST domain (amino acids 304-596). The Runx2-binding portion of human AR is located in the DBD (amino acids 540-647).

### Runx1 and AR

One screening method of the invention involves providing a system containing a Runx1 protein and an AR protein, contacting the system with a test compound, and determining the inhibition of the activity of the Runx1 protein by the AR protein in the system. If the inhibition of the activity of the Runx1 protein by the AR protein in the system is increased (including induced) compared to that before the contacting step, it indicates that the test compound is a candidate for reducing the activity of the Runx1 protein in a cell.

Another screening method of the invention involves providing a cancer cell containing a Runx1 protein and an AR protein, contacting the cell with a test compound, and determining the inhibition of the activity of the Runx1 protein by the AR protein in the cell. If the inhibition of the activity of the Runx1 protein by the AR protein in the cell is different from that before the contacting step, it indicates that the test compound is a candidate for modulating the activity of the Runx1 protein in the cancer cell. Any cancer cell that expresses endogenous or exogenous Runx1 and AR may be employed in this method. Preferably, the cancer cell is a leukemia or prostate cancer cell.

These methods may be practiced similarly to those described for Runx2 and AR above with modifications specific for Runx1 and AR. In general, the Runx1 protein should be used instead of the Runx2 protein.

Runx1 is well known in the art. For example, human Runx1 can be found in GenBank accession number NM_{—}001754. The Runx1-binding portion of human AR is located in the NTD (amino acids 1-539) and the LBD (amino acids 648-917).

### Polypeptides and Nucleic Acids

One aspect of the invention pertains to an isolated polypeptide comprising or consisting of an amino acid sequence at least 95% (e.g., 96%, 97%, 98%, 99%, and 100%) identical to amino acids 88-507, 88-425, 319-507, or 328-425 of a human Runx2 protein. The polypeptide has 98-506 (e.g., 100, 200, 300, 400, and 500) amino acids in length.

Also, the invention pertains to an isolated polypeptide comprising or consisting of an amino acid sequence at least 95% (e.g., 96%, 97%, 98%, 99%, and 100%) identical to amino acids 177-596, 177-514, 408-596, or 417-514 of a mouse Runx2 protein. The polypeptide has 98-595 (e.g., 100, 200, 300, 400, and 500) amino acids in length.

Another aspect of the invention pertains to an isolated polypeptide comprising or consisting of an amino acid sequence at least 95% (e.g., 96%, 97%, 98%, 99%, and 100%) identical to amino acids 87-507, 1-353 and 376-507, 1-242 and 376-507, or 87-233 and 375-507 of a human Runx2 protein. The polypeptide has 280-506 (e.g., 300, 350, 400, 450, and 500) amino acids in length.

Also, the invention pertains to an isolated polypeptide, comprising or consisting of an amino acid sequence at least 95% (e.g., 96%, 97%, 98%, 99%, and 100%) identical to amino acids 176-596, 1-442 and 465-596, 1-331 and 465-596, or 176-322 and 464-596 of a mouse Runx2 protein. The polypeptide has 280-595 (e.g., 300, 350, 400, 450, 500, and 550) amino acids in length.

The invention also pertains to an isolated polypeptide comprising or consisting of an amino acid sequence at least 95% (e.g., 96%, 97%, 98%, 99%, and 100%) identical to amino acids 1-539 and 648-917 of a human AR protein. The polypeptide has 809-916 (e.g., 810, 830, 850, 870, 890, and 910) amino acids in length.

The present invention further pertains to variants which function as either agonists (mimetics) or antagonists of a peptide of the invention. Such variants can be generated by mutagenesis, e.g., discrete point mutation or truncation of the polypeptide. An agonist of the polypeptide can retain substantially the same, or a subset, of the biological activities of the naturally occurring form of the polypeptide. An antagonist of the polypeptide can inhibit one or more of the activities of the naturally occurring form of the polypeptide by, for example, competitively binding to a downstream or upstream member of a cellular signaling cascade which involves the polypeptide. Thus, specific biological effects can be elicited by treatment with a variant of limited function.

In yet another aspect, the invention provides an isolated nucleic acid that encodes a polypeptide of the invention. An “isolated” nucleic acid molecule is one which is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived.

Nucleic acids of the inventor can be chosen for having codons, which are preferred, or non-preferred, for a particular expression system. For example, the nucleic acid can be one in which at least one codon, and preferably at least 10% or 20% of the codons, has been altered such that the sequence is optimized for expression, e.g, in bacterial or mammalian cells.

Variants of a nucleic acid of the invention can be naturally occurring, such as allelic variants (same locus), homologs (different locus), and orthologs (different organism), or can be non-naturally occurring. Non-naturally occurring variants can be made by mutagenesis techniques, including those applied to polynucleotides, cells, or organisms. The variants can contain nucleotide substitutions, deletions, inversions, and insertions. The variations can produce both conservative and non-conservative amino acid substitutions. The term “conservative substitution,” as used herein, denotes the replacement of an amino acid by another biologically similar residue. Examples of conservative substitutions include the substitution of one hydrophobic residue such as isoleucine, valine, leucine, or methionine for another, or the substitution of one polar residue for another, such as the substitution of arginine for lysine, glutamic acid for aspartic acid, or glutamine for asparagine, and the like.

An additional aspect of the invention includes vectors containing a nucleic acid encoding a polypeptide of the invention. As used herein, the term “vector” refers to a nucleic acid molecule capable of transporting another nucleic acid to which it has been linked and can include a plasmid or cosmid vector. The vector can be capable of autonomous replication, or it can integrate into a host DNA.

A vector can include a nucleic acid of the invention in a form suitable for expression of the nucleic acid in a host cell. Preferably the recombinant expression vector includes one or more expression control sequences operatively linked to the nucleic acid sequence to be expressed. Expression control sequences include those which direct constitutive expression of a nucleotide sequence, as well as tissue-specific regulatory and/or inducible sequences. The design of the expression vector can depend on such factors as the choice of the host cell to be transformed, the expression level of the polypeptide desired, and the like.

The expression vectors of the invention can be introduced into host cells to thereby produce the polypeptide, as well as a fusion polypeptide, a mutant form of the polypeptide, and the like. A host cell can be any prokaryotic or eukaryotic cell. A vector DNA can be introduced into host cells via conventional transformation or transfection techniques, including calcium phosphate or calcium chloride co-precipitation, DEAE-dextran-mediated transfection, lipofection, or electroporation.

The host cells of the invention can also be used to produce non-human transgenic animals. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. Transgenic animals are useful for studying the function and/or activity of the polypeptides of the invention and for identifying and/or evaluating modulators of the polypeptides.

A polypeptide or nucleic acid of the invention can be produced by recombinant DNA techniques well known in the art. Alternatively, a polypeptide or nucleic acid of the invention can be synthesized chemically using standard peptide or nucleic acid synthesis techniques.

An “isolated” polypeptide or nucleic acid is substantially free of cellular material or other contaminating proteins or nucleic acids from the cell or tissue source from which the polypeptide or nucleic acid is derived, or substantially free from chemical precursors or other chemicals when chemically synthesized, e.g., a preparation of a polypeptide or nucleic acid may have less than about 30%, 20%, 10%, or 5% (by dry weight) of cellular material, contaminating proteins or nucleic acids, chemical precursors, or other chemicals.

### Treatment Methods

A candidate compound identified using a screening method of the invention, a polypeptide of the invention, or a nucleic acid of the invention (collectively as compounds of the invention) can be used to modulate the inhibition of the Runx2 activity by ERα or AR, or the inhibition of the Runx1 activity by AR in vivo and in vitro, and treat conditions involving Runx2-ERα, Runx2-AR, or Runx1-AR interactions. Exemplary conditions involving Runx2-ERα or Runx2-AR interactions include osteoporosis, breast cancer, prostate cancer, leukemia, colorectal cancer, ovarian cancer, lung cancer, testicular cancer, and cardiovascular disease; exemplary conditions involving Runx1-AR interactions include osteoporosis, leukemia, prostate cancer, a disease of the immune system such as an autoimmune disease, and cardiovascular disease.

One method of reducing the activity of a Runx2 protein in a cell involves the steps of providing a system containing a Runx2 protein and an ERα protein, and contacting the system with a compound of the invention that increases the inhibition of the activity of the Runx2 protein by the ERα protein. For example, the activity of a Runx2 protein in an osteoblast may be reduced by treating the cell with E2, OHT, ICI 182780, or raloxifene.

Another method of reducing the activity of a Runx2 protein in a cell involves the steps of providing a system containing a Runx2 protein and an AR protein, and contacting the system with a compound of the invention that increases the inhibition of the activity of the Runx2 protein by the AR protein. For example, the activity of a Runx2 protein in an osteoblast may be reduced by treating the cell with DHT.

Similarly, a method of reducing the activity of a Runx1 protein in a cell involves the steps of providing a system containing a Runx1 protein and an AR protein, and contacting the system with a compound of the invention that increases the inhibition of the activity of the Runx1 protein by the AR protein. For example, the activity of a Runx1 protein in an osteoblast may be reduced by treating the cell with DHT.

Furthermore, a method of modulating the activity of a Runx2 protein in a cancer cell involves the steps of providing a cancer cell containing a Runx2 protein and an ERα protein, and contacting the cell with a compound of the invention that modulates the inhibition of the activity of the Runx2 protein by the ERα protein. For example, the activity of a Runx2 protein in a cancer cell may be modulated by treating the cell with E2, OHT, ICI 182780, or raloxifene.

Another method of modulating the activity of a Runx2 protein in a cancer cell involves the steps of providing a system containing a Runx2 protein and an AR protein, and contacting the system with a compound of the invention that modulates the inhibition of the activity of the Runx2 protein by the AR protein. For example, the activity of a Runx2 protein in a cancer cell may be modulated by treating the cell with DHT.

Similarly, a method of modulating the activity of a Runx1 protein in a cancer cell involves the steps of providing a system containing a Runx1 protein and an AR protein, and contacting the system with a compound of the invention that modulates the inhibition of the activity of the Runx1 protein by the AR protein. For example, the activity of a Runx1 protein in a cancer cell may be modulated by treating the cell with DHT.

In addition, a method of modulating the interaction between a Runx2 protein and an ERα protein involves the steps of providing a system containing a Runx2 protein and an ERα protein, and contacting the system with a polypeptide of the invention that modulates the interaction between the Runx2 protein and the ERα protein.

A method of modulating the interaction between a Runx2 protein and an AR protein involves the steps of providing a system containing a Runx2 protein and an AR protein, and contacting the system with a polypeptide of the invention that modulates the interaction between the Runx2 protein and the AR protein.

A method of modulating the interaction between a Runx1 protein and an AR protein involves the steps of providing a system containing a Runx1 protein and an AR protein, and contacting the system with a polypeptide of the invention that modulates the interaction between the Runx1 protein and the AR protein.

The invention also provides a method for treating a condition involving Runx2-ERα, Runx2-AR, or Runx1-AR interactions in a subject by administering to a subject in need thereof an effective amount of a compound of the invention.

As used herein, a “subject” refers to a human or animal, including all mammals such as primates (particularly higher primates), sheep, dog, rodents (e.g., mouse or rat), guinea pig, goat, pig, cat, rabbit, and cow. In a preferred embodiment, the subject is a human. In another embodiment, the subject is an experimental animal or animal suitable as a disease model.

A subject to be treated may be identified in the judgment of the subject or a health care professional, which can be subjective (e.g., by opinion) or objective (e.g., reached by determining the activity level of Runx2 or Runx1 as described above).

A “treatment” is defined as administration of a substance to a subject with the purpose to cure, alleviate, relieve, remedy, prevent, or ameliorate a disorder, symptoms of the disorder, a disease state secondary to the disorder, or predisposition toward the disorder.

An “effective amount” is an amount of a compound that is capable of producing a medically desirable result in a treated subject. The medically desirable result may be objective (i.e., measurable by some test or marker) or subjective (i.e., subject gives an indication of or feels an effect).

For treatment of cancer, a compound is preferably delivered systemically, or directly to tumor cells, e.g., to a tumor or a tumor bed following surgical excision of the tumor, in order to treat any remaining tumor cells. For prevention of cancer invasion and metastases, the compound can be administered to, for example, a subject that has not yet developed detectable invasion and metastases.

In some embodiments, polynucleotides (nucleic acids of the invention, or antisense nucleic acids, ribozymes, and siRNAs thereof) are administered to a subject. Polynucleotides can be delivered to target cells by, for example, the use of polymeric, biodegradable microparticle or microcapsule devices known in the art. Another way to achieve uptake of a nucleic acid is using liposomes, prepared by standard methods. The polynucleotides can be incorporated alone into these delivery vehicles or co-incorporated with tissue-specific or tumor-specific antibodies. Alternatively, one can prepare a molecular conjugate composed of a polynucleotide attached to poly-L-lysine by electrostatic or covalent forces. Poly-L-lysine binds to a ligand that can bind to a receptor on target cells. “Naked DNA” (i.e., without a delivery vehicle) can also be delivered to an intramuscular, intradermal, or subcutaneous site. A preferred dosage for administration of polynucleotide is from approximately 10⁶ to 10¹² copies of the polynucleotide molecule.

A compound of the invention can be incorporated into a pharmaceutical composition. Such compositions typically include the compound and a pharmaceutically acceptable carrier. “Pharmaceutically acceptable carriers” include solvents, dispersion media, coatings, antibacterial and antifungal agents, isotonic and absorption delaying agents, and the like, compatible with pharmaceutical administration.

A pharmaceutical composition can be formulated to be compatible with its intended route of administration. See, e.g., U.S. Pat. No. 6,756,196. Examples of routes of administration include parenteral, e.g., intravenous, intradermal, subcutaneous, oral (e.g., inhalation), transdermal (topical), transmucosal, and rectal administration.

It is advantageous to formulate oral or parenteral compositions in dosage unit form for ease of administration and uniformity of dosage. “Dosage unit form,” as used herein, refers to physically discrete units suited as unitary dosages for the subject to be treated, each unit containing a predetermined quantity of an active compound calculated to produce the desired therapeutic effect in association with the required pharmaceutical carrier.

The dosage required for treating a subject depends on the choice of the route of administration, the nature of the formulation, the nature of the subject's illness, the subject's size, weight, surface area, age, and sex, other drugs being administered, and the judgment of the attending physician. Suitable dosages are in the range of 0.01-100.0 mg/kg. Wide variations in the needed dosage are to be expected in view of the variety of compounds available and the different efficiencies of various routes of administration. For example, oral administration would be expected to require higher dosages than administration by intravenous injection. Variations in these dosage levels can be adjusted using standard empirical routines for optimization as is well understood in the art. Encapsulation of the compound in a suitable delivery vehicle (e.g., polymeric microparticles or implantable devices) may increase the efficiency of delivery, particularly for oral delivery.

Toxicity and therapeutic efficacy of a compound or composition of the invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and can be expressed as the ratio of LD₅₀/ED₅₀. Compounds or compositions which exhibit high therapeutic indices are preferred. While compounds or compositions that exhibit toxic side effects may be used, care should be taken to design a delivery system that targets such compounds or compositions to the site of affected tissue in order to minimize potential damage to uninfected cells and, thereby, reduce side effects.

The data obtained from the cell culture assays and animal studies can be used in formulating a range of dosage for use in humans. The dosage of the compounds lies preferably within a range of circulating concentrations that include the ED₅₀ with little or no toxicity. The dosage may vary within this range depending upon the dosage form employed and the route of administration utilized. For any compound or composition to be used in a method of the invention, the therapeutically effective dose can be estimated initially from cell culture assays. A dose may be formulated in animal models to achieve a circulating plasma concentration range that includes the IC₅₀ (i.e., the concentration of a compound which achieves a half-maximal inhibition of symptoms) as determined in cell culture. Such information can be used to more accurately determine useful doses in humans. Levels in plasma may be measured, for example, by high performance liquid chromatography.

A compound or composition of the invention may be used alone, or in combination with other therapeutic agents. The compound or composition of the invention and the other therapeutic agents may be administered simultaneously or sequentially, as mixed or individual dosages.

### Diagnostic Methods

The invention provides diagnostic methods based on the level of the inhibition of the Runx2 activity by ERα or AR, or the inhibition of the Runx1 activity by AR.

One such method involves detecting the inhibition of the activity of the Runx2 protein by the ERα protein in a subject. If the inhibition of the activity of the Runx2 protein by the ERα protein in the subject is decreased compared to a control level, it indicates that the subject is likely to be suffering from or at risk for developing a bone disorder (e.g., osteoporosis), cancer (e.g., breast cancer, prostate cancer, leukemia, colorectal cancer, ovarian cancer, lung cancer, and testicular cancer), or cardiovascular disorder. A control level may be, for example, the level of the inhibition of the activity of the Runx2 protein by the ERα protein in a normal subject.

Another method of the invention involves detecting the inhibition of the activity of the Runx2 protein by the AR protein in a subject. If the inhibition of the activity of the Runx2 protein by the AR protein in the subject is decreased compared to a control level, it indicates that the subject is likely to be suffering from or at risk for developing a bone disorder (e.g., osteoporosis), cancer (e.g., prostate cancer, leukemia, colorectal cancer, ovarian cancer, lung cancer, and testicular cancer), or cardiovascular disorder. A control level may be, for example, the level of the inhibition of the activity of the Runx2 protein by the AR protein in a normal subject.

A third method of the invention involves detecting the inhibition of the activity of the Runx1 protein by the AR protein in a subject. If the inhibition of the activity of the Runx1 protein by the AR protein in the subject is decreased compared to a control level, it indicates that the subject is likely to be suffering from or at risk for developing cancer (e.g., leukemia and prostate cancer), a bone disorder (e.g., osteoporosis), a disorder of the immune system (e.g., an autoimmune disorder), or a cardiovascular disorder. A control level may be, for example, the level of the inhibition of the activity of the Runx1 protein by the AR protein in a normal subject.

### Detection Methods

The invention provides methods for detecting Runx2-ERα, Runx2-AR, and Runx1-AR interactions. These methods may be used, e.g., in a screening method, a treatment method, or a diagnostic method.

More specifically, one detection method involves providing a system containing an ERα protein and a Runx2 protein including a PST domain, and detecting the interaction between the ERα protein and the PST domain of the Runx2 protein. The interaction between the ERα protein and the PST domain of the Runx2 protein can be detected by any of the methods known in the art, for example, GST pull-down and co-immunoprecipitation assays. The ERα protein may contain a DBD that interacts with the PST domain of the Runx2 protein.

Another detection method involves providing a system containing an AR protein including a DBD and a Runx2 protein including a Runt domain and a PST domain, and detecting the interaction between the DBD of the AR protein and the Runx2 protein, or the interaction between the AR protein and the Runt or PST domain of the Runx2 protein. The interaction between the DBD of the AR protein and the Runx2 protein and the interaction between the AR protein and the Runt or PST domain of the Runx2 protein can be detected by any of the methods known in the art, for example, GST pull-down and co-immunoprecipitation assays.

A third detection method involves providing a system containing an AR protein including an NTD and a LBD and a Runx1 protein, and detecting the interaction between the NTD or the LBD of the AR protein and the Runx1 protein. The interaction between the NTD or the LBD of the AR protein and the Runx1 protein can be detected by any of the methods known in the art, for example, GST pull-down and co-immunoprecipitation assays.

The methods of the invention are not limited to the Runx2-ERα, Runx2-AR, and Runx1-AR pairs. To the extent where applicable, these methods may be employed for any Runx protein (Runx1, 2, or 3) and a member of the nuclear receptor family such as progesterone receptor, peroxisome proliferator activated receptor (PPAR) α, β, or γ, retinoic acid receptor α, β, or γ, retinoid X receptor α, β, or γ, thyroid hormone receptor α or β, vitamin D3 receptor, mineralocorticoid receptor, or hepatocyte nuclear factor 4 (HNF4).

The following examples are intended to illustrate, but not to limit, the scope of the invention. While such examples are typical of those that might be used, other procedures known to those skilled in the art may alternatively be utilized. Indeed, those of ordinary skill in the art can readily envision and produce further embodiments, based on the teachings herein, without undue experimentation.

### EXAMPLES

### Example I

### Modulation of Runx2 Activity by Estrogen Receptor α: Implications for Osteoporosis and Breast Cancer

### Abstract

The transcription factors Runx2 and estrogen receptor α (ERα) are involved in numerous normal and disease processes, including postmenopausal osteoporosis and breast cancer. Using indirect immunofluorescence microscopy and pull down techniques we found them to co-localize and form complexes in an ligand-dependent manner. Estradiol-bound ERα strongly interacted with Runx2 directly through its DNA binding domains (DBD), and only indirectly through its N-terminal and ligand-binding domains. Runx2's amino acids 417-514, encompassing activation domain 3 and the nuclear matrix targeting sequence, were sufficient for interaction with ERα's DBD. As a consequence of the interaction, Runx2's transcriptional activation activity was strongly repressed, as shown by reporter assays in COS7 cells, breast cancer cells and late-stage MC3T3-E1 osteoblast cultures. Meta-analysis of gene expression in 779 breast cancer biopsies indicated negative correlation between the expression of ERα and Runx2 target genes. Selective ER modulators (SERMs) induced ERα-Runx2 interactions, but led to various functional outcomes. The regulation of Runx2 by ERα may play key roles in osteoblast and breast epithelial cell growth and differentiation; hence, modulation of Runx2 by native and synthetic ERα ligands offers new avenues in SERM evaluation and development.

### Introduction

The mammalian runt-related gene family encodes three transcription factors that play pivotal roles in lineage-specific cell growth and differentiation. Whereas Runx1 is implicated in definitive hematopoiesis and Runx3 (Runt-related transcription factor 3) in gut and nervous system development (1), Runx2 is a master transcription factor controlling osteoblast differentiation (2). Runx2-null mice lack osteoblasts and fail to form bone (3, 4). Low bone mass (LBM) has been observed in Runx2 heterozygous mice (5), mice missing one of two Runx2 isoforms (6), as well as transgenic mice whose osteoblasts express a dominant negative (DN) form of Runx2 under the control of the osteocalcin (OC) promoter (7). Runx2 has also been shown to act downstream of bone anabolic agents, including BMPs, PTH and estrogens (8-10). Surprisingly, however, transgenic mice whose osteoblasts over-express Runx2 under the control of the α1(I) collagen promoter also have LBM leading to spontaneous fractures (11, 12); and conversely, transgenic mice expressing a Runx2-DN under the control of the same promoter have high bone mass and are protected against ovariectomy-induced bone loss (9). Conceivably, therefore, pro-skeletal agents may inhibit Runx2 under some conditions in order to prevent bone loss.

The runt-related proteins also play important roles in cancer. Depending on the context, they can function as either tumor suppressors or promoters (13). Runx2 deficiency has been recently shown to promote immortalization and tumorigenesis (14). This is consistent with earlier studies of Runx1 and Runx3, in which inactivation of the former (via chromosomal translocation) and epigenetic silencing of the latter (via DNA methylation) were found associated with hematologic and gastric cancers, respectively (15). On the other hand, Runx2 has been implicated in cancer progression (13), including breast cancer bone metastasis (16). Possibly, transient inactivation of Runx2 promotes tumorigenesis, and its subsequent reactivation supports the metastatic phenotype.

Like Runx2, estrogens play critical roles in bone metabolism and carcinogenesis. Loss of estrogen function, at menopause or during anti-estrogen therapy for the management of hormone-driven malignancy, increases the risk for osteoporotic fractures, cardiovascular disease and dementia (17-19). On the other hand, estrogens have been associated with carcinogenesis (20), as hormone replacement therapy for postmenopausal women increases the risk for breast, endometrial and ovarian cancer (21). Molecular mechanisms of estrogens' carcinogenic activity are not well understood.

The pro-skeletal property of estrogens is multifaceted. Estrogens increase cell proliferation and survival in the osteoblast lineage, resulting in a bone anabolic effect (22-24). Most importantly, however, estrogens attenuate bone resorption and turnover, and this occurs via at least two mechanisms. First, estrogens directly promote osteoclast apoptosis (24), at least in part by stimulating FASL expression (25). Second, estrogens limit osteoclastogenesis from precursor cells, thereby attenuating bone turnover (26), a process that is skewed away from formation and towards resorption in most adults. Upon estrogen loss, increased osteoblast numbers fuel excessive bone turnover (23); and, in the absence of estrogens, each of these osteoblasts expresses higher levels of osteoclastogenic factors (24). Molecular mechanisms remain to be elucidated, by which estrogens attenuate osteoclastogenesis and osteoblast-driven osteoclastogenesis.

In our pursuit of interactions between sex steroids and Runx2, we made a novel observation, that estrogens strongly inhibit Runx2 in COS7 cells, breast cancer cells and late-stage MC3T3-E1 osteoblast cultures. We dissect this inhibitory activity in molecular terms, and discuss how it may contribute to two well-established activities of estrogens—the restraining of bone turnover and the promotion of breast carcinogenesis.

### Materials and Methods

### Materials

Flag antibodies were obtained from Sigma-Aldrich (St. Louis, Mo.). The other primary antibodies were from Santa Cruz Biotechnology (Santa Cruz, Calif.), including sc-10758 for Runx2 and sc-787 (or sc-7207 when indicated) for ERα. Fluorochrome-conjugated antibodies for confocal microscopy were obtained from Pierce Biotechnology (Rockford, Ill.). Estradiol, ICI 182780, and hydroxytamoxifen were from Sigma-Aldrich. Expression vectors encoding human ERα, ERβ, deletion constructs lacking the ligand binding domain (LBD) or the N-terminal domain (NTD), and ERα-GST-LBD, as well as ERE-luc were described previously (27-30). Human Runx1 and Runx2 expression vectors were obtained from Dr. Westendorf (Mayo Clinic, Rochester, Minn.). Mouse Runx2 expression vector and the 6XOSE2-luc Runx reporter were from Dr. Gerard Karsenty (Columbia University, New York, N.Y.). All other expression plasmids were prepared using standard cloning procedures and confirmed by sequencing.

### Cell Culture

COS7 cells were maintained in Dulbeco Modified Eagle Medium (DMEM) and 5% fetal bovine serum (FBS). The mouse osteoblastic cell line MC3T3-E1 was maintained in α-MEM and 10% FBS. Runx-reporter osteoblasts were generated by stable transfection of MC3T3-E1 cells with the 6XOSE2-luc reporter construct using the calcium phosphate coprecipitation method and hygromycin (100 ng/ml) as the selection drug. Resistance to hygromycin was conferred by cotransfection of the pCEP plasmid (1:15 molar ratio). To support development of the osteoblast phenotype, cells were treated with 50 μg/ml ascorbic acid and 10 nM β-glycerophosphate commencing at confluence. The human breast cancer cell lines MDA-MB-231, T47D, and MCF7 were maintained in DMEM and 5% FBS.

### Transient Transfections

Cells were seeded at a density of 10,000 per well in 96-well plates and grown in 5% charcoal-stripped serum (CSS)-containing phenol red-free medium for 24 h. The indicated plasmids (50 ng reporter, 52 ng total) were transfected using ‘LTX and Plus reagent’ according to manufacture's specification (Invitrogen, Carlsbad, Calif.). Equimolar amounts of the empty vectors pcDNA 3.1 and pSG5 were used as controls, and the pCAT-basic promoter-less plasmid was used as ‘filler’ DNA. Under these conditions the total amounts of promoter equivalents (molar) and DNA mass (ng) were the same in all wells of a particular experiment. As an internal control, 0.01 ng of tk-Renilla-luc or CMV-renilla-luc (Promega, Madison, Wis.) was used for correction of transfection efficiency. Cells were subsequently treated with vehicle (EtOH, 0.01%) or the indicated ligands for 24 h. Luciferase activity was determined using the dual-assay luciferase kit (Promega, Madison, Wis.).

### Co-Immunoprecipitation

Cells were seeded at a density of 300,000 per well in six-well plates and grown in 5% CSS-containing phenol red-free medium for 24 h. Cells were transfected when indicated, and 24 h later they were lysed in a 50 mM Tris-HCl buffer (pH 7.4) containing 150 mM NaCl, 1 mM EDTA, 1% Triton X-100, and fresh Protease Inhibitors Cocktail (1%; Sigma, St. Louis, Mo.). After homogenization by passing ten times through a 1 cc microfine insulin syringe, lysates were cleared by centrifugation at 14,000 rpm for 5 min in a bench-top microfuge, and 15% of the lysate solution was set aside and used as input. The remaining lysate was immunoprecipitated with approximately 3 μg of the specified antibody and 30 μl Protein-G beads (Amersham Biosciences Freiburg, Germany) and washed three times for 5 min each with the same buffer, followed by centrifugation at 4,000 rpm for 1 min. Thirty μl of the immunoprecipitate/bead suspension were mixed with 20 μl of 2.5× ‘sample buffer’ (50% Glycerol, 10% SDS, 1 M Tris-HCl, pH 6.8, 25% α-Mercaptoethanol, 0.5% Bromophenol blue) and boiled for 5 min. Input and immunoprecipitates were analyzed by Western blot analysis.

### GST Pull-Down Interaction Assay

Expression constructs for GST pull-down bait proteins were generated using pGEX-4T-1 (Amersham Biosciences, Freiburg, Germany) by in-frame fusions of DNA fragments encoding ERα's amino acids 1-180, 181-265, and 266-595, representing the NTD, DBD, and LBD respectively. Bait proteins were purified with the GST purification module (Amersham Biosciences) according to the manufacturer's protocol. The preys, ³⁵S-labeled full-length and fragments of Runx2, were prepared using TNT® T7 Quick Kit from Promega according to manufacturer's protocol. Ten μg of each bait, bound to glutathione-Sepharose, was incubated with the preys in 20 mM Tris-HCl (pH 7.5), 150 mM NaCl, 5 mM MgCl₂ and 0.01% Nonidet P-40, supplemented with Complete™ protease inhibitor mix (Roche Diagnostics). Bound proteins were analyzed by SDS-PAGE and autoradiography.

### Real Time RT-PCR

Total RNA was isolated using Aurum Total RNA kit (Bio-Rad, Hercules, Calif.) following the manufacturer's recommendations. One microgram of total RNA was reverse-transcribed (Invitrogen) and the cDNA was subjected to real-time PCR amplification (RT-qPCR) using IQ SYBR Green (Bio-Rad). PCR primers were designed using the Primer3 program (see the website frodo.wi.mit.edu/cgi-bin/primer3/primer3_www.cgi) and are listed in Table 1).

TABLE 1
Primer used for RT-qPCR

| | | |
|---|---|---|
| Gene | Forward Primer | Reverse Primer |

| | | |
|---|---|---|
| mOC | 5′-CGGCCCTGAGTCTGACAAA-3′ | 5′-GCCGGAGTCTGTTCACTACCTT-3′ |
| Luciferase | 5′-CCAGGGATTTCAGTCGATGT-3′ | 5-AATCTCACGCAGGCAGTTCT-3 |
| mRANKL | 5′-CGCTCTGTTCCTGTACCTTCG-3′ | 5-AGGCTTGTTTCATCCTG-3′ |
| mMMP9 | 5′-TACAGGGCCCCTTCCTTACT-3′ | 5′-CTGACGTGGGTTACCTCTGG-3′ |
| mL10A | 5′-CGCCGCAAGTTTCTGGAGAC-3′ | 5′-CTTGCCAGCCTTGTTTAGGC-3′ |

### Immunofluorescence

COS7 cells were grown on a 18 mm² coverslips in six well plates for 24 h using 5% CSS-containing phenol red-free DMEM. Cells were transfected with 100 ng each of ERα- and Runx2-expressing plasmids and treated with vehicle or 10 nM E2 for 24 h. Cells were then fixed with 95% methanol for 15 min and permeabilized with 1% saponin. ERα and Runx2 were visualized with the respective primary antibodies and secondary antibodies conjugated to either rhodamine or fluorescein, respectively. Cells were mounted using Vectashield Hard Set mounting medium with DAPI (Burlingame, Calif.). Cells were viewed using a LSM 510 Zeiss confocal microscope at 60× magnification. Images were processed using the default settings on Image J for colocalization finder, surface plot diagrams, and RG color picker. Using the default settings on the colocalization finder we quantitated the percentage colocalization where both red and green pixels overlapped.

### Microarray Data Mining

Normalized expression data was obtained from Oncomine (31). Runx2 target genes from the literature (Table 2) were subjected to an unsupervised 2-dimensional hierarchical cluster analysis using the software Jmp V 5.0 (see the website www.jmp.com). ERα target genes were mapped using Heatmap V 1.0 (see the website quertermous.stanford.edu/heatmap.htm) to the corresponding Runx2 cluster.

TABLE 2
Runx2 and ERα target gene subjected to meta-analysis

| | Gene | Accession # | References |
|---|---|---|---|
| Runx2 | ABCF1 | NM_001090 | (55, 58) |
| Targets | ACAT2 | NM_005891 | (55) |
| | AGC1 | NM_001135 | (55) |
| | ALPL | NM_000478 | (55) |
| | BGLAP | NM_199173 | (56) |
| | CCNB2 | NM_004701 | (57) |
| | CDC6 | NM_001254 | (57) |
| | CNN2 | NM_201277 | (58) |
| | Col9a1 | NM_001851 | (58) |
| | Col9A3 | NM_001853 | (58) |
| | DKK1 | NM_012242 | (55, 58) |
| | Dlx5 | NM_005221 | (55, 58) |
| | GTPbp2 | NM_019096 | (55) |
| | HCK | NM_002110 | (57) |
| | HLA-E | NM_005516 | (57) |
| | HSP105 | NM_006644 | (55) |
| | IBSP | NM_004967 | (55, 58) |
| | MATN4 | NM_003833 | (55, 58) |
| | MCM5 | NM_006739 | (57) |
| | MMP13 | NM_002427 | (55, 58) |
| | MMP9 | NM_004994 | (55, 58) |
| | MX1 | NM_002462 | (55) |
| | PCOLCE2 | NM_013363 | (55, 58) |
| | Pim1 | NM_002648 | (55) |
| | PLTP | NM_006227 | (55, 58) |
| | Rpl39l | NM_052969 | (55, 58) |
| | RSAD2 | NM_080657 | (55) |
| | SLC16A3 | NM_004207 | (55) |
| | Slc2a1 | NM_006516 | (55, 58) |
| | SOX9 | NM_000346 | (55, 58) |
| | SPP1 | NM_000582 | (55, 58) |
| | TAGLN2 | BC009357 | (55) |
| | TCF7 | NM_201632 | (55, 58) |
| | TGFBI | NM_000358 | (55, 57, 58) |
| | TRAM2 | NM_012288 | (58) |
| | TUBB4 | NM_006087 | (55) |
| | USP18 | NM_017414 | (55) |
| | VDR | NM_000376 | (55, 58) |
| | VEGFA | NM_003376 | (57) |
| | VIM | EF445046 | (55) |
| | WWP2 | NM_007014 | (55, 58) |
| ER and ER | ERα | NM_000125 | |
| Targets | PR | NM_000926 | (59) |
| | PS2 | NM_003225 | (59) |
| | GREB1 | NM_033090 | (59) |
| | NRIP1 | NM_003489 | (59) |

### Results

### ERα, not ERα, Inhibits Runx2 Activity in a Ligand-Specific Manner

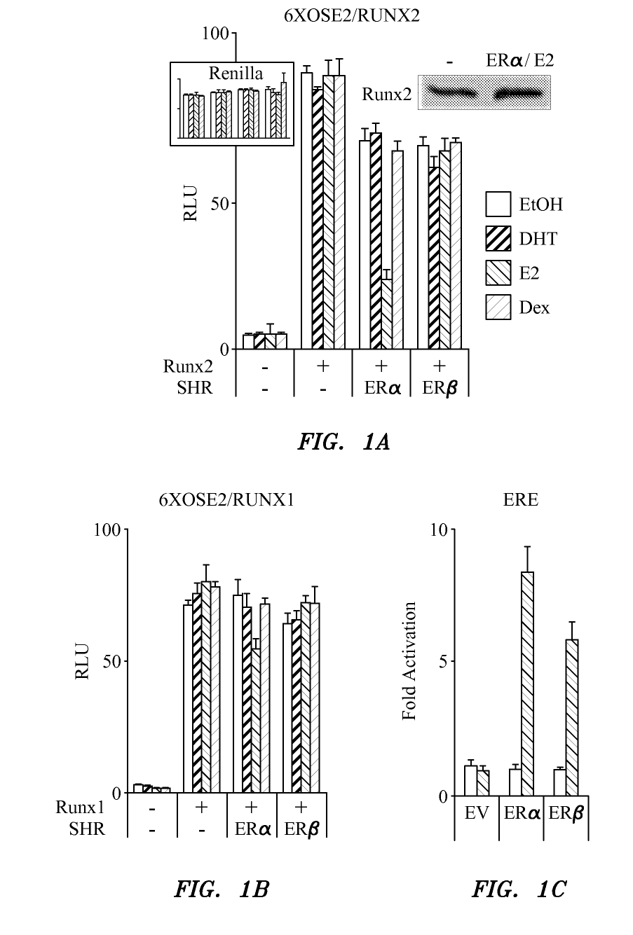

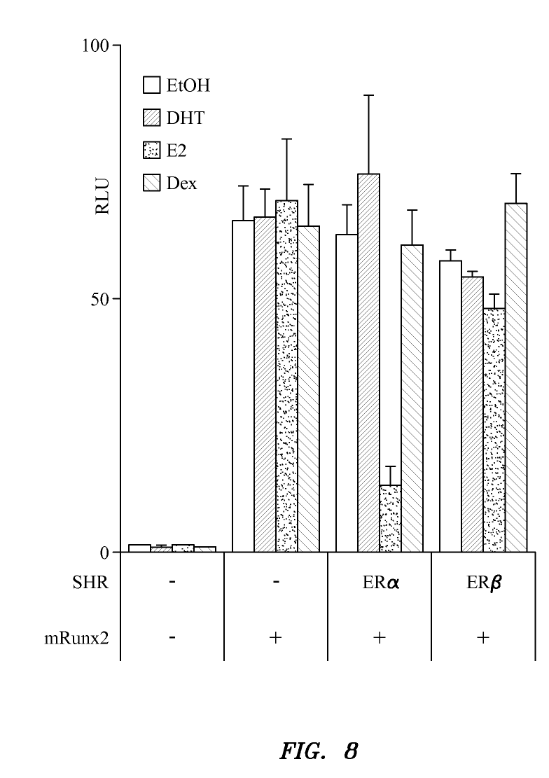

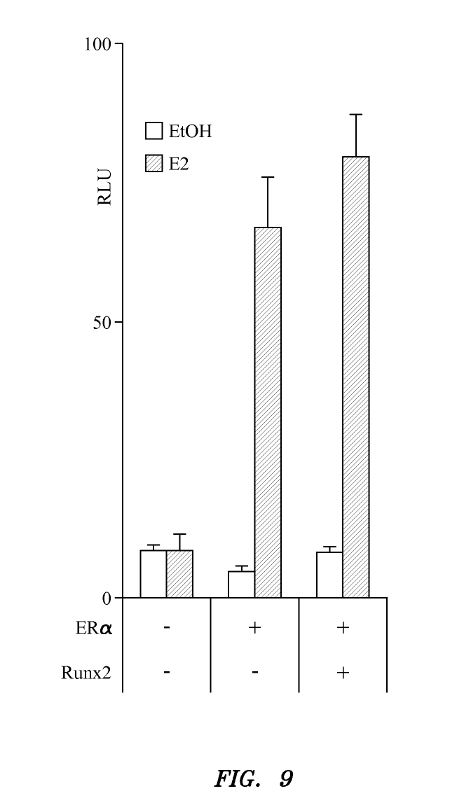

We initially investigated the influence of estradiol (E2) and its receptors ERα and ERβ on Runx2 after transfecting COS7 cells with the respective expression vectors along with the Runx2 reporter 6XOSE2-luc (32). As shown in FIG. 1A, E2 inhibited the activity of Runx2 by 76% in the presence of ERα. The inhibition ranged from 43% to 78% in eight independent experiments. E2 did not inhibit Runx2 activity in the presence of ERβ, although, as shown in FIG. 1C, the latter was almost as potent as ERα in stimulating a luciferase reporter driven by classical estrogen response elements (ERE). In the absence of ligand, both ERα and ERβ inhibited Runx2 only minimally (FIG. 1A). The strong inhibitory activity of ERα was elicited by E2, but not by dihydrotestosterone or dexamethasone (FIG. 1A). Similar to human Runx2 (FIG. 1), the mouse homolog was also inhibited by E2-bound ERα (FIG. 8). Interestingly, E2-activated ERα only slightly inhibited transcription from 6XOSE2-luc when the reporter was stimulated by Runx1 rather than Runx2 (FIG. 1B). These results indicate a strong and specific inhibitory mechanism, whereby E2-bound ERα suppresses Runx2's transactivation activity. In a complementary experiment, Runx2 did not affect ERα-mediated activation of an ERE-containing reporter (FIG. 9).

### Functional Mapping of ERα Domains Responsible for Runx2 Repression Indicates Independence of ERα's Transactivation Activity

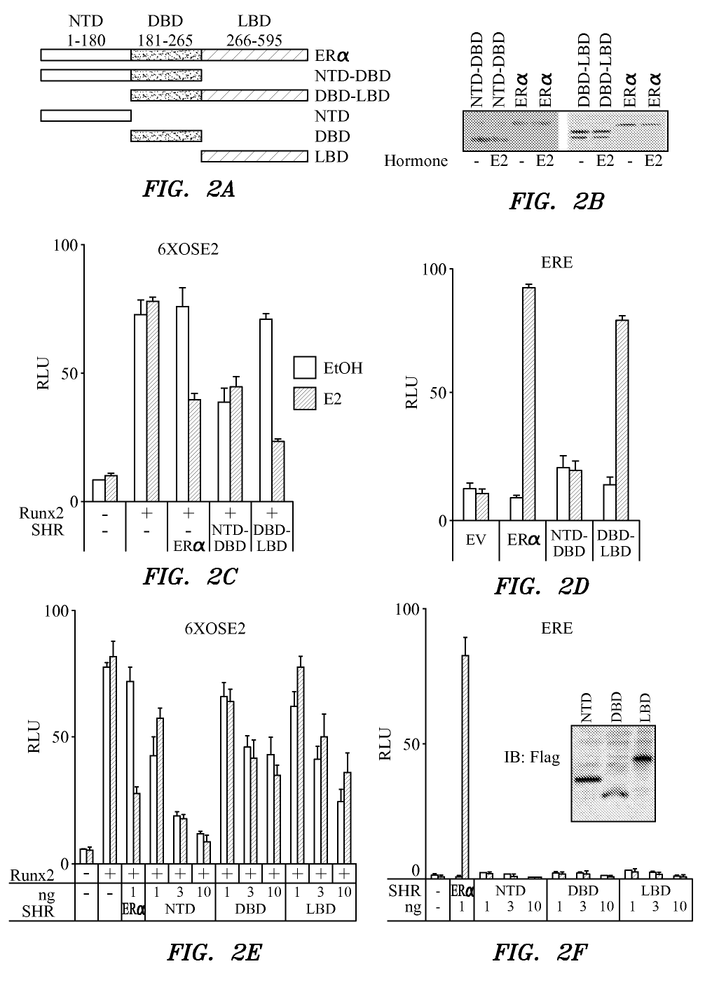

To functionally map the domain(s) of ERα responsible for Runx2 repression, and to test whether repression was dependent on ER-mediated stimulation of ERE-containing promoters, we measured the influence of the ERα deletion constructs illustrated in FIG. 2A on Runx2 activity. Stimulation of ERE-luc was assessed in parallel. Most importantly, deletion of the ERα-LBD, which was associated with near complete loss of ERE-luc activity in COS7 cells (FIG. 2D), resulted in constitutive full repression of Runx2 activity (FIG. 2C). Further suggesting dissociation between ERα's transcriptional activation activity from Runx2 repression, each of the ERα-NTD, -DBD and -LBD inhibited Runx2-mediated activation of 6XOSE-luc (FIG. 2E) while leaving ERE-luc (as well as CMV-renilla-luc and tk-renilla-luc) essentially unaffected (FIG. 2F), The ERαDBD-LBD also fully repressed Runx2 activity (FIG. 2C); and, just like full-length ERα, repression by the DBD-LBD was ligand dependent. These results suggest that ERα represses Runx2 without activating ERE-containing target genes, and that the repression mechanism employs multiple domains of ERα.

ERα Physically Interacts with Runx2 in a Ligand-Dependent Manner

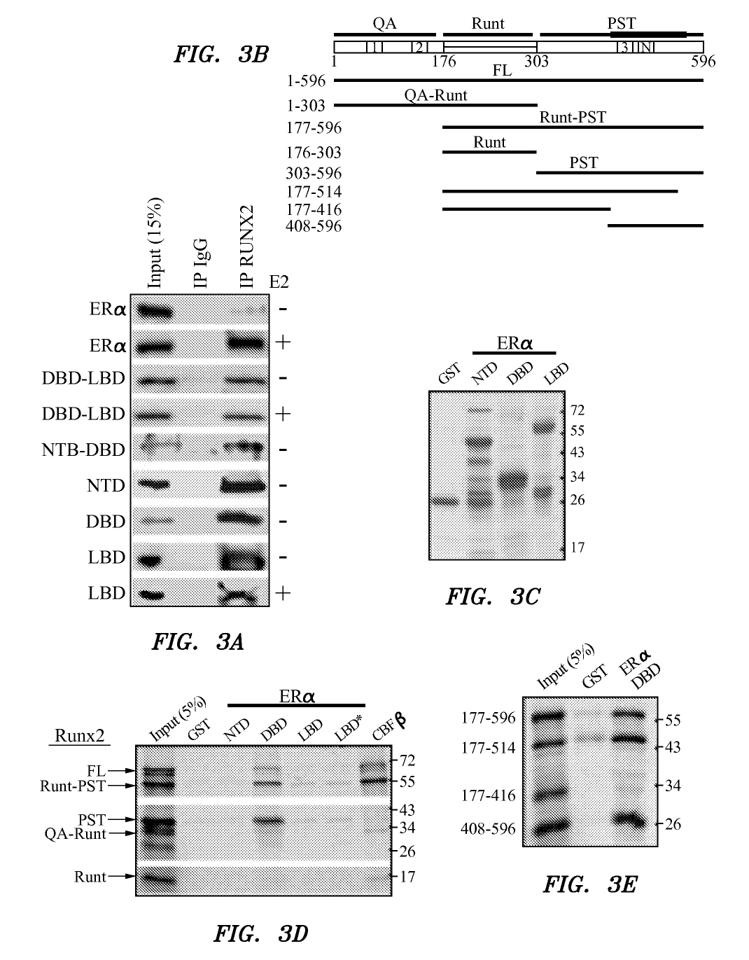

Because repression of Runx2 is independent of ERα's transactivation activity, it could occur via physical interaction between the two proteins. Indeed, ERα was present in Runx2 immunoprecipitates; the ERα/Runx2 interaction was weak in untreated cells, and increased dramatically upon E2 treatment (FIG. 3A). A series of ERα deletion constructs, including the DBD-LBD, NTD-DBD, as well as the individual domains, were also employed in the co-immunoprecipitation (co-IP) assay, and each of them was associated with Runx2 (FIG. 3A). However, unlike the full-length receptor, the receptor fragments tested did not require E2 to form complexes with Runx2 (FIG. 3A). These data suggest that ERα interacts with Runx2 through multiple surfaces, and that these surfaces are exposed only after the full-length protein has assumed a permissive ligand-induced conformation.

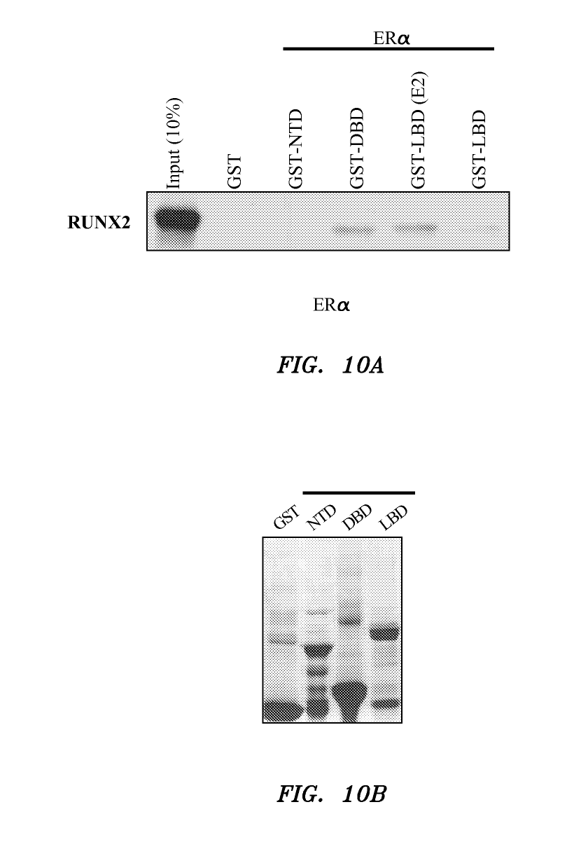

To test if the physical interactions demonstrated by co-IP are direct, we asked whether immobilized ERα domains (FIG. 3C) could pull down in vitro transcribed and translated Runx2. In these GST pull-down assays, the ERα-DBD strongly interacted with Runx2, and this strong interaction was recapitulated with Runx2's PST domain (alone or with the Runt domain), but not with the QA-Runt or Runt domains (FIG. 3D). The PST domain continued to interact with ERα's DBD after deletion of the 515-596, but not the 417-596 amino acid sequence (FIG. 3D). Positive pull-down was also observed after deletion of the PST's N-terminal 303-407 amino acids sequence, thereby mapping the interaction surface to Runx2's amino acids 417-514 (FIG. 3E), which contain activation domain 3 (AD3) and the nuclear matrix targeting sequences (NMTS). In contrast to ERα's DBD, the NTD did not interact with full-length or fragments of Runx2 (FIG. 3D). The ERα-LBD displayed a weak interaction with Runx2, which, like the ERα-DBD, mapped to Runx2's PST domain (FIG. 3D). This interaction was not influenced by physiological estrogen concentrations (FIG. 3D), but was increased in the presence of 1 μM E2 (FIG. 10). Thus, ligand-dependent ERα/Runx2 interaction is mediated primarily by a contact between Runx2's amino acids 417-514 within the PST and ERα's-DBD, a potential secondary direct contact of Runx2 with the ERα-LBD (via the PST domain), and an indirect contact with the NTD.

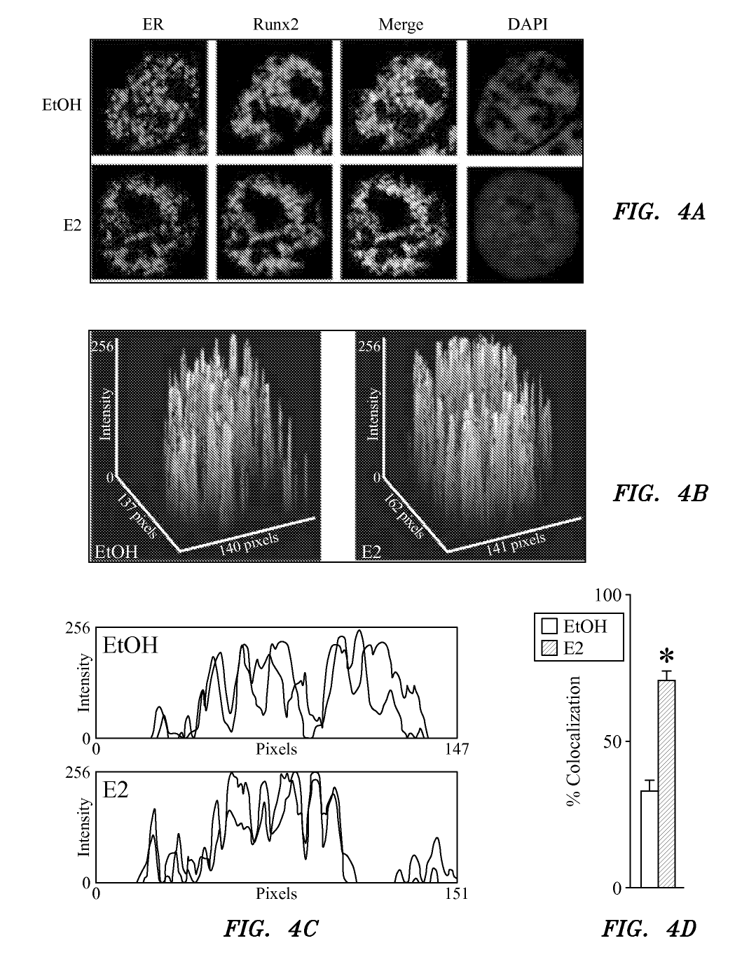

Indirect confocal fluorescence microscopy was then performed to test whether ERα co-localized with Runx2 in living cells (FIG. 4). Both transcription factors were concentrated in the nucleus regardless of E2 treatment, and occupied distinct domains. Quantitative analysis of 10 randomly selected untreated cells indicated 34% co-localization (FIG. 4D), where ERα and Runx2 could interact in vivo. Upon treatment with E2, there was a decrease in areas occupied by either protein alone, with a concomitant increase in areas containing both proteins together (FIGS. 4A-C). Quantitative analysis of 10 randomly selected E2-treated cells indicated a highly significant (p<8.3×10⁻⁸) 2.1-fold increase in co-localization compared to the 10 randomly selected untreated cells (FIG. 4D), likely reflecting the E2-induced physical interaction between the two transcription factors (see FIG. 3).

### Inhibition of Runx2 by Estradiol in Late Stage MC3T3-E1 Osteoblast Cultures

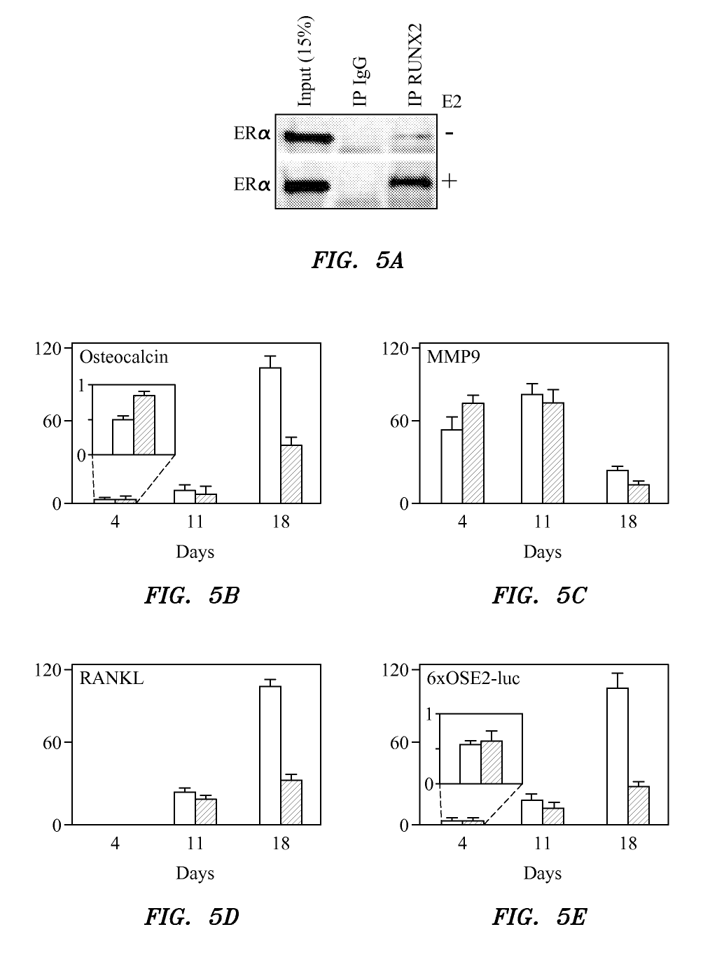

To address the ERα-Runx2 interaction in osteoblasts, we first confirmed by co-IP assay the physical association between endogenous ERα and endogenous Runx2 in MC3T3-E1 osteoblasts (FIG. 5A). The influence of E2 on Runx2 was then examined during the development of the osteoblast phenotype in cultures of MC3T3-E1 cells that had been stably transfected with the 6XOSE2-luc Runx2 reporter construct (32). We measured the expression of the mRNAs for OC, a classical Runx2 target (33, 34); MMP9, also a Runx2 target (35); RANKL, which may be regulated by Runx2 (36); and luciferase (driven by the six Runx2 binding sites). Whereas OC and MMP9 were at best weakly stimulated on day 4, all mRNAs were suppressed on day 18 (FIGS. 5B-E). Thus, while in early cultures Runx2 may be stimulated by E2, the results demonstrate E2-mediated repression in late MC3T3-E1 cultures, which is similar to the inhibition observed in COS7 cells (FIG. 1) and breast cancer cells (see below). Although E2 did not affect mineralization in the MC3T3-E1 cultures, inhibition of RANKL and MMP9 may attenuate their osteoclastogenic activity.

### Synthetic ER Ligands Inhibit or Stimulate Runx2 in a Compound- and Cell Type-Specific Manner

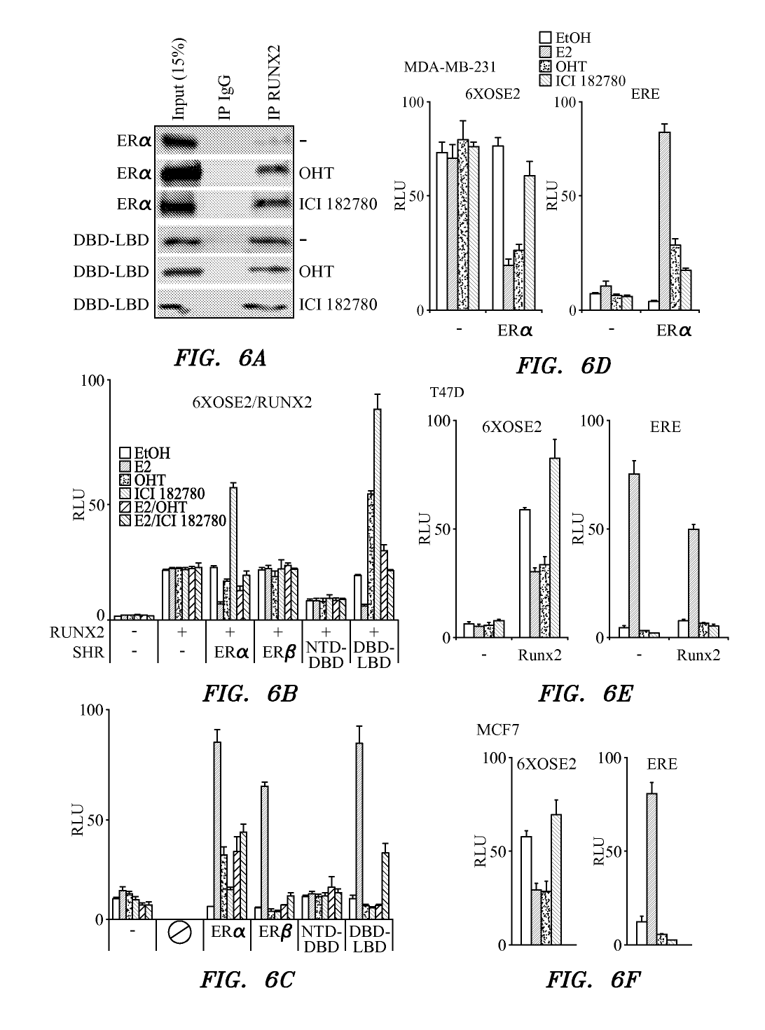

Selective estrogen receptor modulators (SERMs) are widely used for the management of breast cancer (37). A variety of SERMs are available, with different levels of partial agonism or antagonism with respect to the native ligand. Different SERMs induce in the receptor a variety of conformational changes, reflected for example, by increased sensitivity of helix 12 to trypsin digestion in the presence of ICI compounds as compared to E2, and even higher sensitivity in the presence of OHT (38). We first asked whether these SERMs mimicked E2 in inducing interaction with Runx2. As demonstrated by co-IP assays, each of OHT and ICI 182780 strongly induced the formation of ERα/Runx2 complexes (FIG. 6A). Remarkably, however, the functional consequences with regard to Runx2 activity were different compared to E2. While OHT slightly inhibited Runx2 activity, ICI 182780 had the opposite effect, stimulating Runx2 activity by 3.1-fold (FIG. 6B). This contrasted with the activity of these SERMs on an ERE-containing template, where ICI 182780 and OHT had weak and stronger partial agonist effects, respectively (FIG. 6C). Interestingly, OHT also had a stimulatory effect on Runx2 when bound to the ERαDBD-LBD (FIG. 6B), raising the possibility that different domains of OHT-bound ERα have opposing effects on Runx2.

We subsequently examined the effects of SERMs on Runx2 in breast cancer cells. When ERα was expressed in the ER-negative MDA-MB-231 cell line, E2 strongly inhibited the co-transfected 6XOSE2 Runx2 reporter (FIG. 6D). Unlike COS7 cells, OHT inhibited Runx2 in MDA-MB-231 cells as strongly as E2 (FIG. 6D). ICI 182780, which stimulated Runx2 in COS7 cells (FIG. 6B), had a slight if any inhibitory effect on Runx2 in MDA-MB-231 cells (FIG. 6D). In T47D and MCF7 beast cancer cells, ICI 182780 slightly stimulated Runx2 (FIGS. 6E-F), whereas the effect of OHT was uniformly inhibitory across all three breast cancer cell lines (FIGS. 6D-F). Thus, OHT mimics the strong E2-mediated inhibition of Runx2 specifically in breast cancer cells; and ICI 182780, which stimulates Runx2 in COS7 cells, only mildly regulates Runx2 activity in breast cancer cells, either upwards or downwards. The various effects of SERM-bound ERα on Runx2 activity may be relevant to their selective effects in different tissues in vivo.

### Negative Correlation Between ERα and Runx2 Target Genes in Breast Cancer Tissues

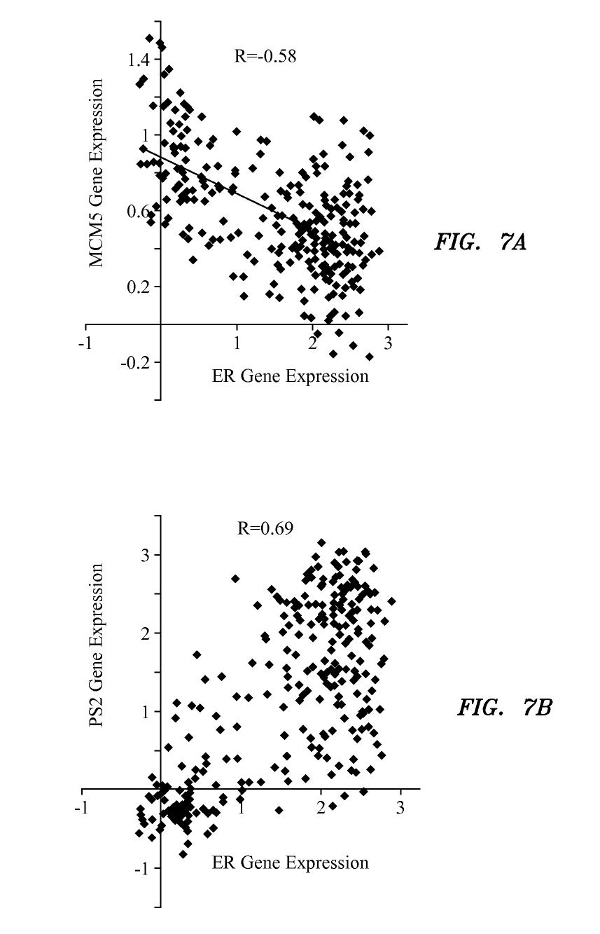

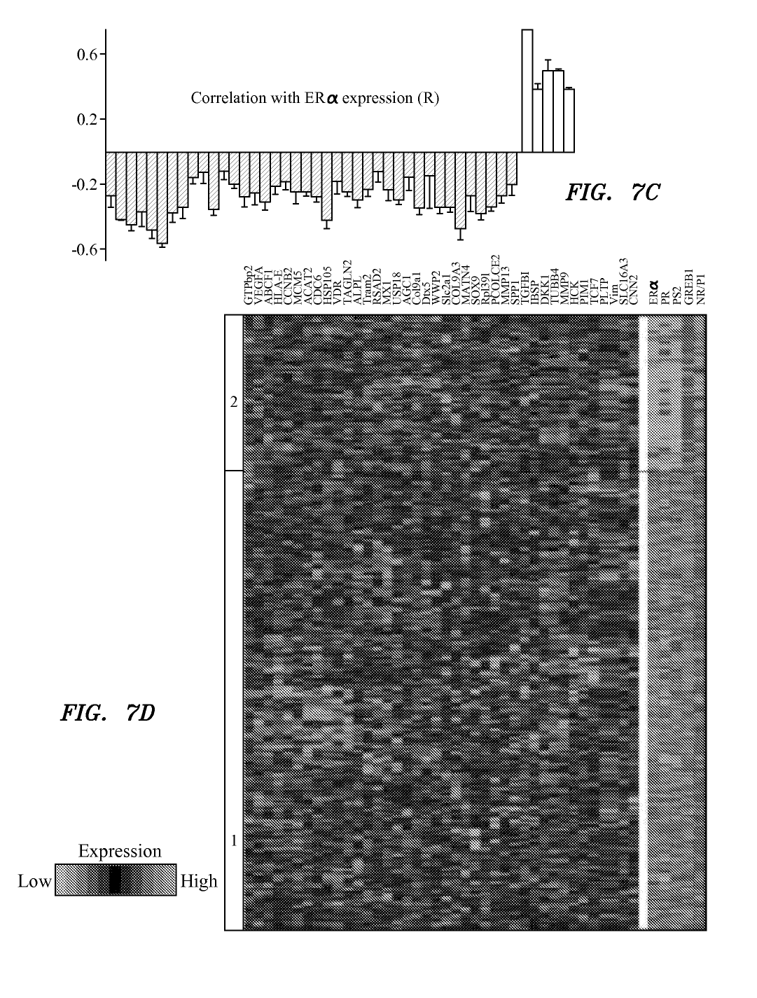

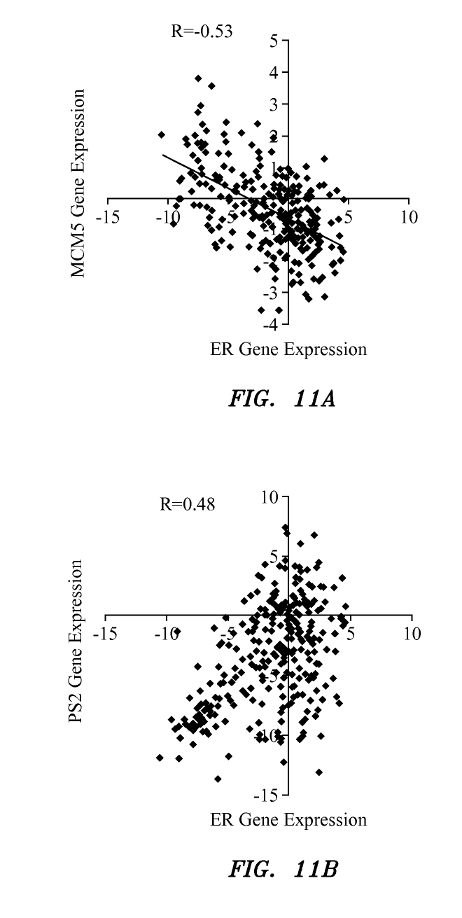

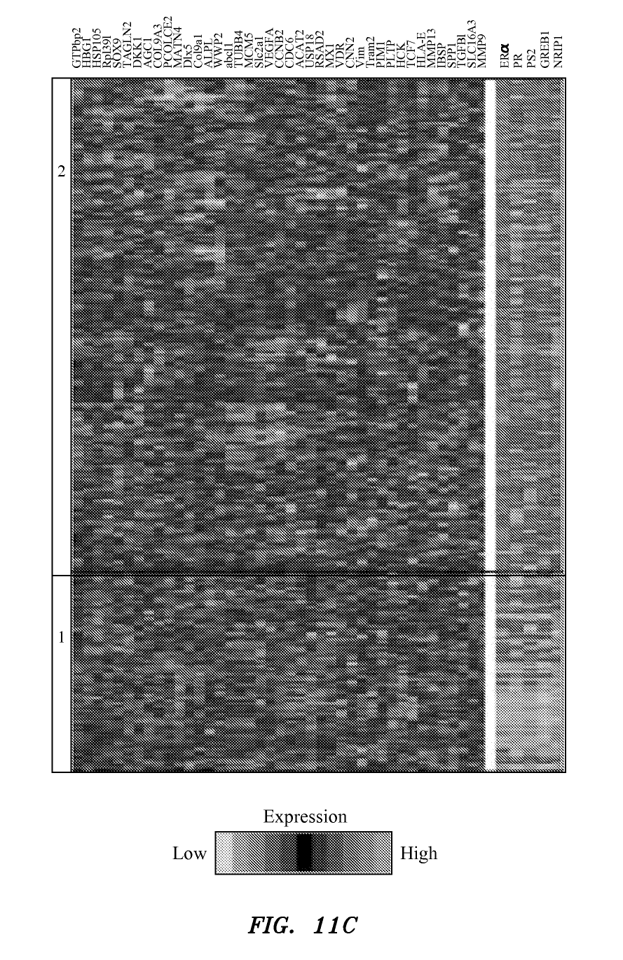

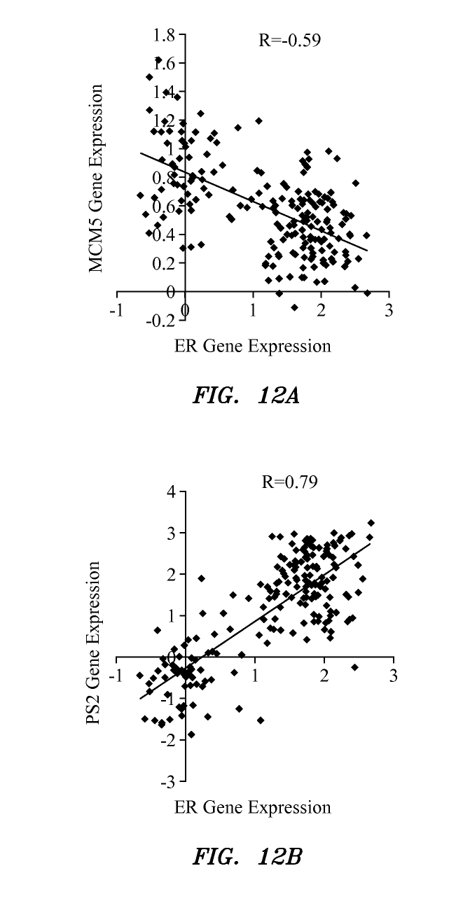

If ERα inhibits Runx2 in breast cancer, then one would expect to see an inverse relationship between the expression of ERα and Runx2 target genes in breast cancer biopsies. We therefore mined three datasets from Oncomine (31) for the expression of 41 known Runx2 target genes. The datasets represented comprehensive gene expression analyses of three cohorts of 286, 295, and 198 breast cancer biopsies (39-41). The 41 Runx2 target genes, listed in Table 2, were the top hits from each of three unbiased studies designed to discover Runx2 targets (42-44). Remarkably, expression of each of the Runx2 target genes tested was negatively correlated with the expression of ERα across each of the three cohorts. For example, in the series of 286 biopsies, the correlation coefficient between ERα expression and that of the Runx2 target gene MCM5 (43) was −0.58 (FIG. 7A). This correlation was close to the positive correlation between the expression of ERα and its classical target pS2 (R=0.69; FIG. 7B). Similar observations were made in the other two series (FIGS. 11A,B and 12A,B, respectively). Meta-analysis of the three studies altogether revealed a statistically significant negative correlation between ERα expression and each of the 41 Runx2 target genes (FIG. 7C). The average correlation coefficient was −0.29, with a standard error of 0.05 (n=41), compared with 0.59±0.05 (n=4) for classical ERα targets (FIG. 70). A significant negative correlation (p=0.03) was also observed between the expression of ERα and OC. To visually demonstrate the correlation between the expression of ERα and Runx2 target genes, we subjected the 41 Runx2 target genes to unsupervised clustering and examined the expression of ERα and ERα target genes in each cluster. In each of the three studies, we observed clusters that indicated inverse relationship between genes regulated by Runx2 versus ERα and its targets. For example, the cohort of 286 biopsies was clustered into two main branches, one with low expression of Runx2 target genes and high expression of ERα and its targets (FIG. 7D, cluster 1) and the other displaying a mirror image (FIG. 7D, cluster 2). Similar relationships were observed with the other two cohorts (FIGS. 11C and 12C). The negative correlation between expression of ERα and Runx2 target genes is consistent with the idea that inhibition of Runx2 by ERα occurs not only in breast cancer cell lines (FIG. 6) but also in human breast epithelial cells in vivo.

### Discussion

Two ERs, α and β, mediate the various actions of estrogens. In addition to their classical action on ERE-containing promoters, the ligand-activated receptors form complexes with, and modulate the activity of other regulatory proteins, including signal transducers (45) and transcription factors. For example, estrogens have been shown to activate AP-1 and SP1, resulting in increased expression of cyclin D1 (46). Here we show that ERα interacts with and inhibits Runx2 in osteoblasts and breast cancer cells. Transfection experiments comparing ERα and ERβ in COS7 cells show that the inhibition is specific for ERα, the same receptor that also mediates the physiological effects of estrogens in bone (47, 48) and breast (49).

E2 was required for ERα/Runx2 interaction in both the co-IP and the functional inhibition assays. Ligand is also required for ER interaction with transcriptional co-activators, which results in stimulation of ERE-containing promoters. However, Runx2 repression by ERα does not require classical activation through EREs, because (i) the NTD-DBD, as well as individual domains of ERα each suppressed Runx2 activity without activating EREs; (ii) the transcriptionally-competent ERα did not inhibit Runx2; and (iii) the partial agonist activity of ICI 182780 with regard to classical ERE activation was associated with stimulation, rather than inhibition of Runx2. Furthermore, dose-response curves constructed in parallel for the two activities of ERα showed that E2 represses Runx2-mediated activation of 6XOSE2-luc at concentrations lower than those necessary for activation of ERE-luc. Thus, Runx2 repression is dissociable from ERα's canonical transcriptional activation activity, and appears to occur via direct protein-protein interaction. Among possible mechanisms leading to inhibition of Runx2 by ERα, the two proteins could be present together at OSE2-like sites and form platforms for the further recruitment of co-repressors or co-activators, depending on the ERα ligand.

According to our co-IP data, each of ERα's NTD, DBD and LBD interacts with Runx2, although strong direct interaction was observed in the GST pull-own assay only with the DBD. These results are consistent with those of McCarthy et al., who used a two-hybrid assay to demonstrate that each of the ERα domains, and mostly the DBD, interacted with Runx2 (10). In this study, however, the reciprocal two-hybrid analysis mapped the interacting surfaces in Runx2 to both the QA and the PST domains, whereas our GST pull-down assay indicated interaction only at the PST domain. Be that as it may, the fine mapping of the interaction surface to Runx2's amino acids 417-514 within the PST domain raises the interesting possibility that ERα inhibits Runx2 by masking its C-terminal activation domain and/or its nuclear matrix targeting sequence (2).

The suggested inhibition of Runx2 by ERα in breast cancer cells in vivo could be either pro- or anti-oncogenic depending on the presiding function of Runx2. Initially, Runx2 likely plays a tumor suppressive role, and thus the E2-mediated inhibition at this stage would constitute a mechanism of hormonal carcinogenesis. However, Runx2 activity in later stages of cancer progression may promote expression of the metastatic phenotype (16). Therefore, in advanced breast cancer, inhibition of Runx2 by ERα may become beneficial. This could possibly explain why, despite the well-established oncogenic activity of ER signaling in breast epithelial cells (50), breast cancers that maintain ERα expression during tumor progression are generally less aggressive than ERα-negative tumors; in these tumors Runx2 would promote metastasis without ERα opposition.

In osteoblasts, estrogens influence Runx2 in a developmental stage-specific manner. Inhibition of Runx2 and its target genes OC and MMP9 is observed in late MC3T3-E1 cultures, when Runx2 activity and OC expression are maximal. However, data presented here and elsewhere (10, 51) show that in early MC3T3-E1 and in primary osteoblast cultures, estrogens do not inhibit, and may even stimulate Runx2 activity and OC expression. Both the stimulation and inhibition of Runx2 by estrogens could contribute to their pro-skeletal properties. During specific differentiation stages, stimulation of Runx2 may promote osteoblast function and bone formation. During other stages, inhibition of Runx2 may benefit the skeleton by (i) attenuating osteoclastogenesis thereby restraining bone turnover (23, 26, 52); and (ii) decreasing the expression of osteoclastogenic genes, possibly RANKL and MMP9, which were down-regulated along with OC in our late MC3T3-E1 cultures. The skeletal benefits of keeping Runx2 in check is suggested by the excessive endosteal bone resorption (reminiscent of postmenopausal bone loss) and spontaneous fractures observed in transgenic mice over-expressing Runx2 (11, 12), as well as the resistance of Runx2-DN transgenic mice to ovariectomy-induced bone loss (9).

Inhibition of Runx2 is a potential novel mechanism of action of estrogen in breast and bone, and may contribute to drug discovery. Specifically, if the inhibition were indeed important for bone and breast epithelial cell growth and differentiation, then SERMs would behave most “naturally” if they mimicked estrogens not only in stimulating ERE-driven transcription, but also in restraining Runx2. Furthermore, indications for SERM therapy for breast cancer should possibly take into account the potential consequences on Runx2 activity.

In conclusion, we documented and dissected strong physical interaction and inhibition of Runx2 by ERα. Suppression of Runx2 in response to E2, and the various responses to synthetic ligands, may mediate some of their effects on bone, breast and other organ systems. In addition, Runx2 inhibition assays may prove useful in SERM evaluation and development.

### REFERENCES

1. Coffman J A 2003 Runx transcription factors and the developmental balance between cell proliferation and differentiation. Cell Biol Int 27:315-324
2. Schroeder T M, Jensen E D, Westendorf J J 2005 Runx2: a master organizer of gene transcription in developing and maturing osteoblasts. Birth Defects Res C Embryo Today 75:213-225
3. Komori T, Yagi H, Nomura S, Yamaguchi A, Sasaki K, Deguchi K, Shimizu Y, Bronson R T, Gao M I, Inada M, Sato M, Okamoto R, Kitamura Y, Yoshiki S, Kishimoto T 1997 Targeted disruption of Cbfa1 results in a complete lack of bone formation owing to maturational arrest of osteoblasts [see comments]. Cell 89:755-764
4. Otto F, Thornell A P, Crompton T, Denzel A, Gilmour K C, Rosewel I R, Stamp G W, Beddington R S, Mundlos S, Olsen B R, Selby P B, Owen M J 1997 Cbfa1, a candidate gene for cleidocranial dysplasia syndrome, is essential for osteoblast differentiation and bone development [see comments]. Cell 89:765-771
5. Salingcarnboriboon R, Tsuji K, Komori T, Nakashima K, Ezura Y, Noda M 2006 Runx2 is a target of mechanical unloading to alter osteoblastic activity and bone formation in vivo. Endocrinology 147:2296-2305
6. Xiao Z, Awad H A, Liu S, Mahlios J, Zhang S, Guilak F, Mayo M S, Quarles L D 2005 Selective Runx2-II deficiency leads to low-turnover osteopenia in adult mice. Dev Biol 283:345-356
7. Ducy P, Starbuck M, Priemel M, Shen J, Pinero G, Geofiroy V, Amling M, Karsenty G 1999 A Cbfa1-dependent genetic pathway controls bone formation beyond embryonic development. Genes & development 13:1025-1036
8. Lee M H, Kim Y J, Kim H J, Park H D, Kang A R, Kyung H M, Sung J H, Wozney J M, Kim H J, Ryoo H M 2003 BMP-2-induced Runx2 expression is mediated by Dlx5, and TGF-beta 1 opposes the BMP-2-induced osteoblast differentiation by suppression of Dlx5 expression. The Journal of biological chemistry 278:34387-34394
9. Maruyama Z, Yoshida C A, Furuichi T, Amizuka N, Ito M, Fukuyama R, Miyazaki T, Kitaura H, Nakamura K, Fujita T, Kanatani N, Moriishi T, Yamana K, Liu W, Kawaguchi H, Nakamura K, Komori T 2007 Runx2 determines bone maturity and turnover rate in postnatal bone development and is involved in bone loss in estrogen deficiency. Dev Dyn 236:1876-1890
10. McCarthy T L, Chang W Z, Liu Y, Centrella M 2003 Runx2 integrates estrogen activity in osteoblasts. The Journal of biological chemistry 278:43121-43129
11. Liu W, Toyosawa S, Furuichi T, Kanatani N, Yoshida C, Liu Y, Himeno M, Narai S, Yamaguchi A, Komori T 2001 Overexpression of Cbfa1 in osteoblasts inhibits osteoblast maturation and causes osteopenia with multiple fractures. J Cell Biol 155:157-166
12. Geoffroy V, Kneissel M, Fournier B, Boyde A, Matthias P 2002 High bone resorption in adult aging transgenic mice overexpressing cbfa1/runx2 in cells of the osteoblastic lineage. Mol Cell Biol 22:6222-6233
13. Blyth K, Cameron E R, Neil J C 2005 The RUNX genes: gain or loss of function in cancer. Nature reviews 5:376-387
14. Zaidi S K, Pande S, Pratap J, Gaur T, Grigoriu S, Ali S A, Stein J L, Lian J B, van Wijnen A J, Stein G S 2007 Runx2 deficiency and defective subnuclear targeting bypass senescence to promote immortalization and tumorigenic potential. Proceedings of the National Academy of Sciences of the United States of America 104:19861-19866
15. Ito Y 2004 Oncogenic potential of the RUNX gene family: ‘overview’. Oncogene 23:4198-4208
16. Barnes G L, Hebert K E, Kamal M, Javed A, Einhorn T A, Lian J B, Stein G S, Gerstenfeld L C 2004 Fidelity of Runx2 activity in breast cancer cells is required for the generation of metastases-associated osteolytic disease. Cancer Res 64:4506-4513
17. Lindsay R 1991 Estrogens, bone mass, and osteoporotic fracture. Am J Med 91:10 S-13S
18. Mendelsohn M E, Karas R H 1999 The protective effects of estrogen on the cardiovascular system. N Engl J Med 340:1801-1811
19. Yaffe K 2001 Estrogens, selective estrogen receptor modulators, and dementia: what is the evidence? Ann N Y Acad Sci 949:215-222
20. Platet N, Cathiard A M, Gleizes M, Garcia M 2004 Estrogens and their receptors in breast cancer progression: a dual role in cancer proliferation and invasion. Critical reviews in oncology/hematology 51:55-67
21. Persson I 2000 Estrogens in the causation of breast, endometrial and ovarian cancers—evidence and hypotheses from epidemiological findings. J Steroid Biochem Mol Biol 74:357-364
22. Riggs B L, Yhosla S, Melton L J, 3rd 2002 Sex steroids and the construction and conservation of the adult skeleton. Endocr Rev 23:279-302
23. Manolagas S C, Kousteni S, Jilka R L 2002 Sex steroids and bone. Recent Prog Horm Res 57:385-409
24. Syed F, Khosla S 2005 Mechanisms of sex steroid effects on bone. Biochem Biophys Res Commun 328:688-696
25. Nakamura T, Imai Y, Matsumoto T, Sato S, Takeuchi K, Igarashi K, Harada Y, Azuma Y, Krust A, Yamamoto Y, Nishina H, Takeda S, Takayanagi H, Metzger D, Kanno J, Takaoka K, Martin T J, Chambon P, Kato S 2007 Estrogen Prevents Bone Loss via Estrogen Receptor alpha and Induction of Fas Ligand in Osteoclasts. Cell 130:811-823
26. Di Gregorio G B, Yamamoto M, Ali A A, Abe E, Roberson P, Manolagas S C, Jilka R L 2001 Attenuation of the self-renewal of transit-amplifying osteoblast progenitors in the murine bone marrow by 17 beta-estradiol. J Clin Invest 107:803-812
27. Ding X F, Anderson C M, Ma H, Hong H, Uht R M, Kushner P J, Stallcup M R 1998 Nuclear receptor-binding sites of coactivators glucocorticoid receptor interacting protein 1 (GRIP1) and steroid receptor coactivator 1 (SRC-1): multiple motifs with different binding specificities. Mol Endocrinol 12:302-313
28. Webb P, Nguyen P, Shinsako J, Anderson C, Feng W, Nguyen M P, Chen D, Huang S M, Subramanian S, McKinerney E, Katzenellenbogen B S, Stallcup M R, Kushner P J 1998 Estrogen receptor activation function 1 works by binding p160 coactivator proteins. Mol Endocrinol 12:1605-1618
29. Norris J D, Fan D, Stallcup M R, McDonnell D P 1998 Enhancement of estrogen receptor transcriptional activity by the coactivator GRIP-1 highlights the role of activation function 2 in determining estrogen receptor pharmacology. Biol Chem 273:6679-6688
30. Kim J H, Yang C K, Stallcup M R 2006 Downstream signaling mechanism of the C-terminal activation domain of transcriptional coactivator CoCoA. Nucleic Acids Res 34:2736-2750
31. Rhodes D R, Kalyana-Sundaram S, Mahavisno V, Varambally R, Yu J, Briggs B B, Barrette T R, Anstet M J, Kincead-Beal C, Kulkarni P, Varambally S, Ghosh D, Chinnaiyan A M 2007 Oncomine 3.0: genes, pathways, and networks in a collection of 18,000 cancer gene expression profiles. Neoplasia 9:166-180
32. Ducy P, Karsenty G 1995 Two distinct osteoblast-specific cis-acting elements control expression of a mouse osteocalcin gene. Mol Cell Biol 15:1858-1869
33. Ducy P, Zhang R, Geoffroy V, Ridall A L, Karsenty G 1997 Os2/Cbfa1: a transcriptional activator of osteoblast differentiation. Cell 89:747-754
34. Banerjee C, McCabe L R, Choi J Y, Hiebert S W, Stein J L, Stein G S, Lian J B 1997 Runt homology domain proteins in osteoblast differentiation: AML3/CBFA1 is a major component of a bone-specific complex. J Cell Biochem 66:1-8
35. Pratap J, Javed A, Languino L R, van Wijnen A J, Stein J L, Stein G S, Lian J B 2005 The Runx2 osteogenic transcription factor regulates matrix metalloproteinase 9 in bone metastatic cancer cells and controls cell invasion. Mol Cell Biol 25:8581-8591
36. Fu Q, Manolagas S C, O'Brien C A 2006 Parathyroid hormone controls receptor activator of NF-kappaB ligand gene expression via a distant transcriptional enhancer. Mol Cell Biol 26:6453-6468
37. Jordan V C, O'Malley B W 2007 Selective estrogen-receptor modulators and antihormonal resistance in breast cancer. J Clin Oncol 25:5815-5824
38. Tamrazi A, Carlson K E, Katzenellenbogen J A 2003 Molecular sensors of estrogen receptor conformations and dynamics. Mol Endocrinol 17:2593-2602
39. Wang Y, Klijn J G, Zhang Y, Sieuwerts A M, Look M P, Yang F, Talantov D, Timmermans M, Meijer-van Gelder M E, Yu J, Jatkoe T, Berns E M, Atkins D, Foekens J A 2005 Gene-expression profiles to predict distant metastasis of lymph-node-negative primary breast cancer. Lancet 365:671-679
40. Desmedt C, Sotiriou C 2006 Proliferation: the most prominent predictor of clinical outcome in breast cancer. Cell Cycle 5:2198-2202
41. van de Vijver M J, He Y D, van't Veer L J, Dai H, Hart A A, Voskuil D W, Schreiber G J, Peterse J L, Roberts C, Marton M J, Parrish M, Atsma D, Witteveen A, Glas A, Delahaye L, van der Velde T, Bartelink H, Rodenhuis S, Rutgers E T, Friend S H, Bernards R 2002 A gene-expression signature as a predictor of survival in breast cancer. N Engl J Med 347:1999-2009
42. Hecht J, Seitz V, Urban M, Wagner F, Robinson P N, Stiege A, Dieterich C, Kornak U, Wilkening U, Brieske N, Zwingman C, Kidess A, Stricker S, Mundlos S 2007 Detection of novel skeletogenesis target genes by comprehensive analysis of a Runx2(−/−) mouse model. Gene Expr Patterns 7:102-112
43. Young D W, Hassan M Q, Yang X Q, Galindo M, Javed A, Zaidi S K, Furcinitti P, Lapointe D, Montecino M, Lian J B, Stein J L, van Wijnen A J, Stein G S 2007 Mitotic retention of gene expression patterns by the cell fate-determining transcription factor Runx2. Proceedings of the National Academy of Sciences of the United States of America 104:3189-3194
44. Vaes B L, Ducy P, Sijbers A M, Hendriks J M, van Someren E P, de Jong N G, van den Heuvel E R, Olijve W, van Zoelen E J, Dechering K J 2006 Microarray analysis on Runx2-deficient mouse embryos reveals novel Runx2 functions and target genes during intramembraneous and endochondral bone formation. Bone 39:724-738
45. Kousteni S, Bellido T, Plotkin L I, O'Brien C A, Bodenner D L, Han L, Han K, DiGregonio G B, Katzenellenbogen J A, Katzenellenbogen B S, Roberson P K, Weinstein R S, Jilka R L, Manolagas S C 2001 Nongenotropic, sex-nonspecific signaling through the estrogen or androgen receptors: dissociation from transcriptional activity. Cell 104:719-730
46. Castro-Rivera E, Samudio I, Safe S 2001 Estrogen regulation of cyclin D1 gene expression in ZR-75 breast cancer cells involves multiple enhancer elements. J Biol Chem 276:30853-30861
47. Sims N A, Clement-Lacroix P, Minet D, Fraslon-Vanhulle C, Gaillard-Kelly M, Resche-Rigon M, Baron R 2003 A functional androgen receptor is not sufficient to allow estradiol to protect bone after gonadectomy in estradiol receptor-deficient mice. J Clin Invest 111:1319-1327
48. Sims N A, Dupont S, Krust A, Clement-Lacroix P, Minet D, Resche-Rigon M, Gaillard-Kelly M, Baron R 2002 Deletion of estrogen receptors reveals a regulatory role for estrogen receptors-beta in bone remodeling in females but not in males. Bone 30:18-25
49. Pearce S T, Jordan V C 2004 The biological role of estrogen receptors alpha and beta in cancer. Critical reviews in oncology/hematology 50:3-22
50. Duss S, Andre S, Nicoulaz A L, Fiche M, Bonnefoi H, Brisken C, Iggo R D 2007 An oestrogen-dependent model of breast cancer created by transformation of normal human mammary epithelial cells. Breast Cancer Res 9:R38
51. Kawate H, Wu Y, Ohnaka K, Takayanagi R 2007 Mutual transactivational repression of Runx2 and the androgen receptor by an impairment of their normal compartmentalization. The Journal of steroid biochemistry and molecular biology 105:46-56
52. Jilka R L, Takahashi K, Munshi M, Williams D C, Roberson P K, Manolagas S C 1998 Loss of estrogen upregulates osteoblastogenesis in the murine bone marrow. Evidence for autonomy from factors released during bone resorption. J Clin Invest 101:1942-1950
53. van de Vijver M J, He Y D, vanet Veer L J, Dai H, Hart A A, Voskuil D W, Schreiber G J, Peterse J L, Roberts C, Marton M J, Parrish M, Atsma D, Witteveen A, Glas A, Delahaye L, van der Velde T, Bartelink H, Rodenhuis S, Rutgers E T, Friend S H, Bernards R 2002 A gene-expression signature as a predictor of survival in breast cancer. N Engl J Med 347:1999-2009
54. Desmedt C, Sotiriou C 2006 Proliferation: the most prominent predictor of clinical outcome in breast cancer. Cell Cycle 5:2198-2202
55. Vaes B L, Ducy P, Sijbers A M, Hendriks J M, van Someren E P, de Jong N G, van den Heuvel E R, Olijve W, van Zoelen E J, Dechering K J 2006 Microarray analysis on Runx2-deficient mouse embryos reveals novel Runx2 functions and target genes during intramembraneous and endochondral bone formation. Bone 39:724-738
56. Ducy P, Karsenty G 1995 Two distinct osteoblast-specific cis-acting elements control expression of a mouse osteocalcin gene. Mol Cell Biol 15:1858-1869
57. Young D W, Hassan M Q, Yang X Q, Galindo M, Javed A, Zaidi S K, Furcinitti P, Lapointe D, Montecino M, Lian J B, Stein J L, van Wijnen A J, Stein G S 2007 Mitotic retention of gene expression patterns by the cell fate-determining transcription factor Runx2. Proceedings of the National Academy of Sciences of the United States of America 104:3189-3194
58. Hecht J, Seitz V, Urban M, Wagner F, Robinson P N, Stiege A, Dieterich C, Kornak U, Wilkening U, Brieske N, Zwingman C, Kidess A, Stricker S, Mundlos S 2007 Detection of novel skeletogenesis target genes by comprehensive analysis of a Runx2(−/−) mouse model. Gene Expr Patterns 7:102-112
59. Lin C Y, Strom A, Vega V B, Kong S L, Yeo A L, Thomsen J S, Chan W C, Doray B, Bangarusamy D R, Ramasamy A, Vergara L A, Tang S, Chong A, Bajic V B, Miller L D, Gustafsson J A, Liu E T 2004 Discovery of estrogen receptor alpha target genes and response elements in breast tumor cells. Genome Biol 5:R66

### Example II

### Repression of Runx2 by Androgen Receptor (AR) in Osteoblasts and Prostate Cancer Cells: AR Binds Runx2 and Abrogates its Recruitment to DNA

### Abstract

Runx2 and the androgen receptor (AR) are master transcription factors with pivotal roles in bone metabolism and cancer progression. We dissected AR-mediated repression of Runx2 in dihydrotestosterone (DHT)-treated osteoblastic and prostate cancer (PCa) cells using reporter assays and the endogenous Runx2 target gene, osteocalcin (OC). Repression required DHT, but not AR's transactivation function, and was associated with nuclear co-localization of the two proteins. Runx2 and AR co-immunoprecipitated and interacted directly in GST pull-down assays. Interaction was ionic in nature. Intact AR DNA-binding domain (DBD) was necessary and sufficient for both interaction with Runx2 and its repression. Runx2 sequences required for interaction were the C-terminal 132 amino acid residues together with the Runt DBD. Runx2 DNA-binding was abrogated by endogenous AR in chromatin immunoprecipitation assays and by recombinant AR-DBD in electromobility shift assays. Furthermore, AR caused increased nuclear mobility of Runx2-GFP as indicated by faster fluorescence recovery after photobleaching. Thus, AR binds Runx2 and abrogates its binding to DNA and possibly to other nuclear components. Clinical relevance of our results is suggested by an inverse correlation between expression of the AR responsive prostate-specific antigen (PSA) and OC genes in PCa biopsies. Given the tumor suppressor properties of Runx2, its repression by AR may constitute a mechanism of hormone carcinogenesis. In bone, attenuation of Runx2 by AR may restrain bone resorption and prevent high-turnover bone loss such as seen after orchidectomy and in transgenic mice whose osteoblasts over-express Runx2.

### Introduction

Runx2 is one of the three mammalian transcription factors sharing the highly conserved DNA-binding Runt domain. It is best known for its mandatory role in osteoblast and chondrocyte differentiation from mesenchymal precursors (1-4). In addition to promoting embryonic bone formation, Runx2 plays a role in postnatal bone metabolism and the control of bone mass (5-9). Runx2 is also expressed in breast and prostate cancer cells, possibly contributing to their tumorigenicity and metastatic potential, in part by stimulating the expression of matrix metalloproteases (10, 11). In other contexts, the Runx proteins, including Runx2, function as tumor suppressors (12, 13).

The most well established target gene for Runx2 is osteocalcin (OC). The Osteoblast Specific Element 2 (OSE2) at the OC promoter served as a molecular tool by which Runx2 was discovered as an osteoblast master transcription factor (5, 14). However, OC does not mediate the role of Runx2 in osteoblast differentiation and bone metabolism (15, 16). Many other Runx2 target genes in osteoblasts have also been identified (17-20), but most of them have not been tested yet for their roles in bone cell function. Even less is known about target genes that mediate the tumor suppressor function of Runx2. Notable candidates are the cyclin-dependent kinase inhibitor p21 (21) and the pro-apoptotic protein Bax (22).

In addition to the Runt domain, Runx2 has an N-terminal glutamine-alanine (Q/A) domain and a C-terminal proline-serine-threonine (PST) domain. Within the PST domain are Runx2's nuclear localization and the nuclear matrix targeting signals (NMTS) (23). A number of co-regulatory proteins interact with, and orchestrate Runx2 activities. Binding of Cbfβ to the Runt domain enhances the affinity of Runx2 to OSE2-like response elements and is required for proper control of skeletogenesis (24). Runx2, via its PST domain, recruits co-activators such as p300, MOZ and MORF (25, 26), as well as co-repressors such as TLE/Groucho, HDAC6 and YAP, to activate or repress target genes in a context-dependent manner (21, 27). Of particular interest, the positive role of osteogenic BMPs and the negative role of TGFβ in skeletal development are mediated in part by interaction of their signal transducers (SMAD 1, 5 and 8 and SMAD 2 and 3, respectively) with Runx2 (28, 29). Runx2 expression and activity are also controlled at the levels of transcription (30), translation (31), post-translational modifications (32), nuclear and sub-nuclear localization (33) and access to chromatin-embedded targets (34). Interestingly, Runx2's SMAD interaction domain (SMID) overlaps with its NMTS (28, 35). There is also evidence that growth factors influence the function of bone and other cell types in part by MAP kinase-mediated phosphorylation of residues in Runx2's PST domain (36-38).

Like Runx2, the androgen receptor (AR) is also a transcription factor that plays important roles in bone and cancer biology. It is crucial for the survival and growth of both androgen-dependent and ablation-resistant prostate cancer (PCa) cells (39-41), and its target gene prostate-specific antigen (PSA) is frequently used as a surrogate for AR activity in clinical settings and to study its mechanisms of action (42). Similar to other steroid hormone receptors, AR contains an N-terminal domain (NTD) that contains activation function-1 (AF1), a conserved zinc-finger DNA-binding domain (DBD), and a ligand-binding domain (LBD) that contains activation function-2 (AF2). Testosterone or its more potent derivative dihydrotestosterone (DHT) bind to the LBD to induce a conformational change that triggers AR nuclear translocation and transcription of target genes (43), which mediate the hormone's roles in diverse physiological and disease processes.

The proskeletal properties of androgens include promotion of bone formation and attenuation of bone resorption. This is achieved both via aromatization to estrogens, which then activate estrogen receptor-α (ERα), and by aromatization-independent mechanisms (44-47). Lack of androgens or AR results in progressive loss of bone mass and increased risk of fracture (46, 48-50). Although AR is clearly implicated in the regulation of bone cell function, the underlying molecular mechanisms remain unclear.

We recently demonstrated that ERα interacts with and modulates Runx2 activity in osteoblasts and breast cancer (BCa) epithelial cells (51). Attenuation of Runx2 by ERα likely contributes to the beneficial effect of estrogens in the adult skeleton because unrestrained Runx2 in osteoblasts leads to increased osteoclast activation and loss of bone mass (7, 52). In BCa cells, inhibition of Runx2 activity by estrogens may promote carcinogenesis given the tumor suppressor properties of Runx proteins (12, 13). In the present study, we dissected the regulation of Runx2 activity by AR in osteoblastic and prostate cancer cell lines. We report that, like ERα, AR interacts with Runx2 and inhibits its activity. However, the two receptors bind different regions of Runx2. Of particular importance, whereas both receptors require the PST domain of Runx2 for interaction, only the AR requires, in addition, the Runt domain, interaction with which diminishes the DNA-binding function of Runx2.

### Results

### AR Represses Runx2 Independent of its Own Transcriptional Transactivation Activity

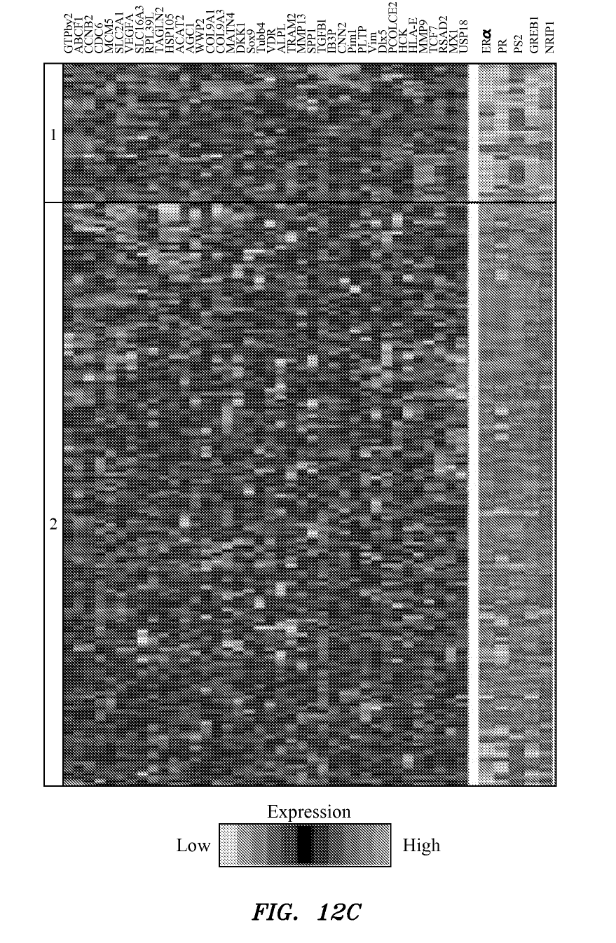

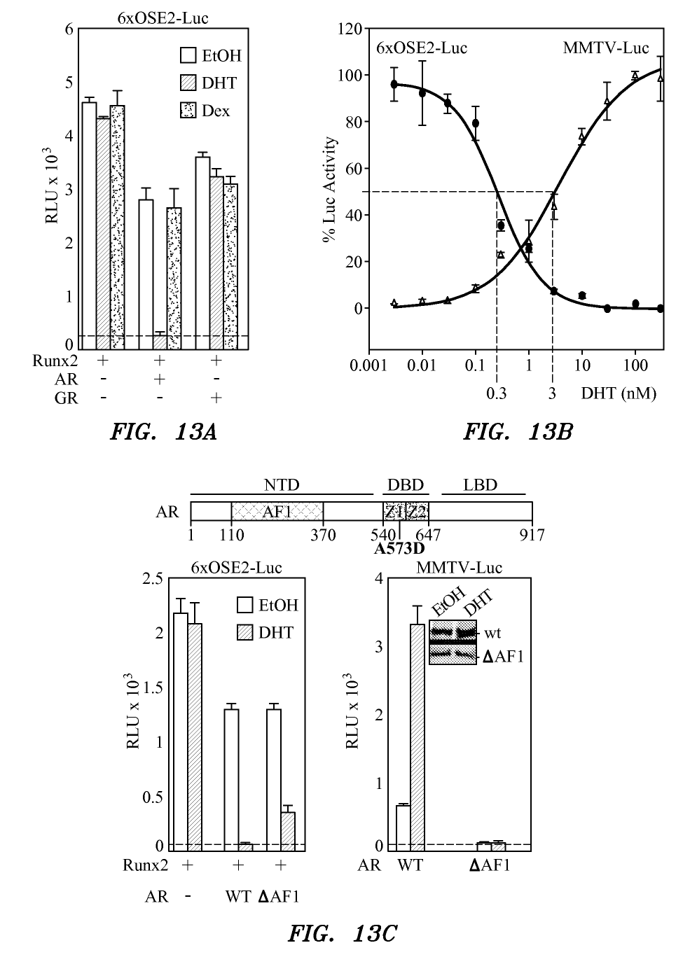

We examined the effect of AR on the transcriptional potential of Runx2 using transient transfection reporter assays COS7 cells, which express neither transcription factor, were transfected with plasmids encoding Runx2 and AR, along with the 6XOSE2-Luc reporter, in which luciferase is controlled by six copies of the OSE2 Runx2 binding site (53). The reporter had very low basal activity and was strongly activated upon Runx2 expression (FIG. 13A). In the absence of AR, its ligand dihydrotestosterone (DHT) did not affect Runx2 activity. However, DHT treatment in the presence of AR repressed Runx2 activity to basal levels (FIG. 13A). DHT did not alter Runx2 activity in the presence of the glucocorticoid receptor (GR), neither did dexamethasone (DEX) in the presence of GR or AR (FIG. 13A). Thus, repression of Runx2 by DHT is both receptor and ligand specific.

Since AR is a potent transcription factor, we investigated if its transactivation function was required for repressing Runx2. First, we took advantage of the fact that ligand was required for both the specific AR-mediated transactivation function and its Runx2 repression activity, and derived DHT dose-response curves for each function (FIG. 13B). MMTV-Luc and 6XOSE2-Luc were used as reporters for monitoring the transactivation and Runx2 repression functions of AR, respectively, with equal amounts of each of AR and Runx2 expression plasmids co-transfected in both assays. As shown in FIG. 13B, the DHT concentration required to achieve 50% Runx2 repression was ten fold lower than that required for 50% AR transactivation. Thus, AR-mediated Runx2 repression occurs more readily than AR-mediated transactivation, suggesting that Runx2 repression does not require stimulation of direct AR targets gene(s).

To further clarify the dissociation between AR's transactivation and Runx2 repression functions, we tested an AR mutant that lacks the AF1 domain (AR^{Δ}^{_{AF1}}) (FIG. 13C, block diagram). DHT-bound AR^{Δ}^{_{AF1}} repressed Runx2 similar to the wild type (WT) AR (FIG. 13C, left panel) even though it was transcriptionally inactive (FIG. 13C, right panel). These results strongly suggest that the transactivation function of AR is not required for Runx2 repression.

### Physical Interaction Between AR and Runx2: AR DNA-Binding Domain (DBD) is Necessary and Sufficient for Runx2 Repression

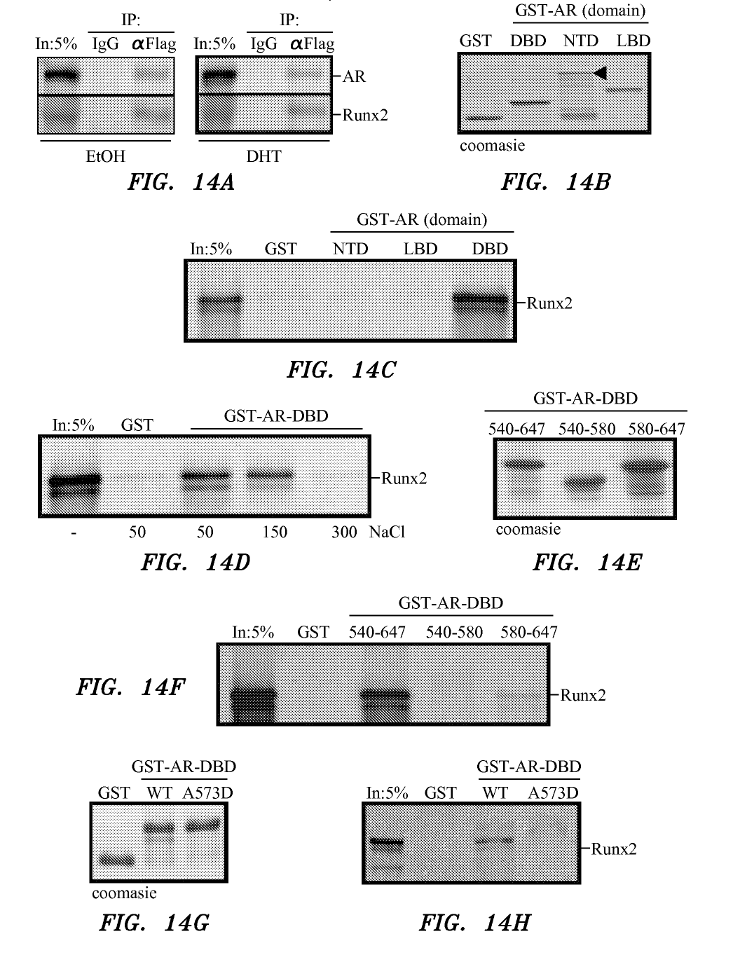

Repression of Runx2 by AR could be mediated by direct interaction between the two proteins as previously observed in GST pull-down assays (54). We first confirmed the interaction between AR and Runx2 transiently expressed in COS7 cells. The cells were treated with DHT or vehicle and subjected to co-immunoprecipitation (co-IP) assays. As shown in FIG. 14A, AR was present in Runx2 immunoprecipitates, suggesting that the strong inhibition of Runx2 in COS7 cells could indeed involve protein-protein interaction. However, given that the inhibition was largely ligand-dependent (FIG. 13A), we were initially surprised that the interaction in the co-IP assay was ligand-independent (FIG. 14A). This was later clarified in our intracellular localization studies (see below).

To determine which part of AR participates in the interaction with Runx2, we performed GST pull-down assays with the three major domains of AR (FIG. 13C, block diagram) as baits. The prey was in vitro transcribed and translated full-length Runx2. Of the three baits (FIG. 14B), only the AR-DBD bound Runx2 (FIG. 14C). Increasing concentrations of NaCl inhibited the AR-DBD/Runx2 binding (FIG. 14D), suggesting that the interaction was of ionic nature. Next we examined the contribution of the two zinc fingers constituting the AR-DBD to the interaction. GST-tagged fragments containing the individual zinc fingers, amino acid (aa) residues 540-580 and 580-647 (FIG. 14E), were tested for binding to Runx2. Unlike the intact AR-DBD (aa 540-647), none of these fragments bound Runx2 (FIG. 14F), indicating that structural integrity of the whole AR-DBD is crucial for the interaction. We further substantiated specificity of our results by using an AR-DBD mutant, in which a highly conserved alanine residue within the first zinc finger (position 573) is replaced by aspartic acid (AR-DBD^{A573D}). This mutation disrupts the AR DNA-binding activity and causes Reifenstein Syndrome. In contrast to its WT counterpart, GST-AR-DBD^{A573D} (FIG. 14G) failed to pull-down Runx2 (FIG. 14H), corroborating our conclusion that an intact AR-DBD is mandatory for binding Runx2. Finally, given the extreme specificity of the AR-DBD/Runx2 interaction, we investigated whether the A573D mutation abrogated Runx2 repression in transient transfection assays. As shown in FIG. 14I, AR^{A573D} did not mediate the repression of Runx2 as observed with the WT receptor. These results implicate the physical interaction between AR-DBD and Runx2 (FIG. 14C) in AR-mediated Runx2 repression (FIG. 13A).

Although AR-DBD is sufficient for binding Runx2, it may not be sufficient for executing the repression function. We therefore tested the ability of the AR-DBD to repress Runx2. Remarkably, transiently expressed AR-DBD repressed Runx2 as effectively as the full-length receptor (FIG. 14I). As expected, the repression was DHT-independent. As control, AR-NTD, which did not bind Runx2 (FIG. 14C), did not influence Runx2 activity (FIG. 14I). Thus, the structurally intact AR-DBD is necessary and sufficient for both binding to and repressing Runx2.

AR Colocalizes with and Alters the Mobility of Runx2 in Living Cells

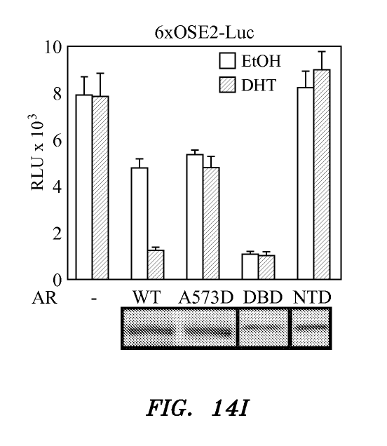

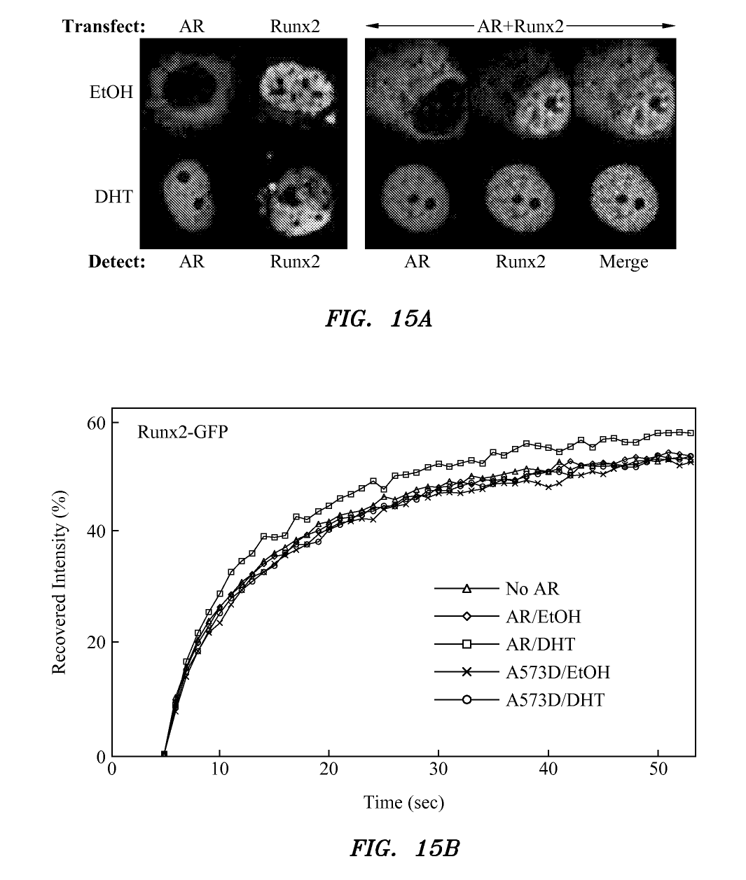

The interaction and behavior of AR and Runx2 in living cells were next investigated by confocal microscopy. First, using indirect immunofluorescence, we visualized the intracellular localization of AR and Runx2 that were transiently expressed in COS7 cells in the presence of DHT or vehicle. Whether expressed alone or together, AR was primarily cytoplasmic and became nuclear upon DHT treatment, whereas Runx2 was nuclear irrespective of treatment (FIG. 15A) Merged confocal images showed that the two transcription factors colocalize in the COS7 cell nuclei only after DHT treatment (FIG. 15A), offering a potential explanation for hormone requirement in the functional inhibition assays (FIG. 13A) despite the hormone-independent interaction in cell homogenates (FIG. 14A). The DHT-mediated colocalization was associated with alterations in the patterns of AR and Runx2 sub-nuclear distribution. Similar to the recent observations of Kawate et al. (55), the AR and Runx2 colocalized in subnuclear structures, which were only observed when the two transcription factors were co-expressed and the cells treated with DHT (FIG. 15A).

Interaction of transcription factors with other nuclear components is often linked to their intra-nuclear mobility (56, 57). We employed fluorescence recovery after photobleaching (FRAP) to examine the mobility of Runx2 in response to AR activation. COS7 cells expressing Runx2-GFP, alone or together with AR, were treated with either DHT or vehicle, and a small nuclear section was briefly photobleached, followed by measurements of the fluorescence recovery over time. As shown in FIG. 15B, the Runx2-GFP repopulated the photobleached section faster in the presence of DHT-bound AR as compared to Runx2-GFP alone or in the presence of unliganded AR. Furthermore, the AR^{A573D} mutant, which does not interact with Runx2 (FIG. 14H), did not significantly influence the mobility of Runx2-GFP (FIG. 15B). These results provide further evidence for in vivo interaction between AR and Runx2, and suggest that the AR competes out another, relatively immobile nuclear component that otherwise engages Runx2 (see discussion).

AR Interacts with and Inhibits Runx2 in Prostate Cancer Cells

Because Runx proteins, including Runx2, possess tumor suppressor properties (12, 13), inhibition of Runx2 by AR may play a role in prostate carcinogenesis. We chose to study the interaction between these two transcription factors in the PC3 PCa cell line, which exhibited the highest Runx2 activity among several PCa cell lines tested. The AR, which is not expressed in these cells (58), was initially introduced by transient transfection, and was visualized along with the endogenous Runx2 using immunofluorescence confocal microscopy. In contrast to COS7 cells, where transiently expressed Runx2 was localized in the nucleus (FIG. 15A), the endogenous Runx2 in PC3 cells was nucleo-cytoplasmic (FIG. 16A). Without DHT, transiently expressed AR displayed a similar nucleo-cytoplasmic localization. Remarkably, treatment with DHT led to nuclear localization of not only AR, but also Runx2 (FIG. 16A). Because DHT did not influence Runx2 localization in the absence of AR (FIG. 16A), it is most likely the physical interaction between the two proteins that led to the DHT-induced nuclear localization of Runx2. Furthermore, merged images revealed a strong co-localization of AR and Runx2 in PC3 cells (FIG. 16A) and, similar to the co-IP results from COS7 cells, the two proteins were co-immunoprecipitated from PC3 cell homogenates. These results suggest that AR physically interacts with and inhibits Runx2 activity in PCa cells.

To study the influence of AR on Runx2 activity and on the Runx2 target gene OC in PCa cells, AR was first stably introduced into PC3 cells by retroviral infection. The transduced cells, designated PC3-AR, presumably resemble PCa cells that endogenously express both Runx2 and wild type AR. These cells were transiently transfected with the MMTV-Luc or the 6XOSE2-Luc reporter plasmid. PC3-GFP cells, transduced with a GFP-encoding retrovirus, were used as control. PC3-AR, but not PC3-GFP cells showed robust MMTV-Luc activity in response to DHT, indicating expression of functional AR (FIG. 16B, left panel). The 6XOSE2-Luc reporter was strongly expressed in both PC3-GFP and PC3-AR cells indicating the presence of endogenous functional Runx2. As expected from our studies in COS7 cells, DHT treatment led to a strong repression of the 6XOSE2-Luc reporter activity in PC3-AR but not in PC3-GFP cells (FIG. 16B, middle panel). To test whether our plasmid-based reporter assays faithfully represented the activity of Runx2 in the context of native chromatin, we analyzed the effect of DHT on the expression of OC in both PC3-AR and PC3-GFP cells. RT-qPCR analyses showed that DHT treatment strongly inhibited OC expression in PC3-AR but not in PC3-GFP cells (FIG. 16B, right panel). Thus, AR inhibits Runx2 activity and expression of the Runx2 target gene OC in PCa cells.

We further investigated the relationship between AR activation and OC expression in a series of 40 PCa primary tumors, 23 from patients undergoing resection of the primary tumor without androgen ablation therapy (AAT), and 17 from patients who had initiated AAT three months prior to tumor resection. These tumors, which provide a wide range of AR activation levels, were previously subjected to comprehensive microarray-based analysis of gene expression (59). From the microartay data, we extracted the values of PSA and OC mRNAs as measures of AR and Runx2 activity, respectively. As shown in FIG. 16C, expression of OC negatively correlated with that of PSA whether or not the patients underwent AAT. A statistically significant negative correlation between PSA and OC expression (R=−0.53, p=0.002) was also observed when the two groups were analyzed together. These results may be driven at least in part by the coordinated AR-mediated transcriptional stimulation and Runx2 repression functions, as demonstrated in the PC3 culture model (FIG. 10B). Given the tumor suppressor property of Runx2 (12, 13), our results are consistent with the hypothesis that AR-mediated inhibition of Runx2 in prostate epithelial cells contributes to the well-established role of AR in PCa initiation and progression.

### Androgens Inhibit Runx2 During Late Osteoblast Differentiation

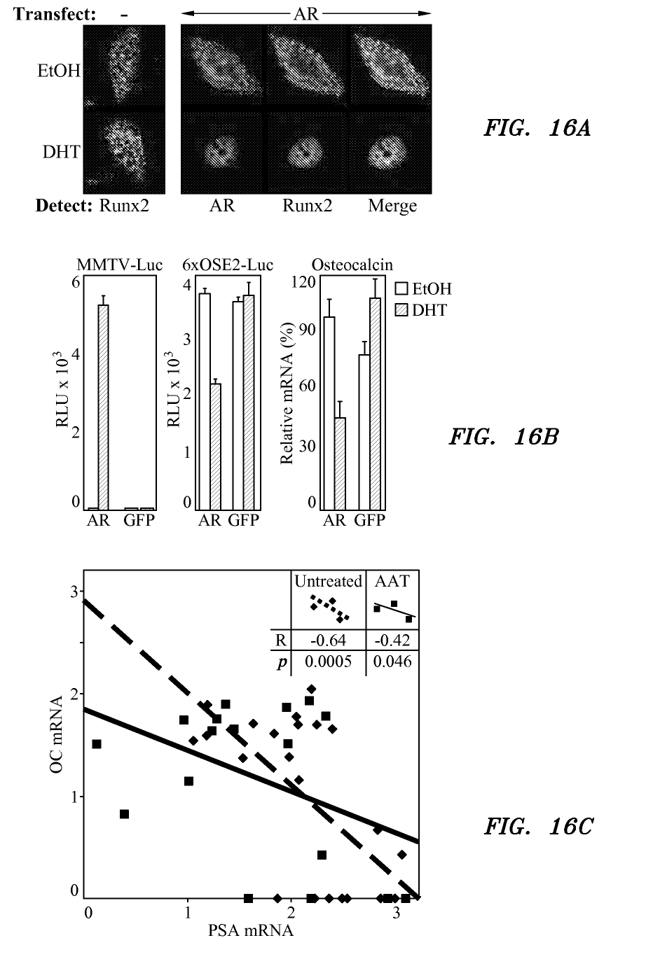

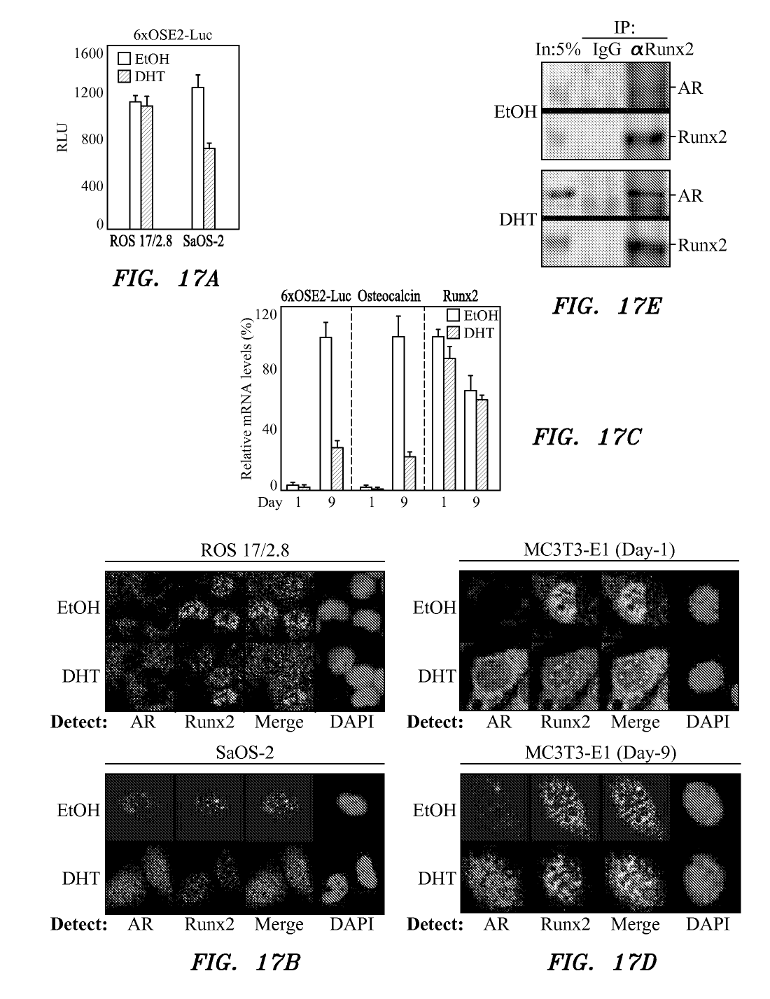

Manipulation of Runx2 in osteoblasts strongly influences bone turnover and bone mass, so modulation of Runx2 activity by AR in osteoblasts may explain some of the effects of androgens in bone. To address this, we initially transiently transfected SaOS-2 and ROS 17/2.8 osteosarcoma cells, which express endogenous Runx2 and AR, with the 6XOSE2-Luc reporter, followed by DHT treatment and luciferase assays. As shown in FIG. 17A, DHT inhibited luciferase activity in SaOS-2 but not in ROS 17/2.8 cells. This difference could be explained by the results of our confocal immunofluorescence microscopy studies (FIG. 17B): treatment of SaOS-2 cells with DHT resulted in nuclear translocation of AR, and its colocalization with Runx2. In contrast, DHT treatment did not drive the AR to the nucleus of ROS 17/2.8 cells, avoiding colocalization with Runx2 (FIG. 17B). Conceivably, cytoplasmic retention of the AR preserved Runx2 activity in DHT-treated ROS 17/2.8 cells.

To test whether AR inhibits Runx2 in non-tumorigenic osteoblasts, we turned to the MC3T3-E1 pre-osteoblast-like cell line, which also expresses both Runx2 and AR. A sub-line that had been stably transfected with the 6XOSE2-Luc reporter (51) was employed to simultaneously test the influence of AR on pure Runx2 activity (luciferase) and on OC gene expression. The cells were treated with DHT for 24 hrs commencing at confluence (day 0) and subjected on days 1 and 9 to RT-qPCR analysis of the mRNAs encoding luciferase and OC. As shown in FIG. 17C, expression of both 6XOSE2-Luc and OC was very low in the early cultures (day 1) and dramatically increased in late cultures (day 9). On day 9, expression of both 6XOSE2-Luc and OC was strongly inhibited by DHT, whether introduced chronically since confluence (FIG. 17C) or for only 24 hrs prior to harvest. Thus, AR inhibits Runx2 in osteoblasts during late stages of their developmental program.

Immunofluorescence analysis of day-1 and day-9 MC3T3-E1 cultures provided interesting insights into the expression and localization of AR and Runx2 in response to DHT. First, similar to previous observations in osteoblasts (60, 61), DHT strongly induced AR protein levels in both the early and the late cultures (FIG. 17D). Second, unlike the classical paradigm, AR remained primarily cytoplasmic in early cultures even after the DHT treatment. The more conventional AR behavior—nuclear localization in the presence of DHT—was observed only in the late cultures (FIG. 17D). The intracellular distribution of Runx2 also strongly depended on culture maturity. Without DHT treatment, most of the Runx2 on day 1 was nuclear and was found in numerous sub-nuclear foci. On day 9, Runx2 was more evenly distributed and occupied lesser sub-nuclear foci (FIG. 17D). Treatment of the early cultures with DHT resulted in redistribution of Runx2, a larger fraction of which became cytoplasmic and strongly colocalized with the AR (FIG. 17D). In the late cultures, Runx2 became nuclear after DHT treatment and the hormone directed the distribution of Runx2 into sub-nuclear domains that resembled those occupied by the AR (FIG. 17D).

Finally, we examined the interaction between AR and Runx2 in co-IP assays of Day-9 MC3T3-E1 cultures. Runx2 was immunoprecipitated and the complexes were subjected to Western blot analysis of AR. As shown in FIG. 17E, AR was present in immunocomplexes obtained with anti-Runx2 antibodies, but not with non-specific IgG. Similar to the results with COS7 (FIG. 17A) and PC3 cells, the interaction did not require hormone treatment in the co-IP assay. Thus, DHT inhibits Runx2 during late stages of osteoblast differentiation most likely via nuclear translocation of the AR that leads to direct interaction with Runx2 (also see FIG. 17).

Runx2's PST and Runt Domains are Necessary for Interaction with AR

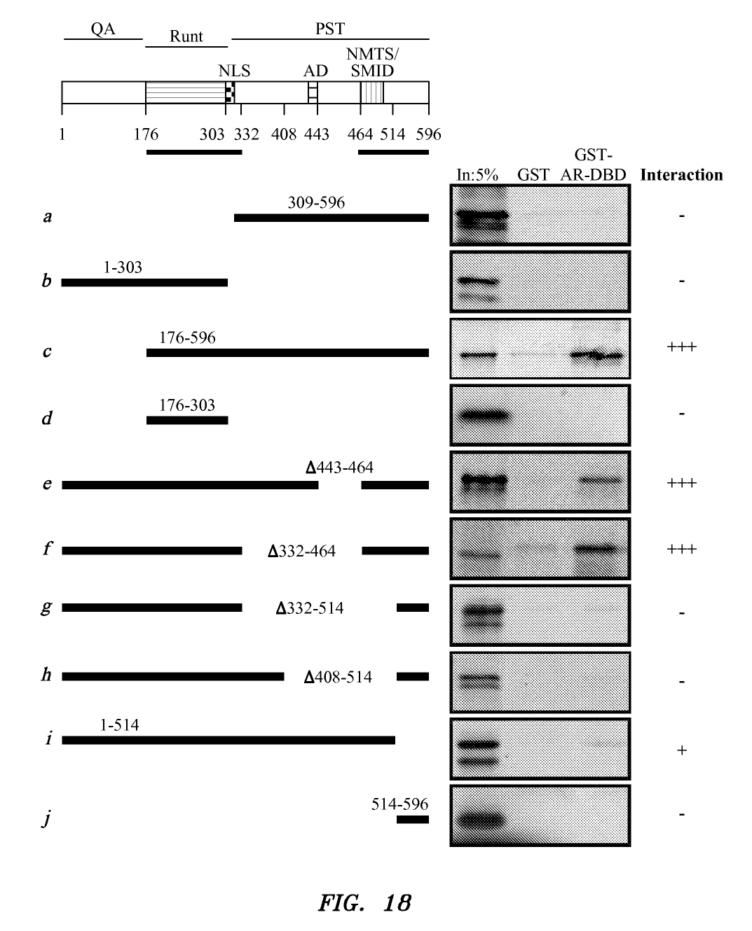

To further dissect the mechanism by which AR inhibits Runx2's transcriptional activity, we first mapped, using GST pull-down assays, the Runx2 sequences that mediate the interaction with the AR. AR-DBD, which is this receptor's domain that contacts Runx2 (FIG. 17C), was used as bait, and the Runx2 fragments illustrated in FIG. 18 were synthesized in vitro as ³⁵S-labeled preys. Because the interaction between Runx2 and ERα was recently mapped to the ERα-DBD and Runx2's PST domain (51), and given the high conservation between AR-DBD and ERα-DBD, we first tested whether Runx2's PST domain interacted with AR-DBD. Surprisingly, GST-AR-DBD failed to pull-down Runx2's PST domain (FIG. 18a). Similarly, no binding was observed with a fragment representing the QA and Runt domains of Runx2 (FIG. 18b). However, a Runx2 fragment containing both the PST and the Runt domains effectively recapitulated the interaction observed with full-length Runx2 (FIG. 18c). Like the PST domain, the Runt domain alone also did not bind AR-DBD (FIG. 18d). Thus, both the Runt and the PST domains were necessary but neither one alone was sufficient for the interaction with AR-DBD.

To delineate the minimal PST sequence necessary for the interaction with AR-DBD, we tested a series of internal deletions in the PST domain (FIGS. 18e-h). Binding was detectable with mutant Δ443-464 (FIG. 18e), which retained the activation domain (AD) and the NMTS/SMID domain. Strong interaction was also observed with mutant A332-464 despite absence of the AD (FIG. 6f). However, the mutants Δ332-514 and Δ408-514 (FIGS. 18g and h), which lacked the NMTS/SMID domain did not bind the AR-DBD. Finally, the deletion mutant 1-514 (FIG. 18i), which retained both the Runt and the NMTS/SMID domains, but lacked the very C-terminal 82 aa residues of Runx2, bound AR-DBD only weakly, suggesting that the 82 C-terminal aa residues are critical for interaction with the AR-DBD. However, this 82 aa fragment did not bind to AR on its own (FIG. 18j). Thus, the Runt domain and the C-terminal part of the PST domain (aa 464-596, from the NMTS/SMID to the C-terminus) together represent the minimal Runx2 sequence necessary for binding the AR-DBD (FIG. 18, black lines under the block diagram).

AR Displaces Runx2 from its OSE2 Target Site at the OC Promoter

Because Runx2's Runt domain is necessary for both DNA binding (62) and interaction with the AR-DBD (FIG. 18), we hypothesized that the AR-DBD prevents Runx2 from binding DNA, accounting for its repression activity. This was initially tested by examining the effect of AR-DBD on the binding of Runx2 to DNA in EMSA with a ³²P-labeled OSE2 probe. The source for Runx2 was MC3T3-E1 cell extract and recombinant GST-AR-DBD was purified from E. coli. Of the two complexes formed, the slow-migrating complex contained Runx2, as it was sensitive to the presence of Runx2 antibodies (FIG. 19A, lane 2 Vs 8), but not to the same antibodies that were denaturated by boiling (FIG. 19A, lane 9). Formation of this Runx2/OSE2 complex was progressively inhibited by increasing amounts of GST-AR-DBD (FIG. 19A, lanes 2-4), but not by GST alone (FIG. 19A, lane 6). GST-AR-DBD itself did not form any complex with the OSE2 probe (FIG. 19A, lane 7), suggesting that it did not inhibit the Runx2/DNA interaction by competing for a common binding site. Unlike GST-AR-DBD, GST-AR-DBD^{A573D}, which does not interact with Runx2 (FIG. 14F), failed to inhibit the Runx2/DNA complex formation (FIG. 19A, lane 5). These results strongly suggest competition for the Runt domain as the mechanism underlying AR-mediated repression of Runx2.

To test whether the DNA-binding exclusion model operates in vivo, we investigated whether AR interferes with binding of Runx2 to its chromatin-embedded OSE2 element at the OC promoter in MC3T3-E1 cells. Day-9 cultures, in which Runx2 occupancy at this site is maximal (34), were treated for four hours with DHT and subjected to Runx2 ChIP assay. As shown in FIG. 19B, Runx2 occupancy of the endogenous OSE2 site decreased by 2.4-fold in response to DHT treatment. The inhibition of Runx2 occupancy occurred without any significant change in Runx2 protein levels (FIG. 19B, inset). Altogether, the GST pull-down, the EMSA, the immunofluorescence analyses, and the ChIP data suggest that AR binding to Runx2 abrogates its recruitment to target genes promoters. The mutually exclusive interaction of Runx2 with either DNA or AR could account for its repression by androgens.

### Discussion

Besides its physiological functions in the male reproductive system, the AR has important regulatory roles in prostate cancer and bone metabolism. AR's function has been primarily attributed to transcriptional stimulation of target genes via AREs located in their promoters and enhancers. The strong inhibition of Runx2 by AR, dissected in the present study, may constitute an additional significant mechanism through which androgens execute their functions. Consistent with our in vitro observations, in vivo expression of OC, a classical Runx2 target gene, was inhibited in mice whose osteoblasts over-express AR (63) and in testosterone-repleted versus experimentally-hypogonadal men (44), and, OC expression was stimulated in AR knock-out mice (46). In our studies, the inhibition of Runx2 activity was observed in a variety of cell lines, irrespective of whether the target was a transiently transfected reporter plasmid, a stably-integrated reporter, or the endogenous OC target gene. The inhibition of Runx2 did not require AR-induced transcription. Three lines of evidence support this conclusion: (i) the inhibition occurred at concentrations an order of magnitude lower than those inducing transcription; (ii) the inhibition was observed with the transcriptionally-inactive AR^{ΔAF1} mutant; and (iii) even the AR-DBD alone, which is the receptor's domain that interacts with Runx2, was sufficient for inhibition.

None of Runx2's three major domains is sufficient for interaction with the AR. Instead, both the Runt domain and aa residues 464-596 within the PST domain were necessary for binding AR. The involvement of the Runt domain raised a plausible mechanism, whereby AR prohibits Runx2 from binding DNA. Evidence in support of such a simple mechanism was provided by EMSA and ChIP assays, where the presence of recombinant AR-DBD or treatment with DHT disrupted Runx2 DNA-binding activity and its genomic recruitment, respectively. Interestingly, the Runx2 domains required for interaction with AR also serve as docking sites for other nuclear components. For example, the Runt domain interacts with CbMβ (24), and aa residues 464-596 encompass binding sites for the nuclear matrix and numerous coregulatory proteins including SMADs (28) and TLE/Groucho (64). We speculate that these nuclear components may influence the ability of AR to associate with Runx2 and may coordinate between androgen signaling and other pathways that impinge on Runx2. Additionally, post-translational modifications of the Runt domain or the 464-596 region, e.g., by MAP-kinase (37) may disrupt Runx2's interaction with and repression by the AR.

Similar to the AR, ERα as well as GR and the Vitamin D receptor (VDR) also interact with, and modulate Runx2 activity (51, 54, 65). The receptor's interaction domain was pursued for AR, ERα and VDR, and was mapped to the zinc finger-containing DBD in each of these cases. The A573D point mutation in AR-DBD and the S51G mutation in VDR-DBD (65), which compromise their respective structural integrity, also prevent their binding to Runx2. Interestingly, despite being highly homologous, each receptor's DBD recognized a distinct part of Runx2. This is possibly related to the different biological roles assigned for each of the interactions. For example, ERα and AR repress, whereas VDR activates Runx2 activity (51, 65).

The inhibition of Runx2 by AR is potentially important in many aspects of human health and disease, including PCa and bone biology. Like the other two members of the Runt family, Runx2 is implicated in the control of cellular carcinogenesis and plays a role as either tumor suppressor or enhancer in a context-dependent manner (13, 66). Conceivably, the pivotal role of AR in prostate cancer progression is mediated in part by counteracting the tumor suppressor activity of Runx2 in prostate epithelial cells. The interpretation of AR-mediated Runx2 repression is more difficult in the context of advanced disease, where Runx2 could promote the metastatic phenotype (67). The fact that virtually all PCa tumors retain AR expression suggest that metastatic PCa is less dependent on Runx2 than ER-negative breast cancer metastases, whose unopposed Runx2 likely promotes the aggressive bone destructive behavior of these tumors. Alternatively, anti-AR Runx2-protective mechanisms may evolve in advanced PCa, possibly by sub-cellular or sub-nuclear compartmentalization of the two transcription factors. Finally, it will be interesting to test whether blockade of AR-mediated transcription with selective androgen receptor modulators (SARMs) is associated with increased Runx2 activity. Given the pro-metastatic activity of Runx2 (67), such SARMs may actually worsen the prognosis of patients with advanced disease. SARMs that block AR's transcriptional activity while maintaining its Runx2 repression function presents an attractive therapeutic approach for deadly ablation-resistant metastatic PCa.

Because Runx2 knockout experiments established this protein as a master transcription factor required for osteoblast differentiation (1, 2), the inhibition of Runx2 by estrogens (51) and androgens [(55) and the present work] may come as a surprise considering the strong protective effect of gonadal steroids on the skeleton (68). A solution for this dilemma is offered by work from two groups, which manipulated Runx2 using other approaches (3, 6-8). In each of three transgenic lines over-expressing Runx2 in osteoblasts, Matthias and colleagues (7) observed severe bone loss and spontaneous fractures associated with up to 10-fold increased endosteal osteoclast surface. Furthermore, using co-culture assays, this group demonstrated that Runx2-over-expressing osteoblasts isolated from the transgenic mice promoted supraphysiological osteoclastogenesis. In line with these results, Komori and colleagues showed that calvarial cells from Runx2 deficient mice fail to support osteoclast differentiation, unless Runx2 is virally restored (69). More recently, this group transduced normal calvarial osteoblasts with adenoviruses encoding wild type or dominant-negative Runx2, which resulted, respectively, in stimulated or repressed differentiation of co-cultured osteoclasts (8). These in vitro results lead to the same conclusion as that of Matthias and colleagues (7): over-expression of Runx2 in osteoblasts leads to exaggerated osteoclastogenic signal. In fact, Komory and colleagues (6) were the first to report on increased osteoclast activity at the endosteal surface of transgenic mice whose osteoblasts over-express Runx2. Although this early study focused on the defective bone formation, the studies of Runx2 manipulation in vitro and in vivo altogether assign a clear role for Runx2 in regulating osteoblast-derived signals to osteoclasts. Based on these studies, and considering the inhibition of Runx2 by estrogens and androgens, it is believed that the pro-skeletal function of gonadal steroids is executed in part by keeping Runx2 in check, thereby restraining osteoblast-mediated osteoclastogenesis. Indeed, increased bone resorption is a hallmark of bone loss that follows sex steroid withdrawal (68).

Osteoporosis observed in men with mutations in the ERα or aromatase genes demonstrate that much of the pro-skeletal effects of androgens in males are mediated by aromatization to estrogens, which then activate ERα (70-72). However, the obvious sexual dimorphism of the skeleton suggests an aromatization-independent but AR-dependent role for androgens. The extent of AR versus ER-dependent contribution of androgens to men's bone health is a matter of debate (44, 73). Studies with AR knockout mice, however, suggest that the two are of roughly equal importance, and that the role of AR is primarily executed by attenuating bone turnover (46). The mechanism mediating this effect of AR in bone is unknown, but may very well involve the inhibition of Runx2 as described in the present paper. That androgens protect the skeleton beyond the effects attributable to aromatization may be related to Runx2-independent mechanisms or to different mechanisms employed by AR versus ERα for interaction with and inhibition of Runx2. In this regard, we note that the Runt domain is absolutely necessary for interaction with the AR (this paper), but not with ERα. There is also a clear difference between the sequences within the PST domain that are necessary for Runx2's interaction with each receptor. For example, the 82 C-terminal aa residues of Runx2 are necessary for interaction with AR but not with ERα (51). Additional observations suggesting a mechanistic difference between AR- and ERα-mediated inhibition of Runx2 include: (i) AR binds to Runx2 stronger than ERα in GST pull-down assays; and (ii) ERα, but not AR, requires ligand for interaction with Runx2 in co-IP assays.

In summary, we documented inhibition of Runx2 by AR in a variety of cell lines derived from PCa and bone. The inhibition does not require AR transactivation activity, but rather requires direct contact between the two transcription factors, which disrupts Runx2's DNA binding activity. Inhibition of Runx2 by androgens may constitute a mechanism of hormone carcinogenesis in the initiation of PCa. In bone, it may be critical for limiting turnover thereby preventing osteoporosis. However, manipulation of Runx2 as a potential therapeutic strategy for either PCa or osteoporosis will be challenging. In the skeleton, the beneficial outcome of inhibiting Runx2—restrained bone turnover—might be associated with compromised bone formation. In PCa, AR-blockade may be beneficial in inhibiting the expression of AR target oncogenes and in stimulating the expression of Runx2 target genes that mediate its tumor suppressor activity; however the same treatment may cause harm by promoting Runx2 target genes that facilitate the metastatic phenotype. Thus, cell type-dependent and developmentally-regulated effects of androgens and estrogens on Runx2 activity must be considered when devising selective receptor modulators for the prevention and treatment of osteoporosis, breast and prostate cancer.

### Materials and Methods

Reagents—DHT and DEX were purchased form Sigma-Aldrich (St. Louis, Mo.), dissolved in ethanol at a 100 μM stock concentration. Unless otherwise stated, these hormones were applied at a final concentration of 10 nM in 0.001% ethanol as the vehicle. Primary antibodies for Runx2 (sc-10758) and for AR (sc-816 for immunofluorescence, otherwise sc-7305/N-20), as well as horseradish peroxidase-conjugated secondary antibodies were from Santa Cruz Biotechnology (Santa Cruz, Calif.). Flag antibodies were from Sigma-Aldrich. Fluorochrome-conjugated secondary antibodies for confocal microscopy were obtained from Pierce Biotechnology (Rockford, Il). Expression vectors for human AR (pcDNA3.1-AR), human GR (pSG5-GR) and the MMTV-Luc AR reporter were previously described (74), as were the 6XOSE2 Runx2 reporter plasmid and the mouse Runx2 expression vector (51). All other expression plasmids were constructed using standard cloning procedures and confirmed by sequencing. Lentiviral plasmid pHRCMV-Hygro-GFP was kindly provided by Dr. M. Stallcup's lab at USC, CA and pHRCMV-Hygro-Flag-AR was constructed by replacing the GFP cassette in pHRCMV-Hygro-GFP with a cDNA encoding human Flag-tagged AR (75).

Cell culture—The mouse osteoblast cell line MC3T3-E1, its derivative with a stably transfected 6xOSE2-Luc reporter (51), and ROS 17/2.8 cells were maintained in minimal essential medium (α-MEM) supplemented with 10% fetal bovine serum (FBS). COS7 and SaOS-2 cells were maintained in Dulbeco Modified Eagle Medium (DMEM) with 5% FBS. PC3 cells, were originally obtained from ATCC and a subline described earlier (58) was employed in the present study and maintained in RPMI-1640 and 5% FBS. All cells were incubated at 37° C. in a humidified 5% CO₂ incubator and the media was changed every 48 hrs.

Lentiviral particle production and transduction—For packaging, the lentiviral expression plasmids were co-transfected into HEK293T cells along with helper plasmids pMD.G1 and pCMVΔR8.91 (76). Culture media containing viral particles were harvested after 48-72 hrs and used for transduction of PC3 cells in the presence of 8 μg/ml Polybrene (Millipore). After infection with GFP or Flag-AR expressing lentiviruses the transduced cells were selected with 50 μg/ml Hygromycin (Invitrogen, Carlsbad, Calif.).

Transient transfection, luciferase, co-IP and ChIP—These assays were performed essentially as previously described (34, 51). A slight modification was introduced for immunoprecipitation using MC3T3-E1 and PC3-AR cells. These cells were plated in a 150 mm dish in phenol-red free media supplemented with 5% CSS. The MC3T3-E1 cell culture medium was further supplemented, commencing at confluence, with 50 μg/ml ascorbic acid and 150 mM β-glycerophosphate.

Quantitative real time RT-PCR—Total RNA was isolated using Aurum Total RNA® kit (Bio-Rad, Hercules, Calif.) following the manufacturer's recommendations. One microgram of total RNA was reverse-transcribed using the Superscript III kit (Invitrogen) and the cDNA was subjected to real-time PCR amplification (RTqPCR) using iQ™ SYBR Green Supermix (Bio-Rad). Primers for RTqPCR of mouse transcripts were previously described (51) and primers for RTqPCR of human transcripts were as follows: OC forward, 5′-GGCAGCGAGGTAGTGAAGAG-3′; OC reverse, 5β-CTGGAGAGGAGCAGAACTGG-3′; GAPDH forward, 5′-GTCATGGGTGTGAACCATGAGA-3′; and GAPDH reverse, 5′-GGTCATGAGTCCTTCCACGATAC-3′.

GST pull-down assay—Bacterial expression constructs for all GST pull-down bait proteins were derived from pGEX-4T-1 (Amersham Biosciences, Freiburg, Germany) by in-frame fusion of cDNA fragments encoding the indicated protein domains. GST-fusion proteins were over-expressed in Escherichia coli BL21(DE3) cells (Stratagene, Amsterdam, The Netherlands) and purified with the GST purification module (Amersham Biosciences) according to the manufacturer's protocol. ³⁵S-Runx2 and fragments thereof was produced using TNT® T7 Quick Kit from Promega according to the manufacturer's protocol. The pull-down assay was conducted in interaction buffer containing 20 mM Tris (pH 7.5), 100 mM NaCl (unless otherwise stated), 5 mM MgCl₂, 0.01% Nonidet P-40, and Complete™ protease inhibitor Mix® Roche Diagnostics (77). Each bait (10 μg) was immobilized on glutathione-S-sepharose beads and then incubated with the indicated ³⁵S-Runx2 or its fragments. The beads were washed five times with the interaction buffer, and the bound Runx2 fragments were eluted with 1× sample buffer and analyzed by SDS-PAGE and autoradiography along with 5% input.

Confocal immunofluorescence microscopy—Cells were grown on 18 mm² cover-slips in 6-well plates for 24 hrs in growth medium with CSS. Following transfection and hormone treatment, the cells were fixed with 95% methanol for 15 min and incubated with AR or Runx2 antibodies (1:500) followed by a rhodamine- or fluorescein-conjugated secondary antibody, respectively. Cells were mounted using Vectashield Hard Set mounting medium with DAPI (Burlingame, Calif.) and viewed using an LSM 510 Zeiss confocal microscope at 60× magnification. Images were processed using the software program Image J.

Fluorescence recovery after photobleaching (FRAP)—COS7 cells transiently expressing Runx2-GFP were grown in media with CSS. One hour prior to the FRAP experiment DHT or vehicle was added to the growth media. Cells were viewed using an LSM 510 Zeiss confocal microscope at 60× magnification. A round nuclear section measuring 3.5 μm in diameter was monitored at a wavelength of 488 nm at 4% intensity for 15 seconds prior to a 5 second-long photobleaching with an 80% laser power. Images were subsequently captured each second for one minute at 488 nm (4% intensity) to assess the fluorescence recovery. All data obtained were normalized to the average starting intensity and data was corrected for loss of background fluorescence. Images were processed using the software program Image J.

Electrophoretic mobility shift assay (EMSA)—³²P-labeled double-stranded oligonucleotide probe containing the OSE2 site (5′-AGCTGCAATCACCAACCACAGCA-3′) was prepared as previously described (78) and 100 fmoles were incubated on ice for 10 minutes with 15 μg of MC3T3-E1 whole cell extract. The incubation buffer contained 20 mM Hepes (pH 7.5), 50 mM KCl, 1 mM MgCl₂, 2 mM EDTA, 2.8% glycerol, and 1 μg salmon sperm DNA. Recombinant purified proteins (2.5 or 7.5 μg) and Runx2 antibodies (4 μg) were included in the binding reaction as indicated. Protein-DNA complexes were resolved by PAGE in 6% native gels containing 5% glycerol and 0.25×TBE buffer.

### REFERENCES

1. Komori T, Yagi H, Nomura S, Yamaguchi A, Sasaki K, Deguchi K, Shimizu Y, Bronson R T, Gao Y H, Inada M, Sato M, Okamoto R, Kitamura Y, Yoshiki S, Kishimoto T 1997 Targeted disruption of Cbfa1 results in a complete lack of bone formation owing to maturational arrest of osteoblasts. Cell 89:755-764
2. Otto F, Thornell A P, Crompton T, Denzel A, Gilmour K C, Rosewell I R, Stamp G W, Beddington R S, Mundlos S, Olsen B R, Selby P B, Owen M J 1997 Cbfa1, a candidate gene for cleidocranial dysplasia syndrome, is essential for osteoblast differentiation and bone development. Cell 89:765-771
3. Enomoto H, Enomoto-Iwamoto M, Iwamoto M, Nomura S, Himeno M, Kitamura Y, Kishimoto T, Komori T 2000 Cbfa1 is a positive regulatory factor in chondrocyte maturation. J Biol Chem 275:8695-8702
4. Yoshida C A, Komori T 2005 Role of Runx proteins in chondrogenesis. Crit Rev Eukaryot Gene Expr 15:243-254
5. Ducy P, Zhang R, Geoffroy V, Ridall A L, Karsenty G 1997 Osf2/Cbfa1: a transcriptional activator of osteoblast differentiation. Cell 89:747-754
6. Liu W, Toyosawa S, Furuichi T, Kanatani N, Yoshida C, Liu Y, Himeno M, Narai S, Yamaguchi A, Komori T 2001 Overexpression of Cbfa1 in osteoblasts inhibits osteoblast maturation and causes osteopenia with multiple fractures. J Cell Biol 155:157-166
7. Geoffroy V, Kneissel M, Fournier B, Boyde A, Matthias P 2002 High bone resorption in adult aging transgenic mice overexpressing cbfa1/runx2 in cells of the osteoblastic lineage. Mol Cell Biol 22:6222-6233
8. Maruyama Z, Yoshida C A, Furuichi T, Amizuka N, Ito M, Fukuyama R, Miyazaki T, Kitaura H, Nakamura K, Fujita T, Kanatani N, Moriishi T, Yamana K, Liu W, Kawaguchi H, Nakamura K, Komori T 2007 Runx2 determines bone maturity and turnover rate in postnatal bone development and is involved in bone loss in estrogen deficiency. Dev Dyn 236:1876-1890
9. Tu Q, Zhang J, Paz J, Wade K, Yang P, Chen J 2008 Haploinsufficiency of Runx2 results in bone formation decrease and different BSP expression pattern changes in two transgenic mouse models. J Cell Physiol 217:40-47
10. Barnes G L, Hebert K E, Kamal M, Javed A, Einhorn T A, Lian J B, Stein G S, Gerstenfeld L C 2004 Fidelity of Runx2 activity in breast cancer cells is required for the generation of metastases-associated osteolytic disease. Cancer Res 64:4506-4513
11. Javed A, Barnes G L, Pratap J, Antkowiak T, Gerstenfeld L C, van Wijnen A J, Stein J L, Lian J B, Stein G S 2005 Impaired intranuclear trafficking of Runx2 (AML3/CBFA1) transcription factors in breast cancer cells inhibits osteolysis in vivo. Proc Natl Acad Sci USA 102:1454-1459
12. Blyth K, Cameron E R, Neil J C 2005 The RUNX genes: gain or loss of function in cancer. Nat Rev Cancer 5:376-387
13. Zaidi S K, Pande S, Pratap J, Gaur T, Grigoriu S, Ali S A, Stein J L, Lian J B, van Wijnen A J, Stein G S 2007 Runx2 deficiency and defective subnuclear targeting bypass senescence to promote immortalization and tumorigenic potential. Proc Natl Acad Sci USA 104:19861-19866
14. Banerjee C, McCabe L R, Choi J Y, Hiebert S W, Stein J L, Stein G S, Lian J B 1997 Runt homology domain proteins in osteoblast differentiation: AML3/CBFA1 is a major component of a bone-specific complex. J Cell Biochem 66:1-8
15. Ducy P, Desbois C, Boyce B, Pinero G, Story B, Dunstan C, Smith E, Bonadio J, Goldstein S, Gundberg C, Bradley A, Karsenty G 1996 Increased bone formation in osteocalcin-deficient mice. Nature 382:448-452
16. Lee N K, Sowa H, Hinoi E, Ferron M, Ahn J D, Confavreux C, Dacquin R, Mee P J, McKee M D, Jung D Y, Zhang Z, Kim J K, Mauvais-Jarvis F, Ducy P, Karsenty G 2007 Endocrine regulation of energy metabolism by the skeleton. Cell 130:456-469
17. Vaes B L, Ducy P, Sijbers A M, Hendriks J M, van Someren E P, de Jong N G, van den Heuvel E R, Olijve W, van Zoelen E J, Dechering K J 2006 Microarray analysis on Runx2-deficient mouse embryos reveals novel Runx2 functions and target genes during intramembraneous and endochondral bone formation. Bone 39:724-738
18. Hecht J, Seitz V, Urban M, Wagner F, Robinson P N, Stiege A, Dieterich C, Kornak U, Wilkening U, Brieske N, Zwingman C, Kidess A, Stricker S, Mundlos S 2007 Detection of novel skeletogenesis target genes by comprehensive analysis of a Runx2(−/−) mouse model. Gene Expr Patterns 7:102-112
19. Barski A, Frenkel B 2004 ChIP Display: novel method for identification of genomic targets of transcription factors. Nucleic Acids Res 32:e104
20. Pregizer S, Barski A, Gersbach C A, Garcia A J, Frenkel B 2007 Identification of novel Runx2 targets in osteoblasts: cell type-specific BMP-dependent regulation of Tram2. J Cell Biochem 102:1458-1471
21. Westendorf J J, Zaidi S K, Cascino J E, Kahler R, van Wijnen A J, Lian J B, Yoshida M, Stein G S, Li X 2002 Runx2 (Cbfa1, AML-3) interacts with histone deacetylase 6 and represses the p21(CIP1/WAF1) promoter. Mol Cell Biol 22:7982-7992
22. Eliseev R A, Dong Y F, Sampson E, Zuscik M J, Schwarz E M, O'Keefe R J, Rosier R H, Drissi M H 2008 Runx2-mediated activation of the Bax gene increases osteosarcoma cell sensitivity to apoptosis. Oncogene 27:3605-3614
23. Schroeder T M, Jensen E D, Westendorf J J 2005 Runx2: a master organizer of gene transcription in developing and maturing osteoblasts. Birth Defects Res C Embryo Today 75:213-225
24. Kundu M, Javed A, Jeon J P, Horner A, Shum L, Eckhaus M, Muenke M, Lian J B, Yang Y, Nuckolls G H, Stein G S, Liu P P 2002 Cbfbeta interacts with Runx2 and has a critical role in bone development. Nat Genet 32:639-644
25. Sierra J, Villagra A, Paredes R, Cruzat F, Gutierrez S, Javed A, Arriagada G, Olate J, Imschenetzky M, Van Wijnen A J, Lian J B, Stein G S, Stein J L, Montecino M 2003 Regulation of the bone-specific osteocalcin gene by p300 requires Runx2/Cbfa1 and the vitamin D3 receptor but not p300 intrinsic histone acetyltransferase activity. Mol Cell Biol 23:3339-3351
26. Thomas T, Voss A K, Chowdhury K, Gruss P 2000 Querkopf, a MYST family histone acetyltransferase, is required for normal cerebral cortex development. Development 127:2537-2548
27. Gasperowicz M, Otto F 2005 Mammalian Groucho homologs: redundancy or specificity? J Cell Biochem 95:670-687
28. Afzal F, Pratap J, Ito K, Ito Y, Stein J L, van Wijnen A J, Stein G S, Lian J B, Javed A 2005 Smad function and intranuclear targeting share a Runx2 motif required for osteogenic lineage induction and BMP2 responsive transcription. J Cell Physiol 204:63-72
29. Alliston T, Choy L, Ducy P, Karsenty G, Derynck R 2001 TGF-beta-induced repression of CBFA1 by Smad3 decreases cbfa1 and osteocalcin expression and inhibits osteoblast differentiation. Embo J 20:2254-2272
30. Stock M, Otto F 2005 Control of RUNX2 isoform expression: the role of promoters and enhancers. J Cell Biochem 95:506-517
31. Sudhakar S, Li Y, Katz M S, Elango N 2001 Translational regulation is a control point in RUNX2/Cbfa1 gene expression. Biochem Biophys Res Commun 289:616-622
32. Bae S C, Lee Y H 2006 Phosphorylation, acetylation and ubiquitination: the molecular basis of RUNX regulation. Gene 366:58-66
33. Zaidi S K, Javed A, Choi J Y, van Wijnen A J, Stein J L, Lian J B, Stein G S 2001 A specific targeting signal directs Runx2/Cbfa1 to subnuclear domains and contributes to transactivation of the osteocalcin gene. J Cell Sci 114:3093-3102
34. Pregizer S, Baniwal S K, Yan X, Borok Z, Frenkel B 2008 Progressive recruitment of Runx2 to genomic targets despite decreasing expression during osteoblast differentiation. J Cell Biochem 105:965-970
35. Zhang Y W, Yasui N, Ito K, Huang G, Fujii M, Hanai J, Nogami H, Ochi T, Miyazono K, Ito Y 2000 A RUNX2/PEBP2alpha A/CBFA1 mutation displaying impaired transactivation and Smad interaction in cleidocranial dysplasia. Proc Natl Acad Sci USA 97:10549-10554
36. Ge C, Xiao G, Jiang D, Franceschi R T 2007 Critical role of the extracellular signal-regulated kinase-MAPK pathway in osteoblast differentiation and skeletal development. J Cell Biol 176:709-718
37. Xiao G, Jiang D, Thomas P, Benson M D, Guan K, Karsenty G, Franceschi R T 2000 MAPK pathways activate and phosphorylate the osteoblast-specific transcription factor, Cbfa1. J Biol Chem 275:4453-4459
38. Qiao M, Shapiro P, Kumar R, Passaniti A 2004 Insulin-like growth factor-1 regulates endogenous RUN-X2 activity in endothelial cells through a phosphatidylinositol 3-kinase/ERK-dependent and Akt-independent signaling pathway. J Biol Chem 279:42709-42718
39. Ruizeveld de Winter J A, Janssen P J, Sleddens H M, Verleun-Mooijman M C, Trapman J, Brinkmann A O, Santerse A B, Schroder F H, van der Kwast T H 1994 Androgen receptor status in localized and locally progressive hormone refractory human prostate cancer. Am J Pathol 144:735-746
40. Sadi M V, Walsh P C, Barrack E R 1991 Immunohistochemical study of androgen receptors in metastatic prostate cancer. Comparison of receptor content and response to hormonal therapy. Cancer 67:3057-3064
41. Chodak G W, Kranc D M, Puy L A, Takeda H, Johnson K, Chang C 1992 Nuclear localization of androgen receptor in heterogeneous samples of normal, hyperplastic and neoplastic human prostate. J Urol 147:798-803
42. Heinlein C A, Chang C 2004 Androgen receptor in prostate cancer. Endocr Rev 25:276-308
43. Dehm S M, Tindall D J 2007 Androgen receptor structural and functional elements: role and regulation in prostate cancer. Mol Endocrinol 21:2855-2863
44. Leder B Z, LeBlanc K M, Schoenfeld D A, Eastell R, Finkelstein J S 2003 Differential effects of androgens and estrogens on bone turnover in normal men. J Clin Endocrinol Metab 88:204-210
45. Vanderschueren D, Vandenput L, Boonen S, Lindberg M K, Bouillon R, Ohlsson C 2004 Androgens and bone. Endocr Rev 25:389-425
46. Kawano H, Sato T, Yamada T, Matsumoto T, Sekine K, Watanabe T, Nakamura T, Fukuda T, Yoshimura K, Yoshizawa T, Aihara K, Yamamoto Y, Nakamichi Y, Metzger D, Chambon P, Nakamura K, Kawaguchi H, Kato S 2003 Suppressive function of androgen receptor in bone resorption. Proc Natl Acad Sci USA 100:9416-9421
47. Syed F, Khosla S 2005 Mechanisms of sex steroid effects on bone. Biochem Biophys Res Commun 328:688-696
48. Orwoll E S, Klein R F 1995 Osteoporosis in men. Endocr Rev 16:87-116
49. Bland R 2000 Steroid hormone receptor expression and action in bone. Clin Sci (Lond) 98:217-240
50. Yeh S, Tsai M Y, Xu Q, Mu X M, Lardy H, Huang K E, Lin H, Yeh S D, Altuwaijri S, Zhou X, Xing L, Boyce B F, Hung M C, Zhang S, Gan L, Chang C 2002 Generation and characterization of androgen receptor knockout (ARKO) mice: an in vivo model for the study of androgen functions in selective tissues. Proc Natl Acad Sci USA 99:13498-13503
51. Khalid O, Baniwal S K, Purcell D J, Leclerc N, Gabet Y, Stallcup M R, Coetzee G A, Frenkel B 2008 Modulation of Runx2 activity by estrogen receptor-alpha: implications for osteoporosis and breast cancer. Endocrinology 149:5984-5995
52. Vaughan T, Pasco J A, Kotowicz M A, Nicholson G C, Morrison N A 2002 Alleles of RUNX2/CBFA1 gene are associated with differences in bone mineral density and risk of fracture. J Bone Miner Res 17:1527-1534
53. Ducy P, Karsenty G 1995 Two distinct osteoblast-specific cis-acting elements control expression of a mouse osteocalcin gene. Mol Cell Biol 15:1858-1869
54. Ning Y M, Robins D M 1999 AML3/CBFalpha1 is required for androgen-specific activation of the enhancer of the mouse sex-limited protein (Sip) gene. J Biol Chem 274:30624-30630
55. Kawate H, Wu Y, Ohnaka K, Takayanagi R 2007 Mutual transactivational repression of Runx2 and the androgen receptor by an impairment of their normal compartmentalization. J Steroid Biochem Mol Biol 105:46-56
56. Harrington K S, Javed A, Drissi H, McNeil S, Lian J B, Stein J L, Van Wijnen A J, Wang Y L, Stein G S 2002 Transcription factors RUNX1/AML1 and RUNX2/Cbfa1 dynamically associate with stationary subnuclear domains. J Cell Sci 115:4167-4176
57. Stein G S, van Wijnen A J, Stein J L, Lian J B, Montecino M, Choi J, Zaidi K, Javed A 2000 Intranuclear trafficking of transcription factors: implications for biological control. J Cell Sci 113 (Pt 14):2527-2533
58. Buchanan G, Craft P S, Yang M, Cheong A, Prescott J, Jia L, Coetzee G A, Tilley W D 2004 PC-3 cells with enhanced androgen receptor signaling: A model for clonal selection in prostate cancer. Prostate 60:352-366
59. Holzbeierlein J, Lal P, LaTulippe E, Smith A, Satagopan J, Zhang L, Ryan C, Smith S, Scher H, Scardino P, Reuter V, Gerald W L 2004 Gene expression analysis of human prostate carcinoma during hormonal therapy identifies androgen-responsive genes and mechanisms of therapy resistance. Am J Pathol 164:217-227
60. Takeuchi M, Kakushi H, Tohkin M 1994 Androgens directly stimulate mineralization and increase androgen receptors in human osteoblast-like osteosarcoma cells. Biochem Biophys Res Commun 204:905-911
61. Wiren K M, Zhang X, Chang C, Keenan E, Orwoll E S1997 Transcriptional up-regulation of the human androgen receptor by androgen in bone cells. Endocrinology 138:2291-2300
62. Backstrom S, Wolf-Watz M, Grundstrom C, Hard T, Grundstrom T, Sauer U H 2002 The RUNX1 Runt domain at 1.25 A resolution: a structural switch and specifically bound chloride ions modulate DNA binding. J Mol Biol 322:259-272
63. Wiren K M, Zhang X W, Toombs A R, Kasparcova V, Gentile M A, Harada S, Jepsen K J 2004 Targeted overexpression of androgen receptor in osteoblasts: unexpected complex bone phenotype in growing animals. Endocrinology 145:3507-3522
64. Javed A, Guo B, Hiebert S, Choi J Y, Green J, Zhao S C, Osborne M A, Stifani S, Stein J L, Lian J B, van Wijnen A J, Stein G S 2000 Groucho/TLE/R-esp proteins associate with the nuclear matrix and repress RUNX (CBF(alpha)/AML/PEBP2(alpha)) dependent activation of tissue-specific gene transcription. J Cell Sci 113 (Pt 12):2221-2231
65. Paredes R, Arriagada G, Cruzat F, Villagra A, Olate J, Zaidi K, van Wijnen A, Lian J B, Stein G S, Stein J L, Montecino M 2004 Bone-specific transcription factor Runx2 interacts with the 1alpha,25-dihydroxyvitamin D3 receptor to up-regulate rat osteocalcin gene expression in osteoblastic cells. Mol Cell Biol 24:8847-8861
66. Cameron E R, Neil J C 2004 The Runx genes: lineage-specific oncogenes and tumor suppressors. Oncogene 23:4308-4314
67. Pratap J, Lian J B, Javed A, Barnes G L, van Wijnen A J, Stein J L, Stein G S 2006 Regulatory roles of Runx2 in metastatic tumor and cancer cell interactions with bone. Cancer Metastasis Rev 25:589-600
68. Riggs B L, Khosla S, Melton L J, 3rd 2002 Sex steroids and the construction and conservation of the adult skeleton. Endocr Rev 23:279-302
69. Enomoto H, Shiojiri S, Iloshi K, Furuichi T, Fukuyama R, Yoshida C A, Kanatani N, Nakamura R, Mizuno A, Zanma A, Yano K, Yasuda H, Higashio K, Takada K, Komori T 2003 Induction of osteoclast differentiation by Runx2 through receptor activator of nuclear factor-kappa B ligand (RANKL) and osteoprotegerin regulation and partial rescue of osteoclastogenesis in Runx2−/− mice by RANKL transgene. J Biol Chem 278:23971-23977
70. Smith E P, Boyd J, Frank G R, Takahashi H, Cohen R H, Specker B, Williams T C, Lubahn D B, Korach K S 1994 Estrogen resistance caused by a mutation in the estrogen-receptor gene in a man. N Engl J Med 331:1056-1061
71. Morishima A, Grumbach M M, Simpson E R, Fisher C, Qin K 1995 Aromatase deficiency in male and female siblings caused by a novel mutation and the physiological role of estrogens. J Clin Endocrinol Metab 80:3689-3698
72. Carani C, Qin K, Simoni M, Faustini-Fustini M, Serpente S, Boyd J, Korach K S, Simpson E R 1997 Effect of testosterone and estradiol in a man with aromatase deficiency. N Engl J Med 337:91-95
73. Falahati-Nini A, Riggs B L, Atkinson E J, O'Fallon W M, Eastell R, Khosla S 2000 Relative contributions of testosterone and estrogen in regulating bone resorption and formation in normal elderly men. J Clin Invest 106:1553-1560
74. Jia L, Shen H C, Wantroba M, Khalid O, Liang G, Wang Q, Gentzschein E, Pinski J K, Stanczyk F Z, Jones P A, Coetzee G A 2006 Locus-wide chromatin remodeling and enhanced androgen receptor-mediated transcription in recurrent prostate tumor cells. Mol Cell Biol 26:7331-7341
75. Zufferey R, Nagy D, Mandel R J, Naldini L, Trono D 1997 Multiply attenuated lentiviral vector achieves efficient gene delivery in vivo. Nat Biotechnol 15:871-875
76. Kim J H, Yang C R, Heo K, Roeder R G, An W, Stallcup M R 2008 CCAR1, a key regulator of mediator complex recruitment to nuclear receptor transcription complexes. Mol Cell 31:510-519
77. Baniwal S K, Chan K Y, Scharf K D, Nover L 2007 Role of heat stress transcription factor HsfA5 as specific repressor of HsfA4. J Biol Chem 282:3605-3613
78. Luppen C A, Smith E, Spevak L, Boskey A L, Frenkel B 2003 Bone morphogenetic protein-2 restores mineralization in glucocorticoid-inhibited MC3T3-E1 osteoblast cultures. J Bone Miner Res 18:1186-1197

### Example III

### The Role of Transcription Factors Runx2 and AR in Prostate Cancer

### Abstract

The androgen receptor (AR) and Runx proteins are transcription factors important in prostate cancer etiology and bone differentiation, respectively. We discovered an important link between them by demonstrating their physical interactions that have important functional consequences. It is believed that the oncogenic actions of the AR may partly be due to its inhibition of the tumor suppressor activities of Runx2, while the tumor suppressor actions of Runx2 may partly be due to its inhibition of the oncogenic activities of the AR. The interplay between the two transcription factors is the main focus of this study.

### Introduction

AR signaling is critical in all phases of PCa, including disease initiation, androgen-dependent tumor growth and the evolution of resistance to androgen ablation therapies. Aberrant AR signaling alone can cause PCa as demonstrated by expressing a mutant AR in the prostate of transgenic mice (1); the mutant AR had higher than normal transcriptional activity in the absence of ligand and had increased sensitivity to coregulators. Mice carrying this mutant, but not a wild-type AR transgene, subsequently developed PCa in 100% of cases. The Sawyers' lab (2) reported that two- to five-fold increases in AR expression were consistently associated with the development of cells that had become supersensitive to androgen resulting in an ablation-resistant phenotype. Furthermore, reactivation of the androgen responsive pathway in ablation-resistant human tumors was also the major finding in another expression array study (3). Even an unbiased network biology approach in which reverse-engineered gene networks were combined with expression profiles identified the AR as among the top genetic mediators and the AR pathway as a highly enriched pathway for metastatic PCa (4).

Runx2, otherwise an osteoblast master transcription factor, has also come to the forefront in the PCa field due to its potential role in PCa bone metastasis and its direct interaction with the AR. Runx2 promotes osteoblast differentiation and bone formation (5-7). Although Runx2 has been implicated in certain cancers (8-10), its precise roles in the etiology of prostate cancer in either primary or metastatic settings are unknown. The significance of Runx2 expression in PCa cells and the outcome of the physical interaction between the AR and Runx2 are also unknown and the main focus of the present study.

### Objectives

One object is to decipher the role that the interaction between the AR and Runx2 plays in various stages of PCa progression. Our data show that the AR and Runx2 inhibit each other, and it is believed that these inhibitory activities play important roles in various stages of tumor progression in vivo. To develop tools for in vivo studies, our immediate objective is to dissect the molecular basis and the transcriptional outcome of the interaction between AR and Runx2. It is believed that the AR and Runx2 interact with each other through specific domains and that these interactions result in the inhibition of their respective DNA binding activities and the transcription of respective target genes.

More specifically, one object is to map the AR and Runx2 interaction surfaces. Another object is to test whether AR counteracts Runx2 interaction with and activation of transfected and endogenous target genes. Yet another object is to test whether Runx2 counteracts AR interaction with and activation of transfected and endogenous target genes.

AR/Runx2 interaction assays and site-directed mutagenesis have been performed. AR/Runx2 reciprocal inhibition on natural promoters and AR/Runx2 occupancies of target genes in living PCa cells will be investigated. Selected AR/Runx2 mutants in functional assays will be analyzed.

### Results

### AR/Runx2 Interaction Assays and Site-Directed Mutagenesis

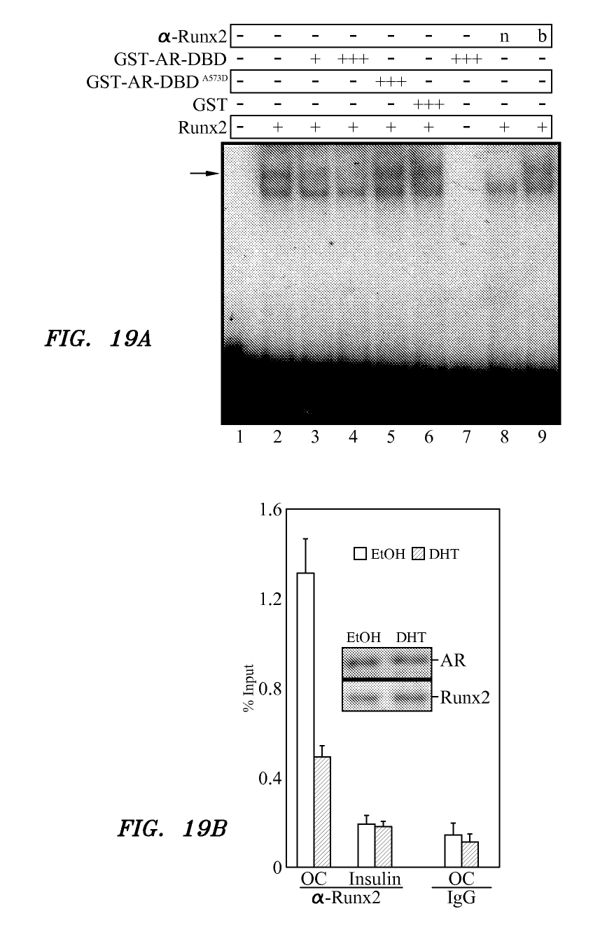

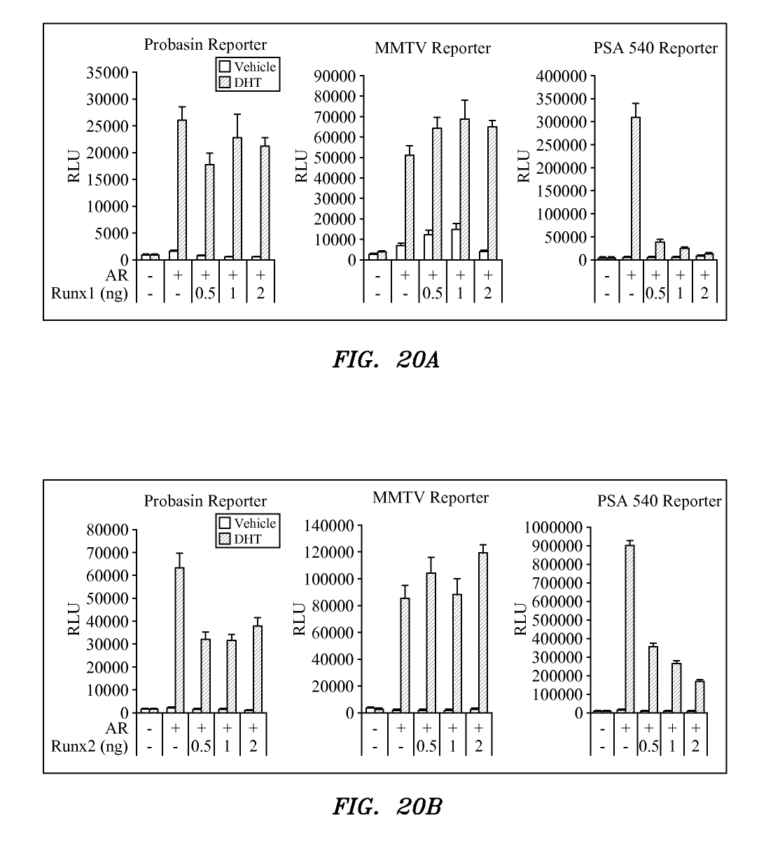

Runx1 and 2 inhibit AR activity in a promoter dependent fashion: We have previously shown that the AR inhibited Runx2 activity. We now assessed the reverse, namely whether Runx proteins are able to inhibit AR activity (FIG. 20). Interestingly this was the case with both Runx proteins in a ligand dependent way. However the inhibition was very much promoter-dependent. Thus, AR transactivation activity was inhibited on the two natural promoters probasin and PSA) by especially Runx2 (more so on PSA promoter), Interestingly, on the viral promoter (MMTV) this inhibition did not occur. Also, although Runx2 inhibited AR activity somewhat on both probasin and PSA promoters, Runx1's inhibition was only apparent on the PSA promoter where the inhibition was the most dramatic. These results indicate that binding sites for other transcription factors on these promoters may play a role in Runx inhibition of the AR. Such qualitative differences may well be important in the etiology of prostate cancer.

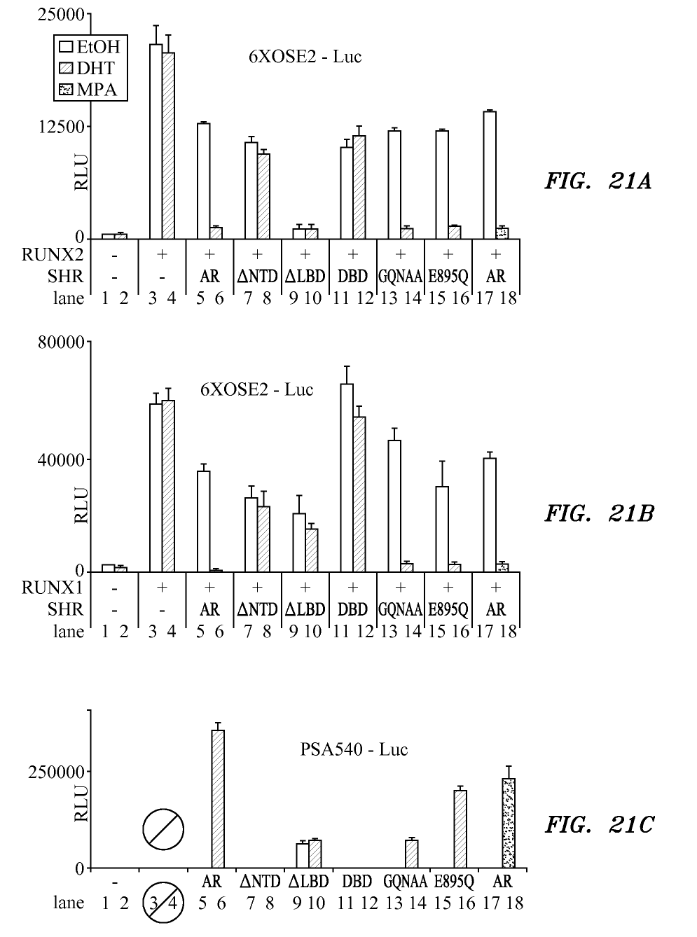

Neither AR transactivation activity nor its N/C interaction is necessary for Runx inhibition: A series of AR mutants were tested for Runx2 (FIG. 21A) and Runx1 (FIG. 21B) inhibition. Although the ΔLBD construct had very little transactivation activity, it inhibited especially Runx2 as well as wtAR. Also since neither ΔNBD nor DBD alone inhibited Runx2 activity, the LBD most likely expose interaction domains in the other parts of the molecule upon ligand binding. The GQNAA and E895Q mutants or MPA-stimulated AR are all known to have no AR N/C interaction activity, and yet were all inhibited Runx activities. The results indicate AR N/C activity is not necessary for Runx inhibition.

Next we checked whether the DHT dependence of AR-mediated transactivation activity and Runx2 inhibition followed similar kinetics (FIG. 22). This was not the case and the ED₅₀ value for DHT activation of the AR (3.19 nM) was an order of magnitude bigger than the ED₅₀ value of DHT-mediated inhibition of Runx2 activity (0.27 nM) (FIG. 22). Taken together with the mutant data recorded in FIG. 21, it seems very unlikely that the Runx inhibition by the AR is mediated via an intermediary protein synthesized under AR and DHT control. Much more likely is a direct interaction between AR and Runx proteins and that this complex inhibits both transcription factors' abilities to mediate transcriptional control on their respective promoters. Therefore, at this stage we decided to assess AR and Runx interaction directly using Co-IP, GST-pull-down and immunofluorescence assays.

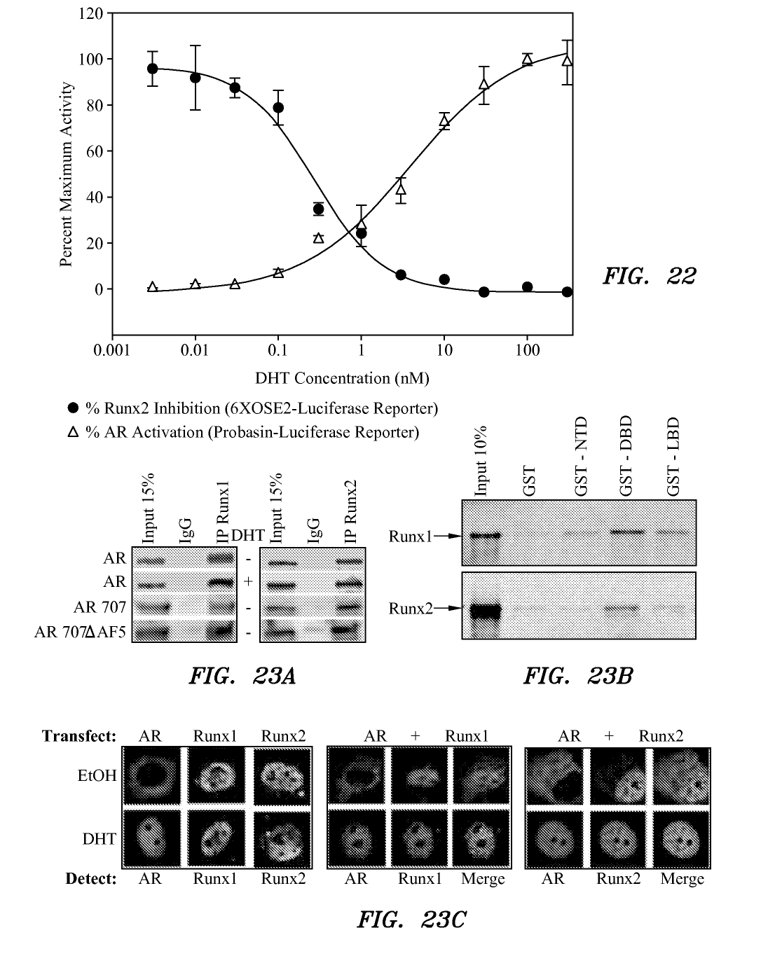

Co-IP analyses indicated that Runx1 & 2 were both able to interact with the AR in a ligand independent way and even after large parts of the LBD (AR707) and NTD (AR707ΔAF5) were deleted (FIG. 23A). In in vitro GST pull-down assays the GST-DBD was the main interacting AR subdomain (FIG. 23B), indicating that the DBD has the main interacting surface with the AR. Co-localization analyses clearly showed that the cytoplasmic AR (in the absence of ligand) is efficiently translocated to the nucleus by the addition of the natural ligand DHT (FIG. 23C). It is in the nucleus that the two transcription factors meet and presumably affect each other's activity. These results were obtained in transfected COS7 cells. We next turned to prostate cancer cells (PC3).

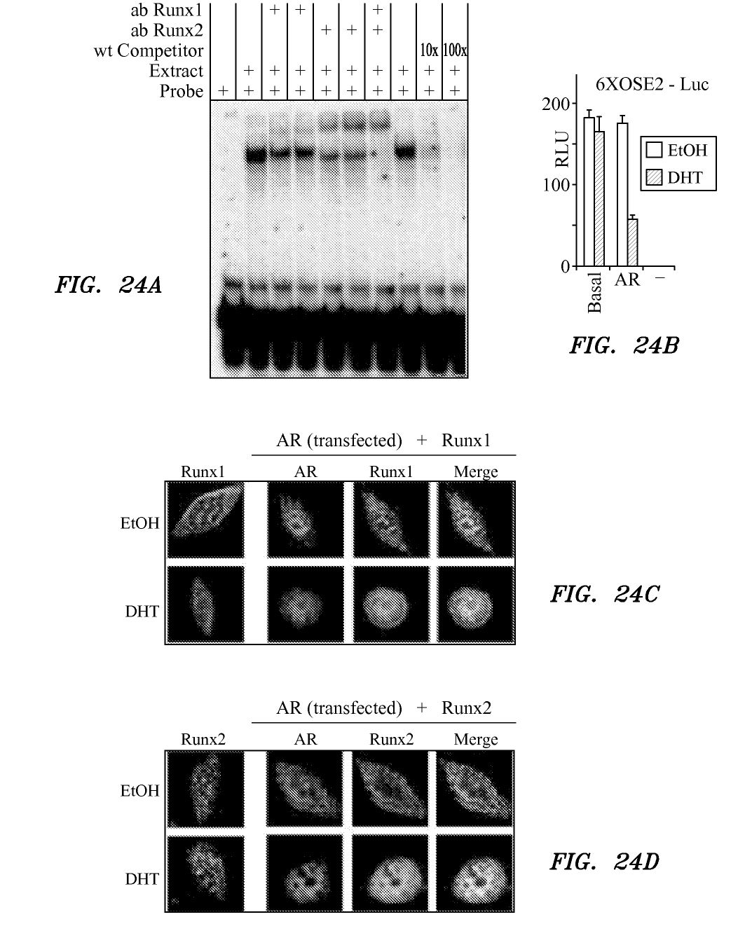

The endogenous Runx proteins in prostate cancer cells (PC3) were active, since they were able to shift a Runx promoter element in electromobility shift assays (EMSA) (FIG. 24A). The shifted bands were further super-shifted by both Runx1 and Runx2 antibodies and were successfully competed with unlabeled probe indicating the specificity of the interactions. The endogenous Runx-mediated activity was also inhibited by transfected AR and DHT treatment (FIG. 24B). Immunofluorescence confocal analyses indicated that the endogenous Runx proteins in the PC3 cells were localized in both the cytoplasm and nucleus (these cells do not contain endogenous AR). Transfected AR and DHT treatment combined were able to translocate both proteins to the nucleus where they presumably influence each other's activities. Therefore, the physical interactions between the two transcription factors have clear in vivo consequences.

In summary, we have established that the real and functional interactions between the two important transcription factors may have consequences in prostate cancer physiology.

### REFERENCES

1+Han, G., Buchanan, G., Ittmann, M., Harris, J. M., Yu, X., Demayo, F. J., Tilley, W. & Greenberg, N. M. Mutation of the androgen receptor causes oncogenic transformation of the prostate. Proc Natl Acad Sci USA 102, 1151-6 (2005).
2. Chen, C. D., Welsbie, D. S., Tran, C., Baek, S. H., Chen, R., Vessella, R., Rosenfeld, M. G. & Sawyers, C. L. Molecular determinants of resistance to antiandrogen therapy. Nat Med 10, 33-39 (2004).
3. Holzbeierlein, J., Lal, P., LaTulippe, E., Smith, A., Satagopan, J., Zhang, L., Ryan, C., Smith, S., Scher, H., Scardino, P., Reuter, V. & Gerald, W. L. Gene Expression Analysis of Human Prostate Carcinoma during Hormonal Therapy Identifies Androgen-Responsive Genes and Mechanisms of Therapy Resistance. Am J Pathol 164, 217-27 (2004).
4. Ergun, A., Lawrence, C. A., Kohanski, M. A., Brennan, T. A. & Collins, J. J. A network biology approach to prostate cancer. Mol Syst Biol 3, 82 (2007).
5. Deng, Z. L., Sharff, K. A., Tang, N., Song, W. X., Luo, J., Luo, X., Chen, J., Bennett, E., Reid, R., Manning, D., Xue, A., Montag, A. G., Luu, H. H., Haydon, R. C. & He, T. C. Regulation of osteogenic differentiation during skeletal development. Front Biosci 13, 2001-21 (2008).
6. Karsenty, G. Minireview: transcriptional control of osteoblast differentiation. Endocrinology 142, 2731-3 (2001).
7. Lian, J. B. & Stein, G. S. Runx2/Cbfa1: a multifunctional regulator of bone formation. Curr Pharm Des 9, 2677-85 (2003).
8. Cicek, M. & Oursler, M. J. Breast cancer bone metastasis and current small therapeutics. Cancer Metastasis Rev 25, 635-44 (2006).
9. Pratap, J., Lian, J. B., Javed, A., Barnes, G. L., van Wijnen, A. J., Stein, J. L. & Stein, G. S. Regulatory roles of Runx2 in metastatic tumor and cancer cell interactions with bone. Cancer Metastasis Rev 25, 589-600 (2006).
10. Shore, P. A role for Runx2 in normal mammary gland and breast cancer bone metastasis. J Cell Biochem 96, 484-9 (2005).
11. Ya-Huei Kuo, Sayyed K Zaidi, Svetlana Gornostaeva, Toshihisa Komori, Gary S. Stein and Lucio H. Runx2 induces acute myeloid leukemia in cooperation with Cbf{beta}-SMMHC in mice. Blood. Published online on Jan. 28, 2009.

### Example IV

### Androgens, but not Estrogens, Inhibit Runx1

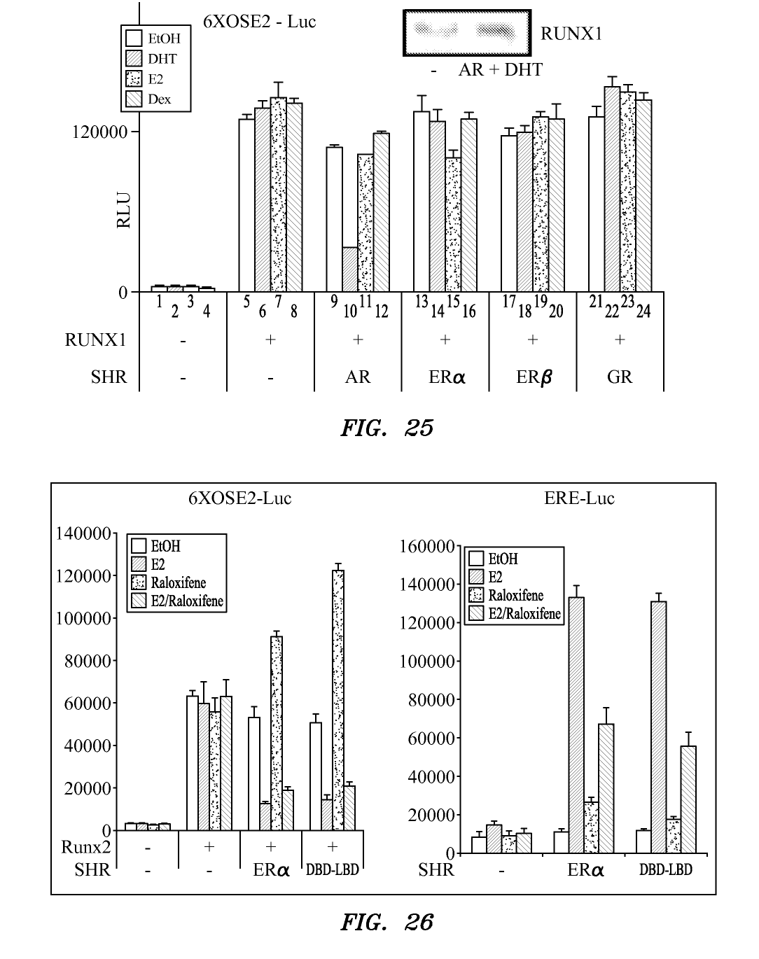

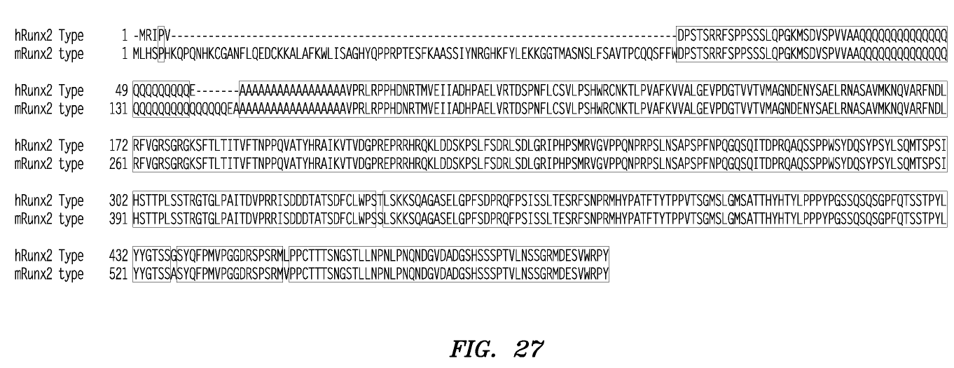

Runx2 and Runx1 have similar (93% homologous) DNA-binding (“Runt-homology”) domains (1), which recognize the same DNA motif (2). Therefore, although Runx2 is the transcription factor most strongly implicated in osteoblastogenesis, it is not surprising that Runx1 was also implicated in this process (3, 4) [as well as in chondrogenesis (5)]. We tested the effects of ERαX, ERβ, AR and GR on Runx1 activity using the same assay employed for Runx2. Runx1 stimulated transcription from 6XOSE2-luc similar to Runx2 (FIG. 25, lane 5 vs. 1). Surprisingly, however, only AR+DHT robustly inhibited Runx1 activity (FIG. 25, lane 10 vs. 5), approaching the inhibition levels previously seen with Runx2 (Khalid et al, 2008). Similar to the inhibition of Runx2, the inhibition of Runx1 by AR was primarily ligand-dependent (FIG. 25, lanes 5, 9 and 10), with the ligand-independent repression ranging between 15% and 60% in multiple independent experiments. The other receptors, including ERβ, GR, and most importantly ERα, whether activated by ligand or not, inhibited Runx1 only minimally or not at all (FIG. 25). The unique ability of AR to inhibit Runx1 (in addition to Runx2) may help explain the extra pro-skeletal protection of androgens beyond what is attributable to aromatization (6-8).

### REFERENCES

1. Westendorf, J. J. & Hiebert, S. W. Mammalian runt-domain proteins and their roles in hematopoiesis, osteogenesis, and leukemia. J Cell Biochem Suppl, 51-8 (1999).
2. Melnikova, I. N., Crute, B. E., Wang, S. & Speck, N. A. Sequence specificity of the core-binding factor. J Virol 67, 2408-11 (1993).
3. Lian, J. B., Balint, E., Javed, A., Drissi, H., Vitti, R., Quinlan, E. J., Zhang, L., Van Wijnen, A. J., Stein, J. L., Speck, N. & Stein, G. S. Runx1/AML1 hematopoietic transcription factor contributes to skeletal development in vivo J Cell Physiol 196, 301-11 (2003).
4. Yamashiro, T., Wang, X. P., Li, Z., Oya, S., Aberg, T., Fukunaga, T., Kamioka, H., Speck, N. A., Takano-Yamamoto, T. & Thesleff, I. Possible roles of Runx1 and Sox9 in incipient intramembraneous ossification. J Bone Miner Res 19, 1671-7 (2004).
5. Wang, Y., Belflower, R. M., Dong, Y. F., Schwarz, E. M., O'Keefe, R. J. & Drissi, H. Runx1/AML1/Cbfa2 mediates onset of mesenchymal cell differentiation toward chondrogenesis. J Bone Miner Res 20, 1624-36 (2005).
6. Leder, B. Z., LeBlanc, K. M., Schoenfeld, D. A., Eastell, R. & Finkelstein, J. S. Differential effects of androgens and estrogens on bone turnover in normal men. J Clin Endocrinol Metab 88, 204-10 (2003).
7. Kawano, H., Sato, T., Yamada, T., Matsumoto, T., Sekine, K., Watanabe, T., Nakamura, T., Fukuda, T., Yoshimura, K., Yoshizawa, T., Aihara, K., Yamamoto, Y., Nakamichi, Y., Metzger, D., Chambon, P., Nakamura, K., Kawaguchi, H. & Kato, S. Suppressive function of androgen receptor in bone resorption. Proc Natl Acad Sci USA 100, 9416-21 (2003).
8. Vanderschueren, D., Vandenput, L., Boonen, S., Lindberg, M. K., Bouillon, R. & Ohlsson, C. Androgens and bone. Endocr Rev 25, 389-425 (2004).

### Example V

### Raloxifen-Bound ERαSlightly Stimulates Runx2 Activity in COS7 Cells

COS7 cells were transiently transfected with the indicated expression vectors along with a reporter for either Runx2 (6XOSE2-luciferase) or the classical transcriptional activation activity of ER (ERE-luciferase). Cells were treated for 24 hrs with either vehicle (EtOH), 10 nm E2, 100 nM Raloxifene, or the indicated combinations, followed by cell lysis and luciferase assay.

Note that Raloxifene has only a small effect on Runx2 activity in COS7 cells. Importantly, however, this small effect is stimulatory, the opposite of the small inhibitory effect of Tamoxifene in the same cells (see Khalid et al., 2008, FIG. 6B). Possibly, Runx2 activity in breast cancer cells will also be differentially regulated by these two SERMs.

All publications cited herein are incorporated by reference in their entirety.

What is claimed is:

1. A method of identifying a candidate compound for reducing the activity of a Runx2 (Runt-related transcription factor 2) protein in a cell, comprising:
providing a system containing a Runx2 protein and an ERα (estrogen receptor α) protein or an AR (androgen receptor) protein;
contacting the system with a test compound; and
determining the inhibition of the activity of the Runx2 protein by the ERα protein or the AR protein in the system, wherein the inhibition of the activity of the Runx2 protein by the ERα protein or the AR protein in the system, if increased compared to that before the contacting step, indicates that the test compound is a candidate for reducing the activity of the Runx2 protein in a cell.

2. The method of claim 1, wherein the system is a cell.

3. The method of claim 2, wherein the cell is osteoblastic.

4. The method of claim 1, wherein the test compound is a SERM (selective estrogen receptor modulator) or SARM (selective androgen receptor modulator).

5. A method of reducing the activity of a Runx2 protein in a cell, comprising:
providing a system containing a Runx2 protein and an ERα protein or an AR protein; and
contacting the system with a compound identified according to the method of claim 1.

6. A method of identifying a candidate compound for modulating the activity of a Runx2 protein in a cancer cell, comprising:
providing a cancer cell containing a Runx2 protein and an ERα protein or an AR protein;
contacting the cell with a test compound; and
determining the inhibition of the activity of the Runx2 protein by the ERα protein or the AR protein in the cell, wherein the inhibition of the activity of the Runx2 protein by the ERα protein or the AR protein in the cell, if different from that before the contacting step, indicates that the test compound is a candidate for modulating the activity of the Runx2 protein in the cancer cell.

7. The method of claim 6, wherein the cancer is breast cancer or prostate cancer.

8. The method of claim 6, wherein the cancer cell is an early stage cancer cell.

9. The method of claim 6, wherein the cancer cell is a late stage cancer cell.

10. The method of claim 6, wherein the test compound is a SERM or SARM.

11. A method of identifying a candidate compound for reducing the activity of a Runx1 (Runt-related transcription factor 1) protein in a cell, comprising:
providing a system containing a Runx1 protein and an AR protein;
contacting the system with a test compound; and
determining the inhibition of the activity of the Runx1 protein by the AR protein in the system, wherein the inhibition of the activity of the Runx1 protein by the AR protein in the system, if increased compared to that before the contacting step, indicates that the test compound is a candidate for reducing the activity of the Runx1 protein in a cell.

12. The method of claim 11, wherein the system is a cell.

13. The method of claim 12, wherein the cell is osteoblastic.

14. The method of claim 11, wherein the test compound is a SARM.

15. A method of reducing the activity of a Runx1 protein in a cell, comprising:
providing a system containing a Runx1 protein and an AR protein; and
contacting the system with a compound identified according to the method of claim 11.

16. A method of identifying a candidate compound for modulating the activity of a Runx1 protein in a cancer cell, comprising:
providing a cancer cell containing a Runx1 protein and an AR protein;
contacting the cell with a test compound; and
determining the inhibition of the activity of the Runx1 protein by the AR protein in the cell, wherein the inhibition of the activity of the Runx1 protein by the AR protein in the cell, if different from that before the contacting step, indicates that the test compound is a candidate for modulating the activity of the Runx1 protein in the cancer cell.

17. The method of claim 16, wherein the cancer is leukemia.

18. The method of claim 16, wherein the cancer cell is an early stage cancer cell.

19. The method of claim 16, wherein the cancer cell is a late stage cancer cell.

20. The method of claim 16, wherein the test compound is a SARM.

21. A method of modulating the activity of a Runx1 protein in a cancer cell, comprising:
providing a cancer cell containing a Runx1 protein and an AR protein; and
contacting the cell with a compound identified according to the method of claim 16.


SEQUENCE LISTING (17 sequences)

SEQ ID NO: 1 - DNA, 19 bp, Artificial Sequence
  Synthetic oligonucleotide
cggccctgagtctgacaaa

SEQ ID NO: 2 - DNA, 22 bp, Artificial Sequence
  Synthetic oligonucleotide
gccggagtctgttcactacctt

SEQ ID NO: 3 - DNA, 20 bp, Artificial Sequence
  Synthetic oligonucleotide
ccagggatttcagtcgatgt

SEQ ID NO: 4 - DNA, 20 bp, Artificial Sequence
  Synthetic oligonucleotide
aatctcacgcaggcagttct

SEQ ID NO: 5 - DNA, 21 bp, Artificial Sequence
  Synthetic oligonucleotide
cgctctgttcctgtaccttcg

SEQ ID NO: 6 - DNA, 17 bp, Artificial Sequence
  Synthetic oligonucleotide
aggcttgtttcatcctg

SEQ ID NO: 7 - DNA, 20 bp, Artificial Sequence
  Synthetic oligonucleotide
tacagggccccttccttact

SEQ ID NO: 8 - DNA, 20 bp, Artificial Sequence
  Synthetic oligonucleotide
ctgacgtgggttacctctgg

SEQ ID NO: 9 - DNA, 20 bp, Artificial Sequence
  Synthetic oligonucleotide
cgccgcaagtttctggagac

SEQ ID NO: 10 - DNA, 20 bp, Artificial Sequence
  Synthetic oligonucleotide
cttgccagccttgtttaggc

SEQ ID NO: 11 - DNA, 20 bp, Artificial Sequence
  Synthetic oligonucleotide
ggcagcgaggtagtgaagag

SEQ ID NO: 12 - DNA, 20 bp, Artificial Sequence
  Synthetic oligonucleotide
ctggagaggagcagaactgg

SEQ ID NO: 13 - DNA, 22 bp, Artificial Sequence
  Synthetic oligonucleotide
gtcatgggtgtgaaccatgaga

SEQ ID NO: 14 - DNA, 23 bp, Artificial Sequence
  Synthetic oligonucleotide
ggtcatgagtccttccacgatac

SEQ ID NO: 15 - DNA, 23 bp, Artificial Sequence
  Synthetic oligonucleotide
agctgcaatcaccaaccacagca

SEQ ID NO: 16 - protein, 507 aa, Homo sapiens
MRIPVDPSTSRRFSPPSSSLQPGKMSDVSPVVAAQQQQQQQQQQQQQQQQQQQQQQQEAAAAAAAAAAAAAAAAAVPRLRPPHDNRTMVEIIADHPAELVRTDSPNFLCSVLPSHWRCNKTLPVAFKVVALGEVPDGTVVTVMAGNDENYSAELRNASAVMKNQVARFNDLRFVGRSGRGKSFTLTITVFTNPPQVATYHRAIKVTVDGPREPRRHRQKLDDSKPSLFSDRLSDLGRIPHPSMRVGVPPQNPRPSLNSAPSPFNPQGQSQITDPRQAQSSPPWSYDQSYPSYLSQMTSPSIHSTTPLSSTRGTGLPAITDVPRRISDDDTATSDFCLWPSTLSKKSQAGASELGPFSDPRQFPSISSLTESRFSNPRMHYPATFTYTPPVTSGMSLGMSATTHYHTYLPPPYPGSSQSQSGPFQTSSTPYLYYGTSSGSYQFPMVPGGDRSPSRMLPPCTTTSNGSTLLNPNLPNQNDGVDADGSHSSSPTVLNSSGRMDESVWRPY

SEQ ID NO: 17 - protein, 596 aa, Mus musculus
MLHSPHKQPQNHKCGANFLQEDCKKALAFKWLISAGHYQPPRPTESFKAASSIYNRGHKFYLEKKGGTMASNSLFSAVTPCQQSFFWDPSTSRRFSPPSSSLQPGKMSDVSPVVAAQQQQQQQQQQQQQQQQQQQQQQQQQQQQQEAAAAAAAAAAAAAAAAAAVPRLRPPHDNRTMVEIIADHPAELVRTDSPNFLCSVLPSHWRCNKTLPVAFKVVALGEVPDGTVVTVMAGNDENYSAELRNASAVMKNQVARFNDLRFVGRSGRGKSFTLTITVFTNPPQVATYHRAIKVTVDGPREPRRHRQKLDDSKPSLFSDRLSDLGRIPHPSMRVGVPPQNPRPSLNSAPSPFNPQGQSQITDPRQAQSSPPWSYDQSYPSYLSQMTSPSIHSTTPLSSTRGTGLPAITDVPRRISDDDTATSDFCLWPSSLSKKSQAGASELGPFSDPRQFPSISSLTESRFSNPRMHYPATFTYTPPVTSGMSLGMSATTHYHTYLPPPYPGSSQSQSGPFQTSSTPYLYYGTSSASYQFPMVPGGDRSPSRMVPPCTTTSNGSTLLNPNLPNQNDGVDADGSHSSSPTVLNSSGRMDESVWRPY